SEQUENCE LISTING (30 sequences)

SEQ ID NO: 1 - protein, 15 aa, Artificial Sequence
  linker connecting VL and VH (Kite/iCell anti-CLL1 binders)
GGGGSGGGGSGGGGS

SEQ ID NO: 2 - protein, 22 aa, Artificial Sequence
  P2A element
GSGATNFSLLKQAGDVEENPGP

SEQ ID NO: 3 - protein, 21 aa, Artificial Sequence
  CD8alpha signal peptide
MALPVTALLLPLALLLHAARP

SEQ ID NO: 4 - protein, 45 aa, Artificial Sequence
  CD8alpha hinge
TTTPAPRPPTPAPTIASQPLSLRPEACRPAAGGAVHTRGLDFACD

SEQ ID NO: 5 - protein, 24 aa, Artificial Sequence
  CD8alpha trans-membrane
IYIWAPLAGTCGVLLLSLVITLYC

SEQ ID NO: 6 - protein, 34 aa, Artificial Sequence
  LTalpha signal peptide
MTPPERLFLPRVCGTTLHLLLLGLLLVLLPGAQG

SEQ ID NO: 7 - protein, 205 aa, Artificial Sequence
  LTalpha
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 8 - protein, 144 aa, Artificial Sequence
  LTalpha mutant 1
KPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 9 - protein, 171 aa, Artificial Sequence
  LTalpha mutant 2
LPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 10 - protein, 244 aa, Artificial Sequence
  LTbeta
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 11 - protein, 196 aa, Artificial Sequence
  LTbeta mutant 1
QDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 12 - protein, 158 aa, Artificial Sequence
  LTbeta mutant 2
LPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 13 - protein, 468 aa, Artificial Sequence
  Engineered mutant LTalpha/beta 1
KPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFALGGSGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGGGSGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 14 - protein, 607 aa, Artificial Sequence
  Engineered mutant LTalpha/beta 2
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFALGGGGSQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGGGGGSQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 15 - protein, 621 aa, Artificial Sequence
  Engineered mutant LTalpha/beta 3
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGGGGGSQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGGGGGSLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 16 - protein, 621 aa, Artificial Sequence
  Engineered mutant LTalpha/beta 4
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGGGGGSLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFALGGGGSQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 17 - protein, 240 aa, Artificial Sequence
  LIGHT
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 18 - protein, 40 aa, Artificial Sequence
  CD28 hinge
KIEVMYPPPYLDNEKSNGTIIHVKGKHLCPSPLFPGPSKP

SEQ ID NO: 19 - protein, 27 aa, Artificial Sequence
  CD28 trans-membrane
FWVLVVVGGVLACYSLLVTVAFIIFWV

SEQ ID NO: 20 - protein, 679 aa, Artificial Sequence
  Membrane anchored engineered mutant LTalpha/beta
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFALGGGGSQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGGGGGSQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGGGGGSKIEVMYPPPYLDNEKSNGTIIHVKGKHLCPSPLFPGPSKPFWVLVVVGGVLACYSLLVTVAFIIFWV

SEQ ID NO: 21 - protein, 239 aa, Artificial Sequence
  CD40 antibody (G28.5)
DIQLQQSGPGLVKPSQSLSLTCSVTGYSITTNYNWNWIRQFPGNKLEWMGYIRYDGTSEYTPSLKNRVSITRDTSMNQFFLRLTSVTPEDTATYYCARLDYWGQGTLVTVSSGGGGSGGGGSGGGGSDIVMTQNPLSLPVSLGDEASISCRSSQSLENSNGNTFLNWFFQKPGQSPQLLIYRVSNRFSGVPDRFSGSGSGTDFTLKISRVEAEDLGVYFCLQVTHVPYTFGGGTTLEIK

SEQ ID NO: 22 - protein, 245 aa, Artificial Sequence
  CLL1 antibody (Kite)
DIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRGGGGSGGGGSGGGGSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSS

SEQ ID NO: 23 - protein, 248 aa, Artificial Sequence
  CLL1 antibody (iCell)
DIQMTQSPSSLSASVGDRVTITCRASQSVSTSSYNYMHWYQQKPGKPPKLLIKYASNLESGVPSRFSGSGSGTDFTLTISSLQPEDFATYYCQHSWEIPLTFGQGTKVEIKGGGGSGGGGSGGGGSEVQLVQSGAEVKKPGASVKVSCKASGYSFTDYYMHWVRQAPGQGLEWIGRINPYNGAAFYSQNFKDRVTLTVDTSTSTAYLELSSLRSEDTAVYYCAIERGADLEGYAMDYWGQGTLVTVSS

SEQ ID NO: 24 - protein, 261 aa, Artificial Sequence
  CD40 ligand
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 25 - protein, 124 aa, Artificial Sequence
  anti-GPC2 sdAb
EVQLVESGGGLVQPGGSLRLSCAISEFTYKNTCVGWFRQAPGKGREGVAAIDSDGNTNYVDSVKGRFTISQDNSKNTVYLQMNSLRAEDTAMYYCAAGAYCGRLLLWIGNYAYWGQGTLVTVSS

SEQ ID NO: 26 - protein, 42 aa, Artificial Sequence
  4-1BB co-stimulatroy domain
KRGRKKLLYIFKQPFMRPVQTTQEEDGCSCRFPEEEEGGCEL

SEQ ID NO: 27 - protein, 112 aa, Artificial Sequence
  CD3z
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 5 aa, Artificial Sequence
  Peptide linker
  REPEAT (1)..(5): GSGGS can be repeated n times, where n is an integer of at least one
GSGGS

SEQ ID NO: 29 - protein, 4 aa, Artificial Sequence
  Peptide linker
  REPEAT (1)..(4): GGGS can be repeated n times, where n is an integer of at least one
GGGS

SEQ ID NO: 30 - protein, 5 aa, Artificial Sequence
  Peptide linker
  REPEAT (1)..(5): GGGGS can be repeated n times, where n is an integer of at least one
GGGGS

(19) United States
(12) Patent Application Publication
(10) Pub. No.: US 2024/0041924 A1
(43) Pub. Date: Feb. 8, 2024

(54) T CELL AND ANTIGEN-PRESENTING CELL ENGAGERS AND USES THEREOF

(71) Applicant: Nanjing Legend Biotech Co., Ltd., Nanjing (CN)

(72) Inventors: Fengyuan TANG, Nanjing (CN); Wang Zhang, Nanjing (CN); Yuanyuan Peng, Nanjing (CN); Zhongsheng HU, Nanjing (CN); Shu Wu, Nanjing (CN)

(21) Appl. No.: 18/026,297

(22) PCT Filed: Sep. 29, 2021
(86) PCT No.: PCT/CN2021/121535

§ 371 Date: Mar. 14, 2023

(30) Foreign Application Priority Data
Sep. 29, 2020 (WO) PCT/CN2020/118988

(51) Int. Cl. A61K 35/17; C12N 5/0783; C07K 16/28; C07K 16/24; A61K 39/00; A61P 35/00

(52) U.S. Cl. CPC A61K 35/17; C12N 5/0636; C07K 16/2833; C07K 16/242; C07K 16/2878; C07K 16/2851; A61K 39/4611; A61K 39/4631; A61K 39/464417; A61P 35/00; A61K 2239/13

(57) ABSTRACT

A polypeptide comprising a chimeric antigen receptor (CAR) comprising (i) an extracellular domain capable of binding to a first antigen, (ii) a transmembrane domain, and (iii) an intracellular domain; and a domain capable of binding to a second antigen expressed on the surface of a cell that can interact with a T cell, wherein the CAR and the domain are fused by a peptide linker.

### CROSS REFERENCE TO RELATED APPLICATIONS

This application claims benefit of priority of the International Application No. PCT/CN2020/118988 filed on Sep. 29, 2020, the content of which is incorporated herein by reference in its entirety.

### SEQUENCE LISTING

This application incorporates by reference a Sequence Listing submitted with this application in a text format, entitled “L2-W20230WO_SEQ_LISTING,” created on Sep. 19, 2021 having a size of 54,098 bytes.

### 1. FIELD

Provided herein, in some embodiments, are polypeptides comprising a chimeric antigen receptor fused to a domain capable of binding to a cell surface antigen, e.g., on an antigen-presenting cell (APC), and polynucleotides encoding same. Also provided herein, in certain embodiments, is a composition comprising engineered immune cells expressing a chimeric antigen receptor as well as an additional domain capable of binding to a cell surface antigen of an antigen presenting cell, and uses thereof for treating a disease or disorder, such as cancer.

### 2. BACKGROUND

Adoptive transfer of T cells represents an emerging innovative therapeutic strategy against cancer. For instance, T cells engineered with chimeric antigen receptor (CAR) induce potent clinical response in patients with blood cancers, demonstrating promising superior prognosis comparing with conventional therapies. Nonetheless, the translation of CAR-T cell therapy from liquid tumor to solid tumor is clinically challenging, most probably due to the tumor microenvironment.

Optimal T cell proliferation is driven by antigen presenting cell (APC)-induced CD3ζ and CD28 signals. As such, CAR-T or TCR-T cells deploy these two components to rapidly expand and activate downstream events in response to tumor associate antigen (TAA). Unfortunately, the proliferative capacity of genetically engineered T cells is generally limited, known as exhaustion, due to lack of full spectrum of APC co-stimulations as well as APC derived cytokines. Consequently, the efficacy of CAR-T or TCR-T cells against tumors is greatly impaired due to lack of activated APC in the tumor microenvironment. Several strategies have been designed to overcome these obstacles, none of which however has achieved clinically and practically desirable outcomes.

Therefore, there is still a need in the art for improved constructs or engineered T cells, e.g., CAR-T cells, for treating a disease or disorder such as solid tumor cancer.

### 3. SUMMARY

In one aspect, provided herein is a polypeptide comprising (a) a chimeric antigen receptor (CAR) comprising (i) an extracellular domain capable of binding to a first antigen, (ii) a transmembrane domain, and (iii) an intracellular domain; and (b) a domain capable of binding to a second antigen expressed on the surface of a cell that can interact with a T cell, wherein the CAR and the domain are fused by a peptide linker.

In some embodiments, the cell that can interact with the T cell is capable of presenting the first antigen to the T cell and/or inducing a response from the T cell upon interaction. In some embodiments, the cell that can interact with the T cell is an antigen-presenting cell (APC). In some embodiments, the cell that can interact with the T cell expresses a MHC molecule. In some embodiments, the MHC molecule is a MHC class I molecule. In other embodiments, the MHC molecule is a MHC class II molecule. In some embodiments, the cell that can interact with the T cell is selected from a group consisting of macrophage, dendritic cell, B lymphocyte (B cell), mast cell, basophil, eosinophil, group 3 innate lymphoid cell (ILC3), monocyte, neutrophil, natural killer cell, fibroblastic reticular cell, endothelial cell, pericyte, epithelial cell, fibroblast and artificial APC cell (aAPC). In some embodiments, the cell that can interact with the T cell is an APC cell selected from a group consisting of macrophage, dendritic cell, and B lymphocyte (B cell).

In some embodiments, the first antigen is selected from tumor associated antigen. In some embodiments, the first antigen is selected from a group consisting of 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, BCMA, b-Catenin, bcr-abl, bcr-abl p190 (e1a2), bcr-abl p210 (b2a2), bcr-abl p210 (b3a2), BING-4, CA-125, CAG-3, CAIX, CAMEL, Caspase-8, CD171, CD19, CD20, CD22, CD23, CD24, CD30, CD33, CD38, CD44v7/8, CD70, CD123, CD133, CDC27, CDK-4, CEA, CLCA2, CLL-1, CTAGIB, Cyp-B, DAM-10, DAM-6, DEK-CAN, DLL3, EGFR, EGFRvIII, EGP-2, EGP-40, ELF2, Ep-CAM, EphA2, EphA3, erb-B2, erb-B3, erb-B4, ES-ESO-1a, ETV6/AML, FAP, FBP, fetal acetylcholine receptor, FGF-5, FN, FR-α, G250, GAGE-1, GAGE-2, GAGE-3, GAGE-4, GAGE-5, GAGE-6, GAGE-7B, GAGE-8, GD2, GD3, GnT-V, Gp100, gp75, GPC3, GPC-2, GUCY2C, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, L1-CAM, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, and K-light chain. In some embodiments, the second antigen is a receptor or ligand expressed on the cell that can interact with the T cell. In some embodiments, the second antigen is selected from a group consisting of CD40, CLL1, FLT3, FLT3L, 4-1BB, 4-1BBL, GITR, GITRL, CD27, CD70, OX40, OX40L, PD-1, PD-L1, PD-L2, Galectin-9, B7-H3, B7-H4, ICAM1, ICOS, ICOSL, CD30, CD30L, TIM1, TIM3, TIM4, SEMA4A, CD155, TIGIT, CD160, CD28, CD80, CD86, CTLA4, LAG3, LFA-1, LTβR, and HVEM.

In some embodiments, the second antigen is LTβR. In some embodiments, the domain capable of binding to the second antigen comprises a LTα or variant thereof. In some embodiments, the LTα or variant thereof comprises an amino acid sequence of SEQ ID NO: 7, SEQ ID NO: 8, or SEQ ID NO: 9. In other embodiments, the domain capable of binding to the second antigen comprises a LTβ or variant thereof. In some embodiments, the LTβ or variant thereof comprises an amino acid sequence of SEQ ID NO: 10, SEQ ID NO: 11, or SEQ ID NO: 12. In yet other embodiments, the domain capable of binding to the second antigen comprises a LTα or variant thereof and a LTβ or variant thereof. In some embodiments, the LTα or variant thereof comprises an amino acid sequence of SEQ ID NO: 7, SEQ ID NO: 8, or SEQ ID NO: 9. In some embodiments, the LTβ or variant thereof comprises an amino acid sequence of SEQ ID NO: 10, SEQ ID NO: 11, or SEQ ID NO: 12. In some embodiments, the domain comprises an amino acid sequence of SEQ ID NO: 13, SEQ ID NO: 14, SEQ ID NO: 15, SEQ ID NO: 16, or SEQ ID NO: 20.

In some embodiments, the second antigen is HVEM. In some embodiments, the domain capable of binding to the second antigen comprises a LIGHT (TNFSF14) or variant thereof. In some embodiments, the LIGHT or variant thereof comprises an amino acid sequence of SEQ ID NO: 17.

In other embodiments, the second antigen is CD40. In some embodiments, the domain capable of binding to the second antigen comprises an antibody or fragment thereof that binds CD40. In some embodiments, the antibody or fragment thereof that binds CD40 comprises an amino acid sequence of SEQ ID NO: 21. In some embodiments, the domain capable of binding to the second antigen comprises an amino acid sequence of SEQ ID NO: 24.

In some embodiments, the second antigen is CLL1. In some embodiments, the domain capable of binding to the second antigen comprises an antibody or fragment thereof that binds CLL1. In some embodiments, the antibody or fragment thereof that binds CLL1 comprises an amino acid sequence of SEQ ID NO: 22 or SEQ ID NO: 23.

In some embodiments, the peptide linker is a cleavable peptide linker. In some embodiments, the peptide linker is a 2A self-cleaving peptide. In some embodiments, the 2A self-cleaving peptide is selected from a group consisting of F2A, E2A, P2A, T2A, or variants thereof. In some embodiments, the 2A self-cleaving peptide is a P2A peptide comprising an amino acid sequence of SEQ ID NO: 2.

In some embodiments, the CAR is at the N-terminus of the domain capable of binding to the second antigen. In other embodiments, the CAR is at the C-terminus of the domain capable of binding to the second antigen.

In another aspect, provided herein is a polynucleotide comprising a nucleic acid sequence encoding the polypeptide provided herein.

In another aspect, provided herein is a polynucleotide comprising a first region encoding a CAR comprising (i) an extracellular domain capable of binding to a first antigen, (ii) a transmembrane domain, and (iii) an intracellular domain; and a second region encoding a domain capable of binding to a second antigen expressed on the surface of a cell that can interact with a T cell, wherein the cell that can interact with the T cell is capable of presenting the first antigen to the T cell and/or inducing a response from the T cell upon interaction.

In another aspect, provided herein is a vector comprising a polynucleotide comprising a nucleic acid sequence encoding the polypeptide provided herein.

In yet another aspect, provided herein is a method for making a CAR-T cell, comprising introducing the polynucleotide or the vector provided herein into a T cell.

In yet another aspect, provided herein is a method of making a CAR-T cell comprising introducing into a T cell a polynucleotide comprising a first region encoding a CAR comprising (i) an extracellular domain capable of binding to a first antigen, (ii) a transmembrane domain, and (iii) an intracellular domain; and a second region encoding a domain capable of binding to a second antigen expressed on the surface of a cell that can interact with a T cell, wherein the cell that can interact with the T cell is capable of presenting the first antigen to the T cell and/or inducing a response from the T cell upon interaction.

In yet another aspect, provided herein is a method of making a CAR-T cell comprising introducing into a T cell a first polynucleotide encoding a CAR comprising (i) an extracellular domain capable of binding to a first antigen, (ii) a transmembrane domain, and (iii) an intracellular domain; and a second polynucleotide encoding a domain capable of binding to a second antigen expressed on the surface of a cell that can interact with a T cell, wherein the cell that can interact with the T cell is capable of presenting the first antigen to the T cell and/or inducing a response from the T cell upon interaction.

In yet another aspect, provided herein is a CAR-T cell produced according to the method provided herein.

In yet another aspect, provided herein is a CAR-T cell comprising the polypeptide, the polynucleotide or the vector provided herein.

In yet another aspect, provided herein is a CAR-T cell expressing (a) a CAR comprising (i) an extracellular domain capable of binding to a first antigen, (ii) a transmembrane domain, and (iii) an intracellular domain; and (b) a domain capable of binding to a second antigen expressed on the surface of a cell that can interact with a T cell, wherein the cell that can interact with the T cell is capable of presenting the first antigen to the T cell and/or inducing a response from the T cell upon interaction.

In some embodiments, the cell that can interact with the T cell is an antigen-presenting cell (APC). In some embodiments, the cell that can interact with the T cell expresses a MHC molecule. In some embodiments, the MHC molecule is a MHC class I molecule. In other embodiments, the MHC molecule is a MHC class II molecule. In some embodiments, the cell that can interact with the T cell is selected from a group consisting of macrophage, dendritic cell, B lymphocyte (B cell), mast cell, basophil, eosinophil, group 3 innate lymphoid cell (ILC3), monocyte, neutrophil, natural killer cell, fibroblastic reticular cell, endothelial cell, pericyte, epithelial cell, fibroblast and artificial APC cell (aAPC). In some embodiments, the cell that can interact with the T cell is an APC cell selected from a group consisting of macrophage, dendritic cell, and B lymphocyte (B cell).

In some embodiments, the first antigen is selected from tumor associated antigen. In some embodiments, the first antigen is selected from a group consisting of 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, BCMA, b-Catenin, bcr-abl, bcr-abl p190 (e1a2), bcr-abl p210 (b2a2), bcr-abl p210 (b3a2), BING-4, CA-125, CAG-3, CAIX, CAMEL, Caspase-8, CD171, CD19, CD20, CD22, CD23, CD24, CD30, CD33, CD38, CD44v7/8, CD70, CD123, CD133, CDC27, CDK-4, CEA, CLCA2, CLL-1, CTAGIB, Cyp-B, DAM-10, DAM-6, DEK-CAN, DLL3, EGFR, EGFRvIII, EGP-2, EGP-40, ELF2, Ep-CAM, EphA2, EphA3, erb-B2, erb-B3, erb-B4, ES-ESO-1a, ETV6/AML, FAP, FBP, fetal acetylcholine receptor, FGF-5, FN, FR-α, G250, GAGE-1, GAGE-2, GAGE-3, GAGE-4, GAGE-5, GAGE-6, GAGE-7B, GAGE-8, GD2, GD3, GnT-V, Gp100, gp75, GPC3, GPC-2, GUCY2C, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, L1-CAM, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, and K-light chain. In some embodiments, the second antigen is a receptor or ligand expressed on the cell that can interact with the T cell. In some embodiments, the second antigen is selected from a group consisting of CD40, CLL1, FLT3, FLT3L, 4-1BB, 4-1BBL, GITR, GITRL, CD27, CD70, OX40, OX40L, PD-1, PD-L1, PD-L2, Galectin-9, B7-H3, B7-H4, ICAM1, ICOS, ICOSL, CD30, CD30L, TIM1, TIM3, TIM4, SEMA4A, CD155, TIGIT, CD160, CD28, CD80, CD86, CTLA4, LAG3, LFA-1, LTβR, and HVEM.

In some embodiments, the second antigen is LTβR. In some embodiments, the domain capable of binding to the second antigen comprises a LTα or variant thereof. In some embodiments, the LTα or variant thereof comprises an amino acid sequence of SEQ ID NO: 7, SEQ ID NO: 8, or SEQ ID NO: 9. In other embodiments, the domain capable of binding to the second antigen comprises a LTβ or variant thereof. In some embodiments, the LTβ or variant thereof comprises an amino acid sequence of SEQ ID NO: 10, SEQ ID NO: 11, or SEQ ID NO: 12. In yet other embodiments, the domain capable of binding to the second antigen comprises a LTα or variant thereof and a LTβ or variant thereof. In some embodiments, the LTα or variant thereof comprises an amino acid sequence of SEQ ID NO: 7, SEQ ID NO: 8, or SEQ ID NO: 9. In some embodiments, the LTβ or variant thereof comprises an amino acid sequence of SEQ ID NO: 10, SEQ ID NO: 11, or SEQ ID NO: 12. In other embodiments, the domain comprises an amino acid sequence of SEQ ID NO: 13, SEQ ID NO: 14, SEQ ID NO: 15, SEQ ID NO: 16, or SEQ ID NO: 20.

In some embodiments, the second antigen is HVEM. In some embodiments, the domain capable of binding to the second antigen comprises a LIGHT (TNFSF14) or variant thereof. In some embodiments, the LIGHT or variant thereof comprises an amino acid sequence of SEQ ID NO: 17.

In other embodiments, the second antigen is CD40. In some embodiments, the domain capable of binding to the second antigen comprises an antibody or fragment thereof that binds CD40. In some embodiments, the antibody or fragment thereof that binds CD40 comprises an amino acid sequence of SEQ ID NO: 21. In some embodiments, the domain capable of binding to the second antigen comprises an amino acid sequence of SEQ ID NO: 24.

In some embodiments, the second antigen is CLL1. In some embodiments, the domain capable of binding to the second antigen comprises an antibody or fragment thereof that binds CLL1. In some embodiments, the antibody or fragment thereof that binds CLL1 comprises an amino acid sequence of SEQ ID NO: 22 or SEQ ID NO: 23.

In another aspect, provided herein is a pharmaceutical composition, comprising the polypeptide, the vector, or the CAR-T cell provided herein, and a pharmaceutically acceptable excipient.

In another aspect, provided herein is a method for treating a disease or disorder in a subject comprising administering to the subject a therapeutically effective amount of the CAR-T cell, or the pharmaceutical composition provided herein. In some embodiments, the disease or disorder is cancer. In some embodiments, the cancer is blood cancer. In other embodiments, the cancer is solid tumor cancer. In some embodiments, the subject is a human subject in need thereof.

In yet another aspect, provided herein is a composition comprising a CAR-T cell and an APC, wherein the CAR-T cell expresses (a) a CAR comprising (i) an extracellular domain, (ii) a transmembrane domain, and (iii) an intracellular domain; and (b) a domain, and wherein the extracellular domain binds a first antigen expressed on the surface of the target cell, and the domain binds a second antigen expressed on the surface of the APC.

### 4. BRIEF DESCRIPTION OF THE FIGS

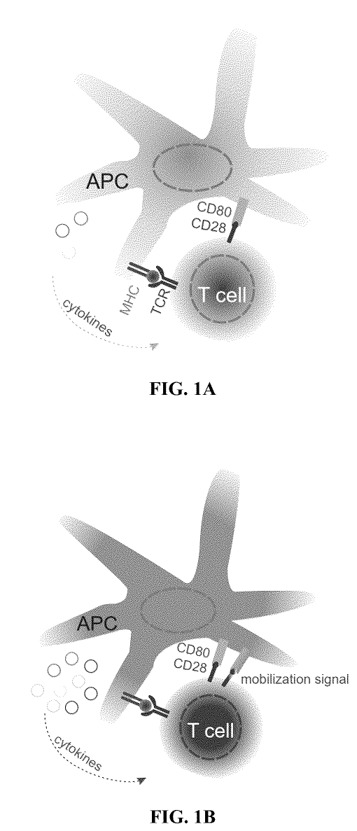

FIGS. 1A-1B show a schematic illustration of bi-directional mutual beneficial interaction between endogenous APC cells and adoptive transferred T cells by a mobilization TALE strategy provided herein.

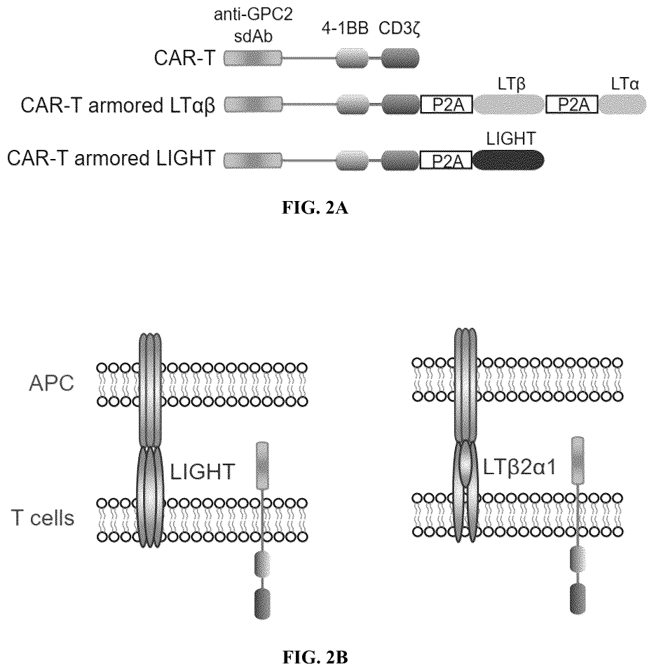

FIG. 2A shows a schematic illustration of molecular design of LTα/β and LIGHT derived TALE strategy. FIG. 2B shows consequent induction of physical proximity between T and APC cells.

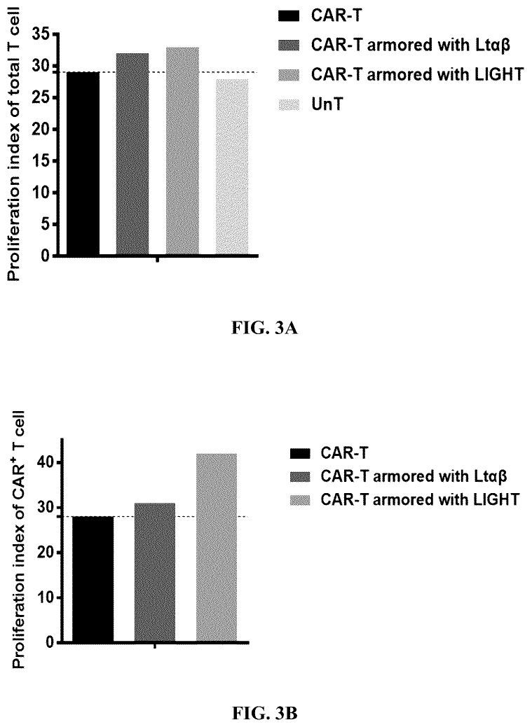

FIGS. 3A-3B show that LTα/β and LIGHT derived TALE strategy promotes homeostatic proliferation of total T cells (FIG. 3A) and CAR⁺ T cells (FIG. 3B). Monocyte induced homeostatic proliferation of T cells was calculated and is presented as proliferation index.

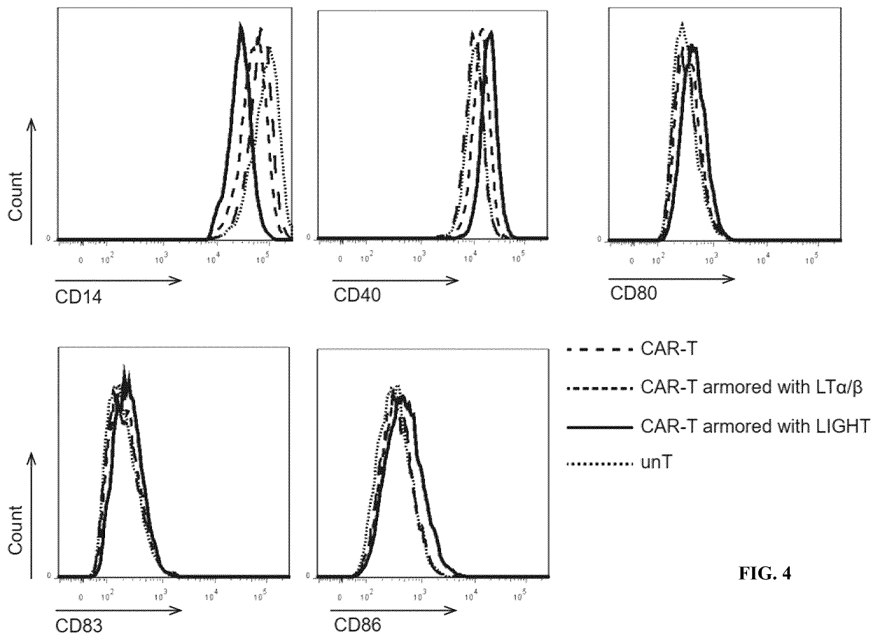

FIG. 4 shows flow cytometry analysis of monocyte activation induced by CAR-T cells armored with LTα/β and LIGHT derived TALE. Activation of monocyte is reflected as down-regulation of CD14 and upregulation of CD40, CD80, CD83 and CD86.

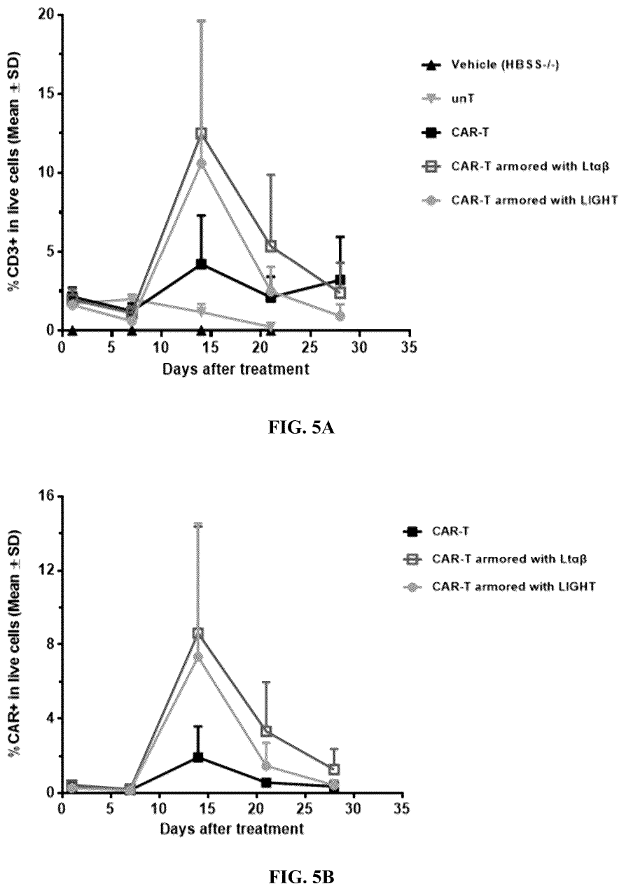

FIGS. 5A-5B show LTα/β and LIGHT derived TALEs substantially promote expansion of CAR-T cells in vivo. Quantification of CD3 T cell % and CAR⁺ T cell in live cell in the peripheral blood is presented in FIG. 5A and FIG. 5B, respectively.

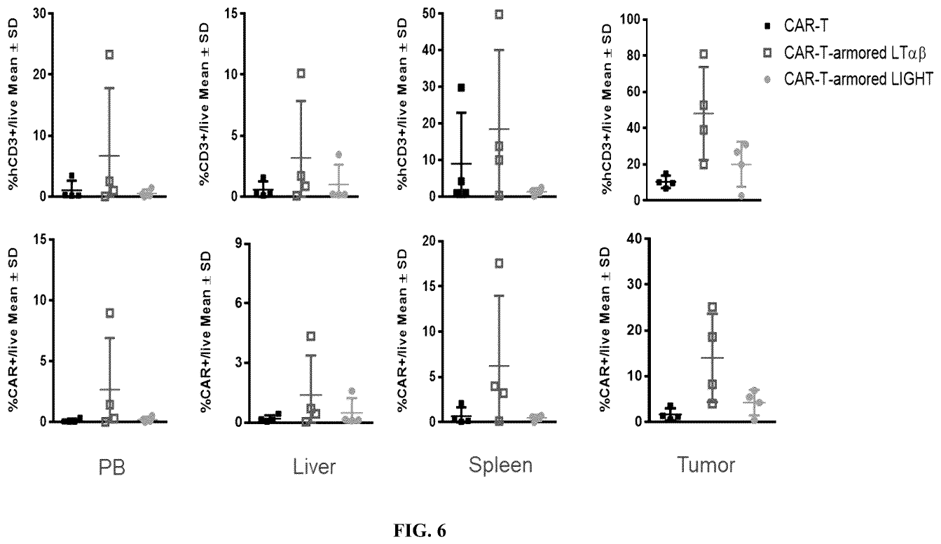

FIG. 6 shows LTα/β and LIGHT derived TALEs substantially promote CAR-T abundance in vivo in organs and tumors. Results are presented as a quantification of CD3 T cell % and CAR⁺ T cell in live cell.

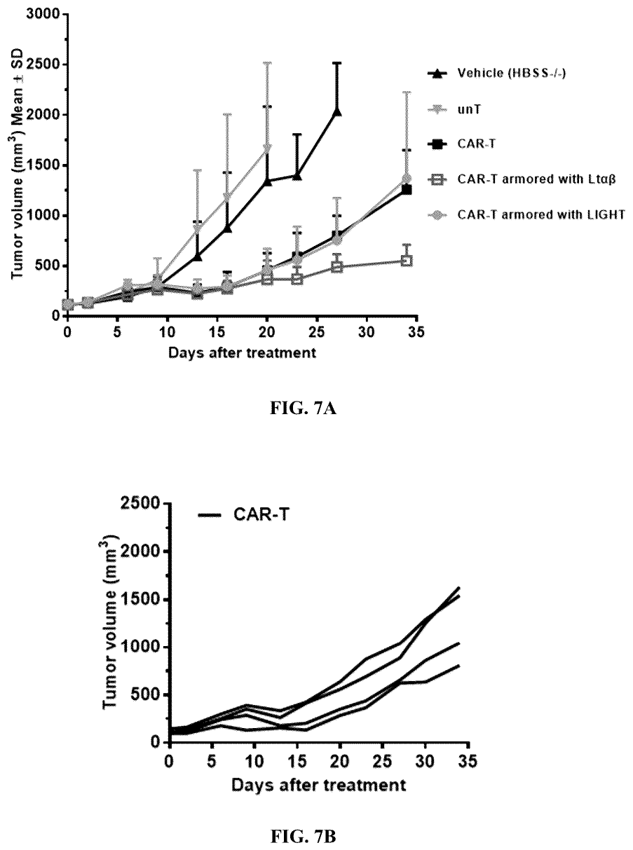

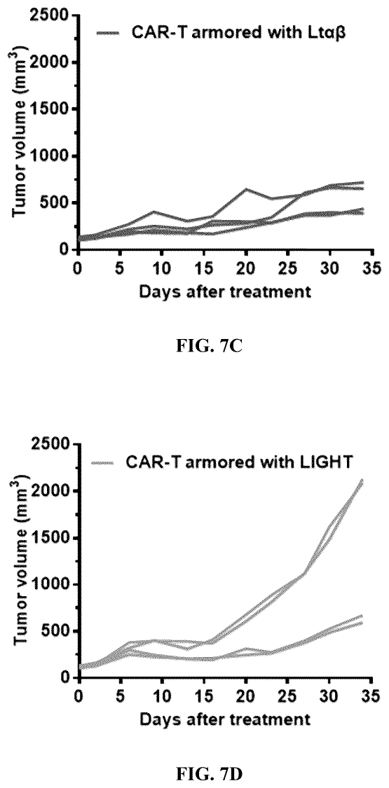

FIGS. 7A-7D show LTα/β and LIGHT derived TALEs substantially enhance anti-tumor activity of CAR-T cells in vivo. The summary of growth curve of different groups (FIG. 7A) and detail growth curve of each mice treated with different types of CAR-T cells (FIG. 7B, FIG. 7C, and FIG. 7D) are presented.

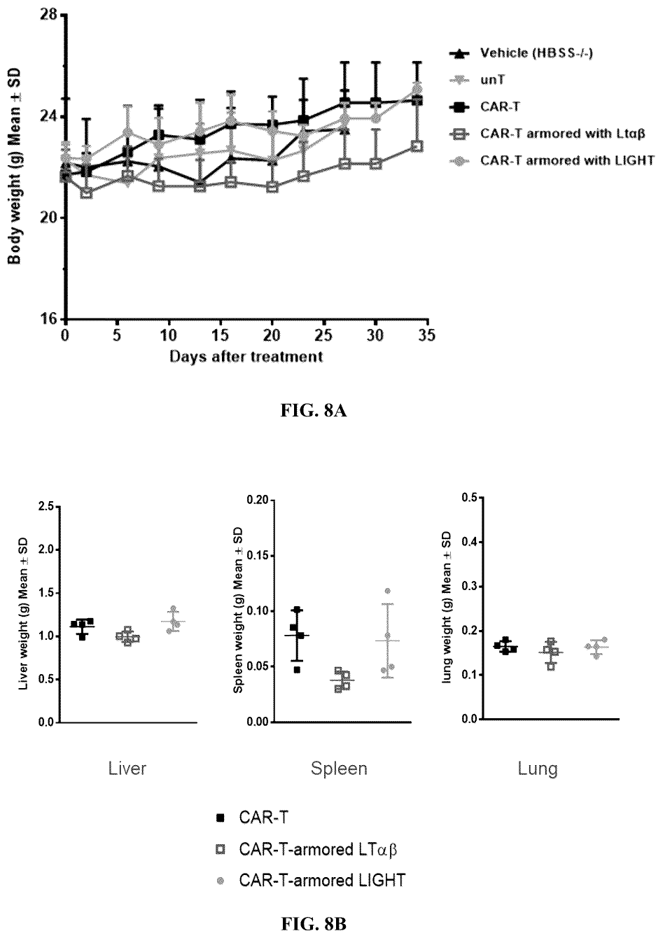

FIGS. 8A-8B show LTα/β and LIGHT derived TALEs are well-tolerated in vivo. Changes of body weight (FIG. 8A) as well as organ weight in the end point (FIG. 8B) demonstrate no clear differences between mice treated with conventional CAR-T cells and TALE armored CAR-T cells.

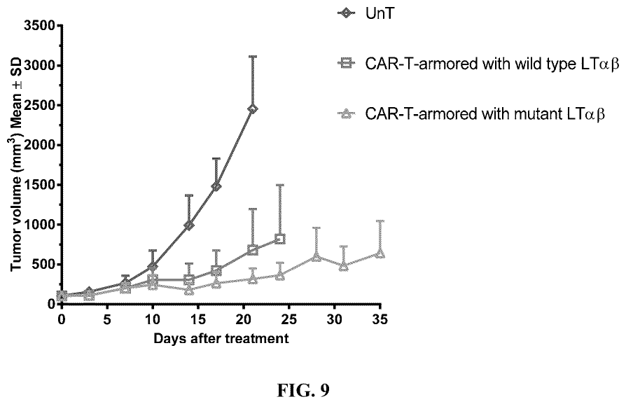

FIG. 9 shows anti-tumor efficacy is more pronounced in CAR-T cell armored engineered mutant LTα/β TALE as compared to CAR-T cells armored with wild-type LTα/β.

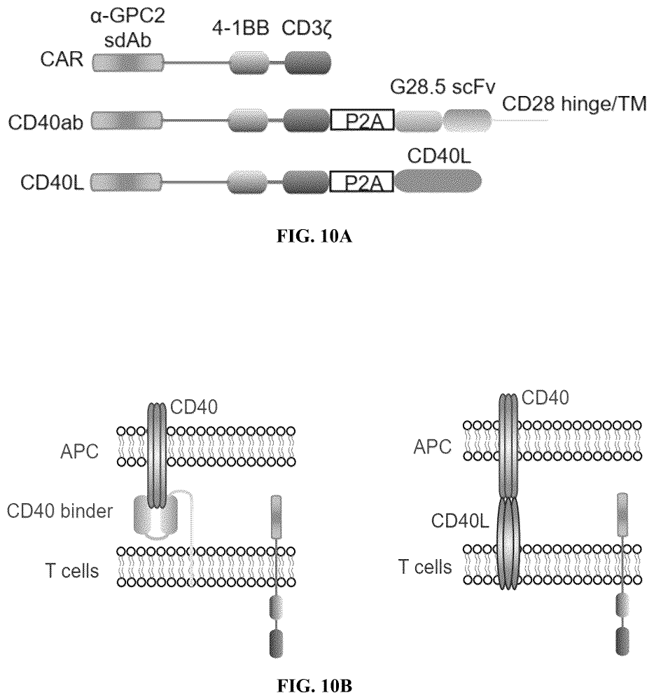

FIG. 10A shows a schematic illustration of molecular design of CD40 based TALE strategy. FIG. 10B shows consequent induction of physical proximity between T and APC cells.

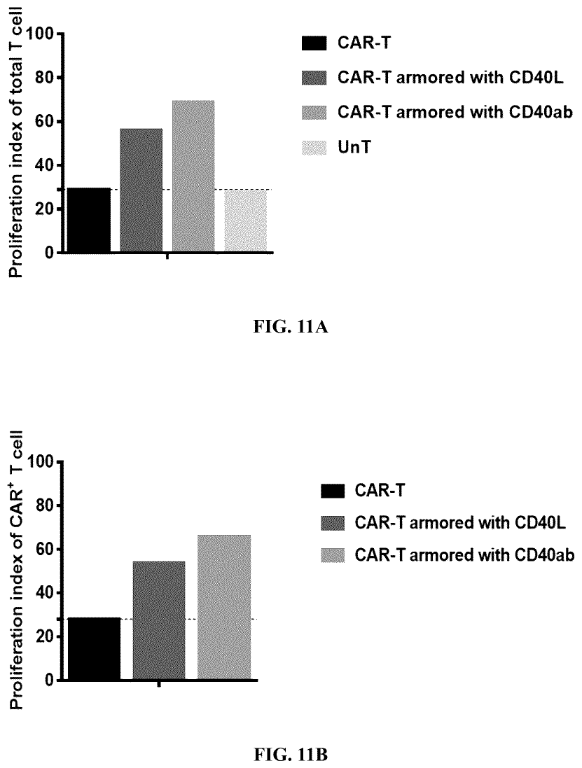

FIGS. 11A-11B show CD40 based TALE strategy promotes homeostatic proliferation of total T cells (FIG. 11A) and CAR⁺ T cells (FIG. 11B). Monocyte induced homeostatic proliferation of T cells was calculated and is presented as proliferation index.

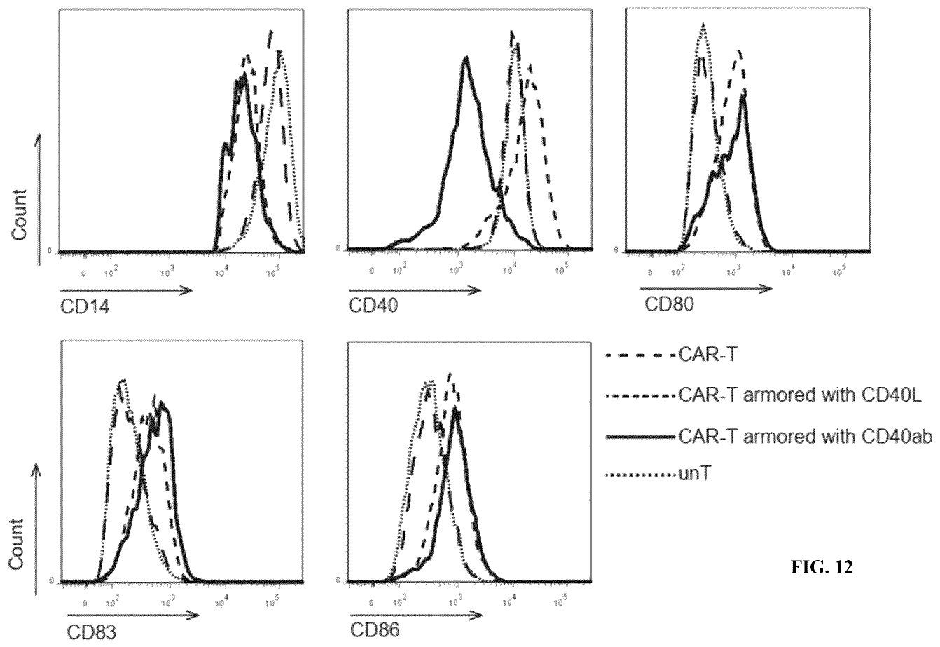

FIG. 12 shows flow cytometry analysis of monocyte activation induced by CAR-T cells armored with CD40 based TALE. Activation of monocyte is reflected as down-regulation of CD14 and upregulation of CD40, CD80, CD83 and CD86.

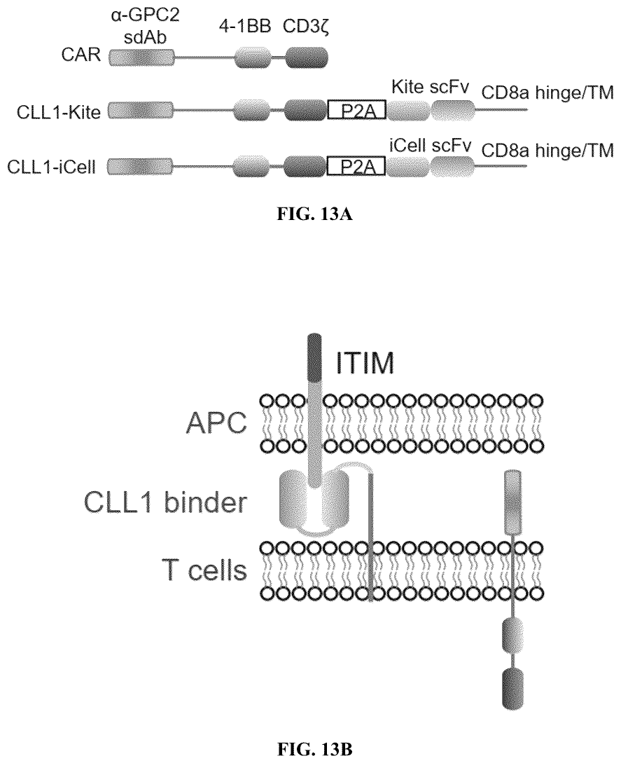

FIG. 13A shows a schematic illustration of molecular design of CLL1 based TALE strategy. FIG. 13B shows consequent induction of physical proximity between T and APC cells.

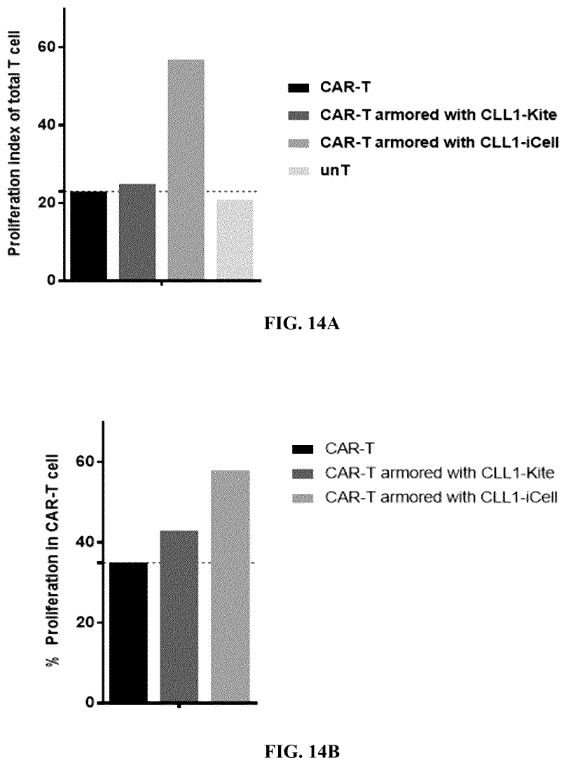

FIGS. 14A-14B show CLL1 based TALE strategy promotes homeostatic proliferation of total T cells (FIG. 14A) and CAR⁺ T cells (FIG. 14B). Monocyte induced homeostatic proliferation of T cells was calculated and is presented as proliferation index.

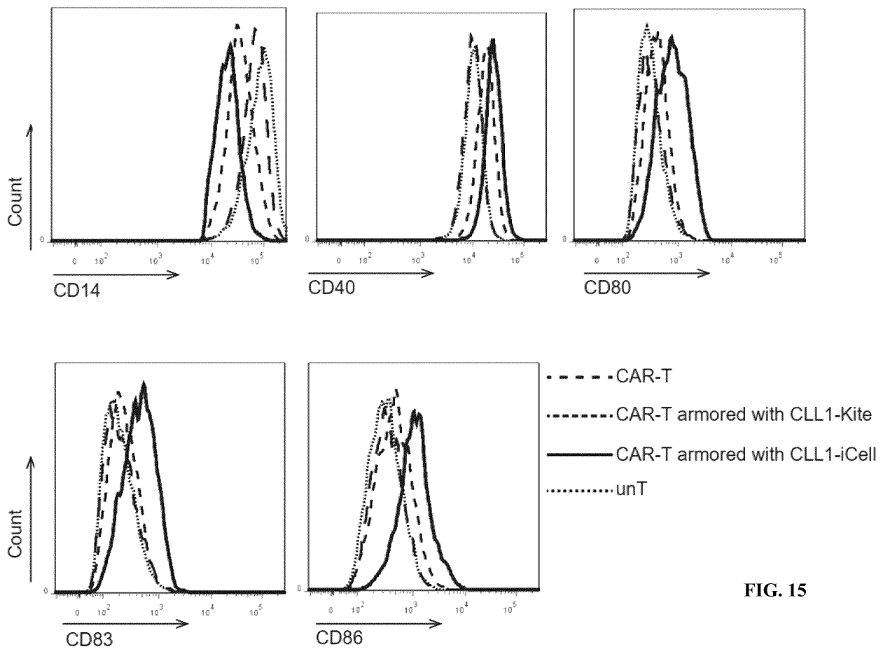

FIG. 15 shows flow cytometry analysis of monocyte activation induced by CAR-T cells armored with CLL1 based TALE. Activation of monocyte is reflected as down-regulation of CD14 and upregulation of CD40, CD80, CD83 and CD86.

### 5. DETAILED DESCRIPTION

The present disclosure is based, in part, on the surprising finding of improved expansion and function of CAR-T cells by using a T cell and APC cell engager (TALE).

Adoptive transfer of T cells, e.g., CAR-T cells, represents an emerging promising therapeutic against cancer. However, the proliferative capacity of genetically engineered T cells is generally limited, known as exhaustion, due to lack of full spectrum of APC co-stimulations as well as APC derived cytokines. Consequently, the efficacy of CAR-T or TCR-T cells against tumors is greatly impaired due to lack of activated APC in the tumor microenvironment.

To improve the efficacy of CAR-T cells against tumors, several strategies have been tested in preclinical models, such as CAR-T cells engineered with secretion of pro-inflammatory cytokines (see, e.g., Adachi et at, Nature Biotechnology, 36(4): 346-51 (2018); Hurton et al., Proceedings of the National Academy of Sciences, 113(48): E7788-E97 (2016); Ma et al., Nature Biotechnology, 38(4): 448-59 (2020)), armored with dominant negative inhibitory signal receptors (see, e.g., Kloss el al., Molecular Therapy, 26(7): 1855-66 (2018)), or utilization of multi-co-stimulatory signals (see, e.g., Tokarew el al., British Journal of Cancer, 120(1):26-37 (2019)). However, these approaches do not fully mimic natural APC-T cell interaction with multi-layer feedbacks from APC cells to T cells.

Recent studies using a small molecule, such as AMPH-FITC-mediated labeling of APC, can substantially increase the proliferation as well as anti-tumor efficacy of a FITC-tandem CAR-T cells (see Ma et al., Science, 365(6449): 162-8 (2019)). Likewise, liposome-mediated TAA expression in APC has also been explored to boost the expansion and anti-tumor efficacy of conventional CAR-T cells in vivo (see Reinhard et al., Science, 367(6476): 446-53 (2020)). These studies aimed at directing CAR-T cells to APC cells via a second target or even the same TAA to facilitate APC cells-induced activation and expansion of CAR-T cells. However, activation of CAR-T cells generally lead to cytotoxicity towards target/TAA expression cells, which suggest that APC will most likely be eliminated by CAR-T cells as a consequence of CAR-T cell activation. This side-effect of CAR-T cells to labeled APC cells may limit the clinical applications of these approaches.

The present disclosure takes advantage of a TALE-induced physical proximity between T cells and APC to boost a stable interaction between adoptive transferred T cells and APC. As shown in Section 6 below, the TALE provided herein can stimulate a bi-directional mutual beneficial interaction between these two cell types. Mutual beneficial interaction between T cells and APC promotes APC-induced T cell proliferation as well as T cell-mediated activation of APCs. As such, a TALE induces a positive feedback loop between T cells and APCs. The compositions of matters and methods provided herein based on TALE represent an effective strategy to shape the tumor inflammatory environment with an aim at improving the proliferation of CAR-T cells and enhancing cytotoxicity against tumors. Importantly, the results (see Section 6 below) indicate that the bi-directional mutual beneficial interaction can be induced by TALEs targeting both activating receptors/ligands and inhibitory receptors/ligands expressed on APCs. More specifically, the present findings suggest that any APC receptors, such as LTβR, HVEM, CD40 and CLL1, irrespective inflammatory promoting or inhibiting, can be a potent target to increase the function of T cells both in vitro and in vivo. In other words, a TALE binding to either functional or structural receptors/ligands on APCs can all be utilized according to the present disclosure.

Furthermore, increased proliferation of adoptively transferred CAR-T cells positively correlates with enhanced efficacy in vivo. Thus, the present compositions and methods can improve the CAR-T therapy efficacy by promoting CAR-T cell proliferation, thereby increasing its abundance in peripheral as well as in the vicinity of tumor, as demonstrated in Section 6 below, e.g., by showing that the bi-directional mutual beneficial interaction between APCs and CAR-T cell promotes APC-induced CAR-T cell proliferation, CAR-T cell-induced APC maturation, and matured APC feedforward to CAR-T with activated co-stimulatory signals as well as pro-inflammatory cytokines.

In sum, the present disclosure present compositions and methods that can substantially promote the proliferation as well as anti-tumor efficacy of CAR-T cells, and shape the inflammatory tumor microenvironment by promoting the maturation of APCs within the vicinity of tumor.

### 5.1. Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies), as described below. An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515, and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

An “antigen” is a structure to which a binding polypeptide or polypeptide complex (such as an antibody or fragment thereof, a ligand, a receptor, etc.) can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Single domain antibody” or “sdAb” as used herein refers to a single monomeric variable antibody domain and which is capable of antigen binding. Single domain antibodies include V_{H}H domains as described herein. Examples of single domain antibodies include, but are not limited to, antibodies naturally devoid of light chains such as those from Camelidae species (e.g., llama), single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. For example, a single domain antibody can be derived from antibodies raised in Camelidae species, for example in camel, llama, dromedary, alpaca and guanaco, as described herein. Other species besides Camelidae may produce heavy chain antibodies naturally devoid of light chain; V_{H}Hs derived from such other species are within the scope of the disclosure. In some embodiments, the single domain antibody (e.g., V_{H}H) provided herein has a structure of FR1-CDR1-FR2-CDR2-FR3-CDR3-FR4. Single domain antibodies may be genetically fused or chemically conjugated to another molecule (e.g., an agent) as described herein. Single domain antibodies may be part of a bigger binding molecule (e.g., a multispecific antibody or a chimeric antigen receptor).

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (koff) to association rate (kon) of a binding molecule (e.g., an antibody) to a monovalent antigen (koff/kon) is the dissociation constant KD, which is inversely related to affinity. The lower the KD value, the higher the affinity of the antibody. The value of KD varies for different complexes of antibody and antigen and depends on both kon and koff. The dissociation constant KD for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen can be identified, for example, by immunoassays, Octet®, Biacore®, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as radioimmunoassay (RIA) and enzyme linked immunosorbent assay (ELISA). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by FACS analysis or RIA. A binding molecule or antigen binding domain that binds to an antigen includes one that is capable of binding the antigen with sufficient affinity such that the binding molecule is useful, for example, as a therapeutic and/or diagnostic agent in targeting the antigen. In certain embodiments, a binding molecule or antigen binding domain that binds to an antigen has a dissociation constant (KD) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species.

In certain embodiments, the binding molecules or antigen binding domains can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55). Chimeric sequences may include humanized sequences.

In certain embodiments, the binding molecules or antigen binding domains can comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody) such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 321:522-25 (1986); Riechmann et al., Nature 332:323-29 (1988); Presta, Curr. Op. Struct. Biol. 2:593-96 (1992); Carter et al., Proc. Natl. Acad. Sci. USA 89:4285-89 (1992); U.S. Pat. Nos. 6,800,738; 6,719,971; 6,639,055; 6,407,213; and 6,054,297.

In certain embodiments, the binding molecules or antigen binding domains can comprise portions of a “fully human antibody” or “human antibody,” wherein the terms are used interchangeably herein and refer to an antibody that comprises a human variable region and, for example, a human constant region. The binding molecules may comprise a single domain antibody sequence. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 227:381 (1991); Marks et al., J. Mol. Biol. 222:581(1991)) and yeast display libraries (Chao et al., Nature Protocols 1: 755-68 (2006)). Also available for the preparation of human monoclonal antibodies are methods described in Cole et al., Monoclonal Antibodies and Cancer Therapy 77 (1985); Boerner et al., J. Immunol. 147(1):86-95 (1991); and van Dijk and van de Winkel, Curr. Opin. Pharmacol. 5: 368-74(2001). Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 6(5):561-66 (1995); Bruggemann and Taussing, Curr. Opin. Biotechnol. 8(4):455-58 (1997); and U.S. Pat. Nos. 6,075,181 and 6,150,584 regarding XENOMOUSE™ technology). See also, for example, Li et al., Proc. Natl. Acad. Sci. USA 103:3557-62 (2006) regarding human antibodies generated via a human B-cell hybridoma technology.

In certain embodiments, the binding molecules or antigen binding domains can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al., Nucl. Acids Res. 20:6287-6295 (1992)) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the binding molecules or antigen binding domains can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts or well-known post-translational modifications such as amino acid iomerizatio or deamidation, methionine oxidation or asparagine or glutamine deamidation, each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., Nature 256:495 (1975), or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352:624-28 (1991) and Marks et al., J. Mol. Biol. 222:581-97 (1991), for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5th ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, J. Mol. Biol. 196:901-17 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dubel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., Dev. Comp. Immunol. 27(1):55-77 (2003)). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-70 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are exemplified in Table 1 below.

TABLE 1
Exemplary CDRs According to Various Numbering Systems

| Loop | Kabat | AbM | Chothia | Contact | IMGT |
|---|---|---|---|---|---|
| CDR L1 | L24--L34 | L24--L34 | L26--L32 or | L30--L36 | L27--L38 |
| | | | L24--L34 | | |
| CDR L2 | L50--L56 | L50--L56 | L50--L52 or | L46--L55 | L56--L65 |
| | | | L50--L56 | | |
| CDR L3 | L89--L97 | L89--L97 | L91--L96 or | L89--L96 | L105--L117 |
| | | | L89--L97 | | |
| CDR H1 | H31--H35B | H26--H35B | H26--H32 . . . 34 | H30--H35B | H27--H38 |
| | (Kabat | | | | |
| | Numbering) | | | | |
| CDR H1 | H31--H35 | H26--H35 | H26--H32 | H30--H35 | |
| | (Chothia | | | | |
| | Numbering) | | | | |
| CDR H2 | H50--H65 | H50--H58 | H53--H55 or | H47--H58 | H56--H65 |
| | | | H52--H56 | | |
| CDR H3 | H95--H102 | H95--H102 | H96--H101 or | H93--H101 | H105--H117 |
| | | | H95--H102 | | |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, Chothia, or Contact method. In other cases, the particular amino acid sequence of a CDR is given. It should be noted CDR regions may also be defined by a combination of various numbering systems, e.g., a combination of Kabat and Chothia numbering systems, or a combination of Kabat and IMGT numbering systems. Therefore, the term such as “a CDR as set forth in a specific VH” includes any CDR1 as defined by the exemplary CDR numbering systems described above, but is not limited thereby. Once a variable region (e.g., a VH or VL) is given, those skilled in the art would understand that CDRs within the region can be defined by different numbering systems or combinations thereof.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies (e.g., single domain antibodies), diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substitution, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody comprising a single domain antibody sequence) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

“Chimeric antigen receptor” or “CAR” as used herein refers to genetically engineered receptors, which can be used to graft one or more antigen specificity onto immune effector cells, such as T cells. Some CARs are also known as “artificial T-cell receptors,” “chimeric T cell receptors,” or “chimeric immune receptors.” In some embodiments, the CAR comprises an extracellular antigen binding domain specific for one or more antigens (such as tumor antigens), a transmembrane domain, and an intracellular signaling domain of a T cell and/or other receptors. “CAR-T cell” refers to an α/β T cell or γ/δ T cell that expresses a CAR. The present disclosure can be used with any CAR, including but not limited to what are referred to as first-generation, second-generation, third-generation, and/or “armored” CARs. All forms of CARs can be suitably used in the present disclosure, including but not limited to single CAR, tandem CAR, or dual CAR, or a combination thereof.

Single CAR

A chimeric molecule that includes a single antigen binding domain (such as sdAb or scFv), transmembrane domain, and an intracellular signaling domain, such as a signaling domain from a T cell receptor (e.g., CD3ζ). Typically, single CARs may comprise a monospecific antigen-binding moiety targeting a tumor antigen, such as GPC2, CD19 or BCMA, a transmembrane domain, and an intracellular domain.

Tandem CAR and Dual CAR

Tandem CAR includes more than one antigen-binding portions (such as 2, 3, 4, 5, or 6 sdAb or scFv) in tandem. Typically, tandem CARs may contain monospecific, bivalent antigen-binding moiety, e.g., two identical V_{H}H domains binding GPC3, or multi-specific, e.g., bispecific bivalent, antigen-binding moiety, e.g., two different V_{H}H domains binding GPC3 or one V_{H}H domain binding GPC3 and the other V_{H}H domain binding a molecule other than GPC3, a transmembrane domain, and an intracellular domain. In another aspect, the CAR of the present disclosure can include a tandem CAR having an extracellular antigen recognition domain including a first binding domain and a second binding domain, wherein the first binding domain fuses to the second binding domain optionally via a linker.

In some embodiments, the CAR used in the present disclosure is a tandem CAR which comprises: more than one antigen-binding portions (e.g., single domain antibody (sdAb)) that target different epitopes on one or more antigens, such as a tumor antigen, a transmembrane domain, and/or an intracellular signaling domain.

Dual CAR can be a combination of any two CARs, in which each of a first CAR and a second CAR can be a single CAR or a tandem CAR, i.e., single CAR/single CAR, single CAR/tandem CAR, or tandem CAR/tandem CAR. The levels of dual CAR T cell signaling can be regulated by manipulating the intracellular domains of each first and second CARs. For example, the intracellular domains of each of the first CAR and the second CAR can contain a co-stimulatory domain, such as CD28, 4-1BB (CD137), ICOS, OX40 (CD134), CD27, and/or DAP10, and/or a signaling domain from a T cell receptor, such as a signaling domain from a T cell receptor (e.g., CD3ζ). For example, dual CAR of the present disclosure can include a first CAR and a second CAR each having an intracellular domain containing a co-stimulatory domain and a signaling domain from a T cell receptor. Thus, when dual CAR bind antigens (e.g., bispecific), the T cell signals can be transmitted through two signaling domains from a T cell receptor. Dual CAR of the present disclosure can also include a first CAR having an intracellular domain containing a co-stimulatory domain and a signaling domain from a T cell receptor and a second CAR having an intracellular domain containing a co-stimulatory domain. Thus, when dual CAR bind antigens (e.g., bispecific), the T cell signals can be transmitted through the signaling domain from a T cell receptor of the first CAR.

In some embodiments of the present disclosure, the tandem CAR or dual CAR targets the same tumor antigen, for example, they can target different epitopes on the same tumor antigen, such as different epitopes of BCMA, different epitopes on CD19, different epitope on DLL3, or different epitopes on GPC2. In some embodiments, the tandem CAR or dual CAR targets different tumor antigens, such as BCMA, CD19, and/or GPC2.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a binding molecule of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding a single domain antibody or an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule. Specifically, an “isolated” nucleic acid molecule encoding a CAR described herein is a nucleic acid molecule that is identified and separated from at least one contaminant nucleic acid molecule with which it is ordinarily associated in the environment in which it was produced.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, the term “operatively linked,” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “vector” refers to a substance that is used to carry or include a nucleic acid sequence, including for example, a nucleic acid sequence encoding a binding molecule (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the term “autologous” is meant to refer to any material derived from the same individual to whom it is later to be re-introduced into the individual.

“Allogeneic” refers to a graft derived from a different individual of the same species.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, P A, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain a binding molecule (e.g., an antibody), for example, in isolated or purified form, together with a suitable amount of excipients.

The term “effective amount” or “therapeutically effective amount” as used herein refers to the amount of a single domain antibody or a therapeutic molecule comprising an agent and the single domain antibody or pharmaceutical composition provided herein which is sufficient to result in the desired outcome.

The terms “subject” and “patient” may be used interchangeably. As used herein, in certain embodiments, a subject is a mammal, such as a non-primate or a primate (e.g., human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., diabetes or a cancer).

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.2. Polypeptides Comprising a Chimeric Antigen Receptor and an Additional Binding Domain

Provided herein, in one aspect, is a polypeptide comprising at least two regions—a chimeric antigen receptor (CAR) capable of binding to a first antigen and a domain capable of binding to a second antigen (e.g., a receptor or a ligand) expressed on the surface of a cell (e.g., an APC), wherein the two regions are linked by a linker. In some embodiments, the linker is cleavable in cells (e.g., a self-cleaving peptide linker), so that the two regions are expressed as two individual domains on the surface of the engineered immune cell (e.g., a T cell), when the immune cell (e.g., a T cell) is transfected with a polynucleotide encoding such a polypeptide. The CAR expressed on the engineered immune cell can bind the antigen (e.g., a tumor antigen) presented by an antigen presenting cell (e.g., an APC); and the domain capable of binding to a receptor or a ligand on the surface of the antigen presenting cell (e.g., an APC) provides an additional physical interaction between the engineered immune cell (e.g., CAR-T cell) with the antigen presenting cell (e.g., APC).

Thus, in certain embodiments, provided herein is a polypeptide comprising (a) a CAR comprising (i) an extracellular domain capable of binding to a first antigen, (ii) a transmembrane domain, and (iii) an intracellular domain; and (b) a domain capable of binding to a second antigen expressed on the surface of a cell that can interact with a T cell, wherein the CAR and the domain are fused by a peptide linker. The CAR and the domain can be in any order in the polypeptide provided herein. In some embodiments, the CAR is at the N-terminus of the domain. In other embodiments, the CAR is at the C-terminus of the domain. The CAR and the additional binding domain in the present polypeptides are described in more detail below.

Furthermore, all the description in the present disclosure also applies to engineered T cell receptor (TCR) in addition to CARs. Thus, in another aspect provided herein is a polypeptide comprising at least two regions—a TCR (e.g., engineered TCR) capable of binding to a first antigen and a domain capable of binding to a second antigen (e.g., a receptor or a ligand) expressed on the surface of a cell (e.g., an APC), wherein the two regions are linked by a linker. In some embodiments, the linker is cleavable in cells (e.g., a self-cleaving peptide linker), so that the two regions are expressed as two individual domains on the surface of the engineered immune cell (e.g., a T cell), when the immune cell (e.g., a T cell) is transfected with a polynucleotide encoding such a polypeptide. The TCR expressed on the engineered immune cell can bind the antigen (e.g., a tumor antigen) presented by an antigen presenting cell (e.g., an APC); and the domain capable of binding to a receptor or a ligand on the surface of the antigen presenting cell (e.g., an APC) provides an additional physical interaction between the engineered immune cell (e.g., TCR-T cell) with the antigen presenting cell (e.g., APC). In certain embodiments, provided herein is a polypeptide comprising (a) a TCR capable of binding to a first antigen, and (b) a domain capable of binding to a second antigen expressed on the surface of a cell that can interact with a T cell, wherein the TCR and the domain are fused by a peptide linker. The TCR and the domain can be in any order in the polypeptide provided herein. In some embodiments, the TCR is at the N-terminus of the domain. In other embodiments, the TCR is at the C-terminus of the domain.

5.2.1. Chimeric Antigen Receptors

In some embodiments, the CAR provided herein comprises a polypeptide comprising: (a) an extracellular antigen binding domain; (b) a transmembrane domain; and (c) an intracellular signaling domain, each of which and additional regions are described in more detail below.

### Extracellular Antigen Binding Domain

The extracellular antigen binding domain of the CARs described herein comprises one or more antigen binding domains. In some embodiments, the extracellular antigen binding domain of the CAR provided herein is mono-specific. In other embodiments, the extracellular antigen binding domain of the CAR provided herein is multispecific. In other embodiments, the extracellular antigen binding domain of the CAR provided herein is multivalent. In some embodiments, the extracellular antigen binding domain comprises two or more antigen binding domains which are fused to each other directly via peptide bonds, or via peptide linkers.

In some embodiments, the extracellular antigen binding domain comprises an antibody or a fragment thereof. For example, the binding domain may be derived from monoclonal antibodies (including agonist, antagonist, neutralizing antibodies, full length or intact monoclonal antibodies), antibody with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies). An antibody can be human, humanized, chimeric and/or affinity matured, as well as an antibody from other species, for example, mouse, rabbit, llama, etc. In some embodiments, the antibody include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515, and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

In a specific embodiment, the extracellular antigen binding domain of the present CARs comprise a single-chain Fv (sFv or scFv). scFvs are antibody fragments that comprise the VH and VL antibody domains connected into a single polypeptide chain. Preferably, the scFv polypeptide further comprises a polypeptide linker between the VH and VL domains which enables the sFv to form the desired structure for antigen binding. See Pluckthun in The Pharmacology of Monoclonal Antibodies, vol. 113, Rosenburg and Moore eds., Springer-Verlag, New York, pp. 269-315 (1994).

In another specific embodiment, the extracellular antigen binding domain of the present CARs comprises one or more single domain antibodies (sdAbs). The sdAbs may be of the same or different origins, and of the same or different sizes. Exemplary sdAbs include, but are not limited to, heavy chain variable domains from heavy-chain only antibodies (e.g., V_{H}H or V_{NAR}), binding molecules naturally devoid of light chains, single domains (such as V_{H} or V_{L}) derived from conventional 4-chain antibodies, humanized heavy-chain only antibodies, human single domain antibodies produced by transgenic mice or rats expressing human heavy chain segments, and engineered domains and single domain scaffolds other than those derived from antibodies. Any sdAbs known in the art or developed by the present disclosure, including the single domain antibodies described above in the present disclosure, may be used to construct the CARs described herein. The sdAbs may be derived from any species including, but not limited to mouse, rat, human, camel, llama, lamprey, fish, shark, goat, rabbit, and bovine. Single domain antibodies contemplated herein also include naturally occurring single domain antibody molecules from species other than Camelidae and sharks.

In some embodiments, the sdAb is derived from a naturally occurring single domain antigen binding molecule known as heavy chain antibody devoid of light chains (also referred herein as “heavy chain only antibodies”). Such single domain molecules are disclosed in WO 94/04678 and Hamers-Casterman, C. et al., Nature 363:446-448 (1993), for example. For clarity reasons, the variable domain derived from a heavy chain molecule naturally devoid of light chain is known herein as a V_{H}H to distinguish it from the conventional V_{H} of four chain immunoglobulins. Such a V_{H}H molecule can be derived from antibodies raised in Camelidae species, for example, camel, llama, vicuna, dromedary, alpaca and guanaco. Other species besides Camelidae may produce heavy chain molecules naturally devoid of light chain, and such V_{H}Hs are within the scope of the present disclosure. In addition, humanized versions of V_{H}Hs as well as other modifications and variants are also contemplated and within the scope of the present disclosure. In some embodiments, the sdAb is derived from a variable region of the immunoglobulin found in cartilaginous fish. For example, the sdAb can be derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov, Protein Sci. 14:2901-2909 (2005).

In some embodiments, naturally occurring V_{H}H domains against a particular antigen or target, can be obtained from (naïve or immune) libraries of Camelid V_{H}H sequences. Such methods may or may not involve screening such a library using said antigen or target, or at least one part, fragment, antigenic determinant or epitope thereof using one or more screening techniques known in the field. Such libraries and techniques are for example described in WO 99/37681, WO 01/90190, WO 03/025020 and WO 03/035694. Alternatively, improved synthetic or semi-synthetic libraries derived from (naïve or immune) V_{H}H libraries may be used, such as V_{H}H libraries obtained from (naïve or immune) V_{H}H libraries by techniques such as random mutagenesis and/or CDR shuffling, as for example described in WO 00/43507.

In some embodiments, the sdAb is recombinant, CDR-grafted, humanized, camelized, de-immunized and/or in vitro generated (e.g., selected by phage display). In some embodiments, the amino acid sequence of the framework regions may be altered by “camelization” of specific amino acid residues in the framework regions. Camelization refers to the replacing or substitution of one or more amino acid residues in the amino acid sequence of a (naturally occurring) VH domain from a conventional 4-chain antibody by one or more of the amino acid residues that occur at the corresponding position(s) in a V_{H}H domain of a heavy chain antibody. This can be performed in a manner known in the field, which will be clear to the skilled person. Such “camelizing” substitutions are preferably inserted at amino acid positions that form and/or are present at the VH-VL interface, and/or at the so-called Camelidae hallmark residues, as defined herein (see for example WO 94/04678, Davies and Riechmann FEBS Letters 339: 285-290 (1994); Davies and Riechmann, Protein Engineering 9 (6): 531-537 (1996); Riechmann, J. Mol. Biol. 259: 957-969 (1996); and Riechmann and Muyldermans, J. Immunol. Meth. 231: 25-38 (1999)).

In some embodiments, the sdAb is a human single domain antibody produced by transgenic mice or rats expressing human heavy chain segments. See, e.g., US20090307787, U.S. Pat. No. 8,754,287, US20150289489, US20100122358, and WO2004049794.

In some embodiments, the single domain antibodies are generated from conventional four-chain antibodies. See, for example, EP 0368684; Ward et al., Nature, 341 (6242): 544-6 (1989); Holt et al., Trends Biotechnol., 21(11):484-490 (2003); WO 06/030220; and WO 06/003388.

In some embodiments, the extracellular antigen binding domain comprises humanized antibodies or fragment thereof. A humanized antibody can comprise human framework region and human constant region sequences.

Humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. Nos. 6,407,213, 5,766,886, WO 93/17105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp):5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), each of which is incorporated by reference herein in its entirety.

Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36, by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (see, e.g., Kashmiri et al., 2005, Methods 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In some cases, the framework is derived from the consensus sequences of the most abundant human subclasses, VL6 subgroup I (VL6I) and VH subgroup III (VHIII). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., 2002, J. Immunol. 169:1119-25).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., 2007, Mol. Immunol. 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, 2005, Nat. Biotechnol. 23:1105-16; Dufner et al., 2006, Trends Biotechnol. 24:523-29; Feldhaus et al., 2003, Nat. Biotechnol. 21:163-70; and Schlapschy et al., 2004, Protein Eng. Des. Sel. 17:847-60).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, 1992, J. Mol. Biol. 224:487-99), or from the more limited set of target residues identified by Baca et al. (1997, J. Biol. Chem. 272:10678-84).

In FR shuffling, whole FRs are combined with the non-human CDRs instead of creating combinatorial libraries of selected residue variants (see, e.g., Dall'Acqua et al., 2005, Methods 36:43-60). The libraries may be screened for binding in a two-step process, first humanizing VL, followed by VH. Alternatively, a one-step FR shuffling process may be used. Such a process has been shown to be more efficient than the two-step screening, as the resulting antibodies exhibited improved biochemical and physicochemical properties including enhanced expression, increased affinity, and thermal stability (see, e.g., Damschroder et al., 2007, Mol. Immunol. 44:3049-60).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple subclasses with distinct human V-segment CDRs. Humaneering allows for isolation of antibodies that are 91-96% homologous to human germline gene antibodies (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., 1994, Protein Engineering 7:805-14; U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody. Such antibodies can comprise human constant region sequences, e.g., human light chain and/or heavy chain constant regions.

A deimmunized antibody is an antibody in which T-cell epitopes have been removed. Methods for making deimmunized antibodies have been described. See, e.g., Jones et al., Methods Mol Biol. 2009; 525:405-23, xiv, and De Groot et al., Cell. Immunol. 244:148-153(2006)). Deimmunized antibodies comprise T-cell epitope-depleted variable regions and human constant regions. Briefly, VH and VL of an antibody are cloned and T-cell epitopes are subsequently identified by testing overlapping peptides derived from the VH and VL of the antibody in a T cell proliferation assay. T cell epitopes are identified via in silico methods to identify peptide binding to human MHC class II. Mutations are introduced in the VH and VL to abrogate binding to human MHC class II. Mutated VH and VL are then utilized to generate the deimmunized antibody.

In certain embodiments, the extracellular antigen binding domain comprises multiple binding domains. In some embodiments, the extracellular antigen binding domain comprises multispecific antibodies or fragments thereof. In other embodiments, the extracellular antigen binding domain comprises multivalent antibodies or fragments thereof. The term “specificity” refers to selective recognition of an antigen binding protein for a particular epitope of an antigen. The term “multispecific” as used herein denotes that an antigen binding protein has two or more antigen-binding sites of which at least two bind different antigens. The term “valent” as used herein denotes the presence of a specified number of binding sites in an antigen binding protein. A full length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein.

Multispecific antibodies such as bispecific antibodies are antibodies that have binding specificities for at least two different antigens. Methods for making multispecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating multispecific antibodies (e.g., bispecific antibodies), see, for example, Bispecific Antibodies (Kontermann ed., 2011).

The antibodies of the present disclosure can be multivalent antibodies with two or more antigen binding sites (e.g., tetravalent antibodies), which can be readily produced by recombinant expression of nucleic acid encoding the polypeptide chains of the antibody. In certain embodiments, a multivalent antibody comprises (or consists of) three to about eight antigen binding sites. In one such embodiment, a multivalent antibody comprises (or consists of) four antigen binding sites. The multivalent antibody comprises at least one polypeptide chain (e.g., two polypeptide chains), wherein the polypeptide chain(s) comprise two or more variable domains. For instance, the polypeptide chain(s) may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (e.g., four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

In case there are multiple binding domains in the extracellular antigen binding domain of the present CARs, e.g., an extracellular antigen binding domain comprising multiple binding domains (e.g., multiple V_{H}Hs) in tandem. The various domains may be fused to each other via peptide linkers. In some embodiments, the domains are directly fused to each other without any peptide linkers. The peptide linkers may be the same or different. Each peptide linker may have the same or different length and/or sequence depending on the structural and/or functional features of the various domains. Each peptide linker may be selected and optimized independently. The length, the degree of flexibility and/or other properties of the peptide linker(s) used in the CARs may have some influence on properties, including but not limited to the affinity, specificity or avidity for one or more particular antigens or epitopes. In some embodiment, a peptide linker comprises flexible residues (such as glycine and serine) so that the adjacent domains are free to move relative to each other. For example, a glycine-serine doublet can be a suitable peptide linker.

The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a flexible linker. Exemplary flexible linkers include but not limited to glycine polymers (G)ₙ, glycine-serine polymers (including, for example, (GS)ₙ, (GSGGS)ₙ, (GGGS)ₙ, and (GGGGS)ₙ, where n is an integer of at least one), glycine-alanine polymers, alanine-serine polymers, and other flexible linkers known in the art. Exemplary peptide linkers are listed in the table below. Other linkers known in the art, for example, as described in WO2016014789, WO2015158671, WO2016102965, US20150299317, WO2018067992, U.S. Pat. No. 7,741,465, Colcher et al., J. Nat. Cancer Inst. 82:1191-1197 (1990), and Bird et al., Science 242:423-426 (1988) may also be included in the CARs provided herein, the disclosure of each of which is incorporated herein by reference.

In some embodiments, the extracellular antigen binding domain provided in the present CARs recognizes an antigen that acts as a cell surface marker on target cells associated with a special disease state. In some embodiments, the antigen is a tumor antigen. Tumors express a number of proteins that can serve as a target antigen for an immune response, particularly T cell mediated immune responses. The antigens targeted by the CAR may be antigens on a single diseased cell or antigens that are expressed on different cells that each contribute to the disease. The antigens targeted by the CAR may be directly or indirectly involved in the diseases.

Tumor antigens are proteins that are produced by tumor cells that can elicit an immune response, particularly T-cell mediated immune responses. Exemplary tumor antigens include, but not limited to, a glioma-associated antigen, carcinoembryonic antigen (CEA), β-human chorionic gonadotropin, alphafetoprotein (AFP), lectin-reactive thyroglobulin, human telomerase reverse transcriptase, RU1, RU2 (AS), intestinal carboxyl esterase, mut hsp70-2, M-CSF, prostase, prostate-specific antigen (PSA), PAP, LAGE-1a, prostein, PSMA, HER2/neu, survivin and telomerase, prostate-carcinoma tumor antigen-1 (PCTA-1), ELF2M, neutrophil elastase, ephrinB2, insulin growth factor (IGF)-I, IGF-II, IGF-I receptor, glioma-associated antigen, β-human chorionic gonadotropin, lectin-reactive AFP, thyroglobulin, RAGE-1, MN-CAIX, 707-AP, a biotinylated molecule, a-Actinin-4, abl-bcr alb-b3 (b2a2), abl-bcr alb-b4 (b3a2), adipophilin, AIM-2, Annexin II, ART-4, BAGE, BCMA, b-Catenin, bcr-abl, bcr-abl p190 (e1a2), bcr-abl p210 (b2a2), bcr-abl p210 (b3a2), BING-4, CA-125, CAG-3, CAIX, CAMEL, Caspase-8, CD171, CD19, CD20, CD22, CD23, CD24, CD30, CD33, CD38, CD44v7/8, CD70, CD123, CD133, CDC27, CDK-4, CEA, CLCA2, CLL-1, CTAGIB, Cyp-B, DAM-10, DAM-6, DEK-CAN, DLL3, EGFR, EGFRvIII, EGP-2, EGP-40, ELF2, Ep-CAM, EphA2, EphA3, erb-B2, erb-B3, erb-B4, ES-ESO-1a, ETV6/AML, FAP, FBP, fetal acetylcholine receptor, FGF-5, FN, FR-α, G250, GAGE-1, GAGE-2, GAGE-3, GAGE-4, GAGE-5, GAGE-6, GAGE-7B, GAGE-8, GD2, GD3, GnT-V, Gp100, gp75, GPC3, GPC-2, GUCY2C, Her-2, HLA-A*0201-R170I, HMW-MAA, HSP70-2 M, HST-2 (FGF6), HST-2/neu, hTERT, iCE, IL-11 Ra, IL-13Rα2, KDR, KIAA0205, K-RAS, L1-cell adhesion molecule, LAGE-1, LDLR/FUT, Lewis Y, L1-CAM, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, and K-light chain. In some embodiments, the second antigen is a receptor or ligand expressed on the cell that can interact with the T cell. In some embodiments, the second antigen is selected from a group consisting of CD40, CLL1, FLT3, FLT3L, 4-1BB, 4-1BBL, GITR, GITRL, CD27, CD70, OX40, OX40L, PD-1, PD-L1, PD-L2, Galectin-9, B7-H3, B7-H4, ICAM1, ICOS, ICOSL, CD30, CD30L, TIM1, TIM3, TIM4, SEMA4A, CD155, TIGIT, CD160, CD28, CD80, CD86, CTLA4, LAG3, LFA-1, LTβR, and HVEM.

In some embodiments, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA).

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include: differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7.

Other large, protein-based antigens include TSP-180, MAGE-4, MAGE-5, MAGE-6, RAGE, NY-ESO, pl85erbB2, pl80erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAGI6, TA-90\Mac-2 binding protein\cyclophilin C-associated protein, TAAL6, TAG72, TLP, and TPS.

Additional non-limiting exemplary targets of the CARs provided herein include GPC2, CD276, Delta-like protein ligand 3 (DLL3), NY-ESO-1, melanoma associated antigen 4; survivin protein, synovial sarcoma X breakpoint protein 2, CD3, epidermal growth factor receptor (EGFR), erbb2 tyrosine kinase receptor, HER2, CEA, CD66, CD66e, ROR1, ntrkr1 tyrosine kinase receptor, GPC3, mesothelin, glutamate carboxypeptidase II, PMSA, PD-L1, folate receptor alpha, PSCA, Mucin 1, HLA antigen (such as HLA class I antigen A-2 alpha, HLA class I antigen A-11 alpha, and HLA class II antigen), c-Met, hepatocyte growth factor receptor, K-Ras GTPase (KRAS), IL-15 receptor, Kit tyrosine kinase, PDGF receptor beta, RET tyrosine kinase receptor; Raf 1 protein kinase, Raf B protein kinase, thymidylate synthase, topoisomerase II, Brachyury protein, Flt3 tyrosine kinase, VEGF, VEGF receptor (VEGF-1 receptor, VEGF-2 receptor, and VEGF-3 receptor), estrogen receptor, neoantigen, human papillomavirus E6, and heat shock protein.

In some specific embodiments, at least one target antigen of the present CARs is CD19. In other specific embodiments, at least one target antigen of the present CARs is CD20. In yet other specific embodiments, at least one target antigen of the present CARs is CD22. In yet other specific embodiments, at least one target antigen of the present CARs is BCMA. In yet other specific embodiments, at least one target antigen of the present CARs is VEGFR2. In yet other specific embodiments, at least one target antigen of the present CARs is DLL3. In yet other specific embodiments, at least one target antigen of the present CARs is GUCY2C. In yet other specific embodiments, at least one target antigen of the present CARs is GPC2. In yet other specific embodiments, at least one target antigen of the present CARs is EpCam. In yet other specific embodiments, at least one target antigen of the present CARs is GPC3. In yet other specific embodiments, at least one target antigen of the present CARs is CD133. In yet other specific embodiments, at least one target antigen of the present CARs is IL13Ra. In yet other specific embodiments, at least one target antigen of the present CARs is EGFRIII. In yet other specific embodiments, at least one target antigen of the present CARs is EphA2. In yet other specific embodiments, at least one target antigen of the present CARs is Muc1. In yet other specific embodiments, at least one target antigen of the present CARs is CD70. In yet other specific embodiments, at least one target antigen of the present CARs is CD123. In yet other specific embodiments, at least one target antigen of the present CARs is ROR1. In yet other specific embodiments, at least one target antigen of the present CARs is PSMA. In yet other specific embodiments, at least one target antigen of the present CARs is CD5. In yet other specific embodiments, at least one target antigen of the present CARs is GD2. In yet other specific embodiments, at least one target antigen of the present CARs is GAP. In yet other specific embodiments, at least one target antigen of the present CARs is CD33. In yet other specific embodiments, at least one target antigen of the present CARs is CEA. In yet other specific embodiments, at least one target antigen of the present CARs is PSCA. In yet other specific embodiments, at least one target antigen of the present CARs is Her2. In yet other specific embodiments, at least one target antigen of the present CARs is Mesothelin.

### Transmembrane Domain

The CARs of the present disclosure comprise a transmembrane domain that can be directly or indirectly fused to the extracellular antigen binding domain. The transmembrane domain may be derived either from a natural or from a synthetic source. As used herein, a “transmembrane domain” refers to any protein structure that is thermodynamically stable in a cell membrane, preferably an eukaryotic cell membrane. Transmembrane domains compatible for use in the CARs described herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the CAR described herein is derived from a Type I single-pass membrane protein. In some embodiments, transmembrane domains from multi-pass membrane proteins may also be compatible for use in the CARs described herein. Multi-pass membrane proteins may comprise a complex (at least 2, 3, 4, 5, 6, 7 or more) alpha helices or a beta sheet structure. In some embodiments, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

Transmembrane domains for use in the CARs described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776, the relevant disclosures of which are incorporated by reference herein.

The transmembrane domain provided herein may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane domain of the CAR provided herein comprises an artificial hydrophobic sequence. For example, a triplet of phenylalanine, tryptophan and valine may be present at the C terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte and Doolittle hydropathy analysis.

In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDl 1a, CD18), ICOS (CD278), 4-1BB (CD137), GITR, CD40, BAFFR, HVEM (LIGHTR), SLAMF7, NKp80 (KLRFl), CD160, CD19, IL-2R beta, IL-2R gamma, IL-7R a, ITGA1, VLA1, CD49a, ITGA4, IA4, CD49D, ITGA6, VLA-6, CD49f, ITGAD, CDl 1d, ITGAE, CD103, ITGAL, CDl 1a, LFA-1, ITGAM, CDl 1b, ITGAX, CDl 1c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

In some specific embodiments, the transmembrane domain is derived from CD8a or mutated CD8α. In some embodiments, the transmembrane domain is a transmembrane domain of CD8α comprising the amino acid sequence of SEQ ID NO: 5. In other specific embodiments, the transmembrane domain is derived from CD28α or mutated CD280α. In some embodiments, the transmembrane domain is a transmembrane domain of CD28α comprising the amino acid sequence of SEQ ID NO: 19.

### Intracellular Signaling Domain

The intracellular signaling domain in the CARs provided herein is responsible for activation of at least one of the normal effector functions of the immune effector cell expressing the CARs. The term “effector function” refers to a specialized function of a cell. Effector function of a T cell, for example, may be cytolytic activity or helper activity including the secretion of cytokines. Thus the term “cytoplasmic signaling domain” refers to the portion of a protein which transduces the effector function signal and directs the cell to perform a specialized function. While usually the entire cytoplasmic signaling domain can be employed, in many cases it is not necessary to use the entire chain. To the extent that a truncated portion of the cytoplasmic signaling domain is used, such truncated portion may be used in place of the intact chain as long as it transduces the effector function signal. The term cytoplasmic signaling domain is thus meant to include any truncated portion of the cytoplasmic signaling domain sufficient to transduce the effector function signal.

In some embodiments, the intracellular signaling domain comprises a primary intracellular signaling domain of an immune effector cell. In some embodiments, the CAR comprises an intracellular signaling domain consisting essentially of a primary intracellular signaling domain of an immune effector cell. “Primary intracellular signaling domain” refers to cytoplasmic signaling sequence that acts in a stimulatory manner to induce immune effector functions. In some embodiments, the primary intracellular signaling domain contains a signaling motif known as immunoreceptor tyrosine-based activation motif, or ITAM. An “ITAM,” as used herein, is a conserved protein motif that is generally present in the tail portion of signaling molecules expressed in many immune cells. The motif may comprises two repeats of the amino acid sequence YxxL/I separated by 6-8 amino acids, wherein each x is independently any amino acid, producing the conserved motif YxxL/Ix(6-8)YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. Exemplary ITAM-containing primary cytoplasmic signaling sequences include those derived from CD3 zeta (CD3z), FcR gamma (FCER1G), FcR beta (Fc Epsilon Rib), CD3 gamma, CD3 delta, CD3 epsilon, CD5, CD22, CD79a, CD79b, and CD66d.

### Co-Stimulatory Signaling Domain

In some embodiments, the CAR comprises at least one co-stimulatory signaling domain. The term “co-stimulatory signaling domain,” as used herein, refers to at least a portion of a protein that mediates signal transduction within a cell to induce an immune response such as an effector function. Many immune effector cells require co-stimulation, in addition to stimulation of an antigen-specific signal, to promote cell proliferation, differentiation and survival, as well as to activate effector functions of the cell.

The co-stimulatory signaling domain of the chimeric receptor described herein can be a cytoplasmic signaling domain from a co-stimulatory protein, which transduces a signal and modulates responses mediated by immune cells, such as T cells, NK cells, macrophages, neutrophils, or eosinophils. “Co-stimulatory signaling domain” can be the cytoplasmic portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival.

In some embodiments, the intracellular signaling domain comprises a single co-stimulatory signaling domain. In some embodiments, the intracellular signaling domain comprises two or more (such as about any of 2, 3, 4, or more) co-stimulatory signaling domains. In some embodiments, the intracellular signaling domain comprises two or more of the same co-stimulatory signaling domains. In some embodiments, the intracellular signaling domain comprises two or more co-stimulatory signaling domains from different co-stimulatory proteins, such as any two or more co-stimulatory proteins described herein. In some embodiments, the intracellular signaling domain comprises a primary intracellular signaling domain (such as cytoplasmic signaling domain of CD3z) and one or more co-stimulatory signaling domains. In some embodiments, the one or more co-stimulatory signaling domains and the primary intracellular signaling domain (such as cytoplasmic signaling domain of CD3z) are fused to each other via optional peptide linkers. The primary intracellular signaling domain, and the one or more co-stimulatory signaling domains may be arranged in any suitable order. In some embodiments, the one or more co-stimulatory signaling domains are located between the transmembrane domain and the primary intracellular signaling domain (such as cytoplasmic signaling domain of CD3z). Multiple co-stimulatory signaling domains may provide additive or synergistic stimulatory effects.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the CARs described herein. The type(s) of co-stimulatory signaling domain is selected based on factors such as the type of the immune effector cells in which the effector molecules would be expressed (e.g., T cells, NK cells, macrophages, neutrophils, or eosinophils) and the desired immune effector function (e.g., ADCC effect). Examples of co-stimulatory signaling domains for use in the CARs can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF-R/TNFRSF13C, CD27/TNFRSF7, CD27 Ligand/TNFSF7, CD30/TNFRSF8, CD30 Ligand/TNFSF8, CD40/TNFRSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the one or more co-stimulatory signaling domains are selected from the group consisting of CD27, CD28, CD137, OX40, CD30, CD40, CD3, lymphocyte function-associated antigen-1 (LFA-1), CD2, CD7, LIGHT, NKG2C, B7-H3 and ligands that specially bind to CD83.

In some embodiments, the co-stimulatory signaling domains are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants. Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation.

### Signal Peptide

In certain embodiments, the CARs provided herein may comprise a signal peptide (also known as a signal sequence) at the N-terminus of the polypeptide. In general, signal peptides are peptide sequences that target a polypeptide to the desired site in a cell. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the CARs described herein will be evident to one of skill in the art. In some embodiments, the signal peptide is derived from a molecule selected from the group consisting of CD8α, GM-CSF receptor α, and IgG1 heavy chain. In a specific embodiment, the signal peptide comprises an amino acid sequence of SEQ ID NO: 3.

### Hinge Region

In some embodiments, the CARs provided herein comprise a hinge domain that is located between the extracellular antigen binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular antigen binding domain relative to the transmembrane domain of the effector molecule can be used.

Hinge domains of antibodies, such as an IgG, IgA, IgM, IgE, or IgD antibodies, are also compatible for use in the pH-dependent chimeric receptor systems described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptors described herein. In some embodiments, the hinge domain between the C-terminus of the extracellular ligand-binding domain of an Fc receptor and the N-terminus of the transmembrane domain is a peptide linker, such as a (GxS)n linker, wherein x and n, independently can be an integer between 3 and 12, including 3, 4, 5, 6, 7, 8, 9, 10, 11, 12, or more.

The hinge domain may contain about 10-100 amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge domain is a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor.

In some specific embodiments, the hinge domain is derived from CD8α. In some embodiments, the hinge domain is a portion of the hinge domain of CD8α, e.g., a fragment containing at least 15 (e.g., 20, 25, 30, 35, or 40) consecutive amino acids of the hinge domain of CD8α. In some embodiments, the hinge domain of CD8α comprises the amino acid sequence of SEQ ID NO: 4. In other embodiments, the hinge domain is derived from CD28α. In some embodiments, the hinge domain is a portion of the hinge domain of CD28α, e.g., a fragment containing at least 15 (e.g., 20, 25, 30, 35, or 40) consecutive amino acids of the hinge domain of CD28α. In some embodiments, the hinge domain of CD28α comprises the amino acid sequence of SEQ ID NO: 18.

### Exemplary CARs

Any CARs can be used in the present disclosure, including but not limited to those specific exemplary CARs exemplified in Section 6 below.

In certain embodiments, the CAR provided herein comprises amino acid sequences with certain percent identity relative to any one of the CARs exemplified in Section 6 below. In some embodiments, provided herein is a CAR comprising or consisting of an extracellular domain having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of the CARs exemplified in Section 6 below.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

In some embodiments, amino acid sequence modification(s) of the CARs described herein are contemplated. For example, it may be desirable to optimize the binding affinity and/or other biological properties of the extracellular domain, including but not limited to specificity, thermostability, expression level, effector functions, glycosylation, reduced immunogenicity, or solubility. Thus, in addition to the extracellular domain described herein, it is contemplated that variants of the domains described herein can be prepared. For example, scFv variants can be prepared by introducing appropriate nucleotide changes into the encoding DNA, and/or by synthesis of the desired antibody or polypeptide. Those skilled in the art who appreciate that amino acid changes may alter post-translational processes of the antibody.

Variations may be a substitution, deletion, or insertion of one or more codons encoding the polypeptide that results in a change in the amino acid sequence as compared with the original antibody or polypeptide. Sites of interest for substitutional mutagenesis include the CDRs and FRs.

Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the parental polypeptides.

The polypeptides generated by conservative amino acid substitutions are included in the present disclosure. In a conservative amino acid substitution, an amino acid residue is replaced with an amino acid residue having a side chain with a similar charge. As described above, families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined. Conservative (e.g., within an amino acid group with similar properties and/or side chains) substitutions may be made, so as to maintain or not significantly change the properties.

Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gin (Q); (3) acidic: Asp (D), Glu (E); and (4) basic: Lys (K), Arg (R), His(H). Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gin; (3) acidic: Asp, Glu; (4) basic: His, Lys, Arg; (5) residues that influence chain orientation: Gly, Pro; and (6) aromatic: Trp, Tyr, Phe. For example, any cysteine residue not involved in maintaining the proper conformation of the antibody also may be substituted, for example, with another amino acid, such as alanine or serine, to improve the oxidative stability of the molecule and to prevent aberrant crosslinking. Non-conservative substitutions will entail exchanging a member of one of these classes for another class.

One type of substitutional variant involves substituting one or more hypervariable region residues of a parent antibody (e.g., a humanized or human antibody) or fragment thereof in the extracellular antigen binding domain of the present CARs. Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant antibody or fragment thereof being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling.

In some embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some embodiments, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells, Science, 244:1081-1085 (1989). In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions.

Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

The variations can be made using methods known in the art such as oligonucleotide-mediated (site-directed) mutagenesis, alanine scanning, and PCR mutagenesis. Site-directed mutagenesis (see, e.g., Carter, Biochem J. 237:1-7 (1986); and Zoller et al., Nucl. Acids Res. 10:6487-500 (1982)), cassette mutagenesis (see, e.g., Wells et al., Gene 34:315-23 (1985)), or other known techniques can be performed on the cloned DNA to produce the antibody variant DNA.

5.2.2. Domains Capable of Binding to a Second Antigen

The present polypeptides include a binding domain in addition to the CAR or TCR. This additional domain is capable of binding to at least another one antigen (e.g., a receptor or ligand) on an antigen presenting cell. In some embodiments, the domain comprises two or more antigen binding domains, wherein the two or more antigen binding domains can be linked directly or via a peptide linker.

The cell that can interact with the T cell includes any cell capable of presenting the first antigen (e.g., a tumor associated antigen) to the T cell. In some embodiments, the cell that can interact with the T cell induces a response from the T cell (e.g., an APC mediated cellular immune response) upon interaction. In some embodiments, the cell that can interact with the T cell is an antigen-presenting cell (APC). The term “APC” used herein refers a broad group of cells including any cells capable of presenting an antigen to an immune cell (such as a T cell). APCs are a heterogeneous group of cells that mediate the cellular immune response by processing and presenting antigens for recognition by certain lymphocytes such as T cells. The APCs provided herein include both professional and non-professional APCs. In some embodiments, the APCs provided herein express MHC class II molecules, optionally along with co-stimulatory molecules and pattern recognition receptors. In other embodiments, the APCs provided herein express MHC class I molecules. In some embodiments, the cell that can interact with the T cell is an APC selected from a group consisting of macrophage, dendritic cell, B lymphocyte (B cell), mast cell, basophil, eosinophil, group 3 innate lymphoid cell (ILC3), monocyte, neutrophil, natural killer cell, fibroblastic reticular cell, endothelial cell, pericyte, epithelial cell, fibroblast and artificial APC cell (aAPC). In some embodiments, the cell that can interact with the T cell is an APC selected from a group consisting of macrophage, dendritic cell, and B lymphocyte (B cell). In some embodiments, the APC provided herein is a cancer cell.

In some embodiments, the second antigen is a receptor or ligand expressed on the cell that can interact with the T cell. In some embodiments, the receptor or ligand is an activating receptor or ligand. In other embodiments, the receptor or ligand is an inhibitory receptor or ligand. In yet other embodiments, the receptor or ligand is a structural receptor or ligand without any promoting or inhibitory activities.

Exemplary receptors and ligands include, but not limited to, CD40, CLL1, FLT3, FLT3L, 4-1BB, 4-1BBL, GITR, GITRL, CD27, CD70, OX40, OX40L, PD-1, PD-L1, PD-L2, Galectin-9, B7-H3, B7-H4, ICAM1, ICOS, ICOSL, CD30, CD30L, TIM1, TIM3, TIM4, SEMA4A, CD155, TIGIT, CD160, CD28, CD80, CD86, CTLA4, LAG3, LFA-1, LTβR, and HVEM.

In some embodiments, the second antigen is CD40. In some embodiments, the second antigen is CLL1. In some embodiments, the second antigen is FLT3. In some embodiments, the second antigen is FLT3L. In some embodiments, the second antigen is 4-1BB. In some embodiments, the second antigen is 4-1BBL. In some embodiments, the second antigen is GITR. In some embodiments, the second antigen is GITRL. In some embodiments, the second antigen is CD27. In some embodiments, the second antigen is CD70. In some embodiments, the second antigen is OX40. In some embodiments, the second antigen is OX40L. In some embodiments, the second antigen is PD-1. In some embodiments, the second antigen is PD-L1. In some embodiments, the second antigen is PD-L2. In some embodiments, the second antigen is Galectin-9. In some embodiments, the second antigen is B7-H3. In some embodiments, the second antigen is B7-H4. In some embodiments, the second antigen is ICAM1. In some embodiments, the second antigen is ICOS. In some embodiments, the second antigen is ICOSL. In some embodiments, the second antigen is CD30. In some embodiments, the second antigen is CD30L. In some embodiments, the second antigen is TIM1. In some embodiments, the second antigen is TIM3. In some embodiments, the second antigen is TIM4. In some embodiments, the second antigen is SEMA4A. In some embodiments, the second antigen is CD155. In some embodiments, the second antigen is TIGIT. In some embodiments, the second antigen is CD160. In some embodiments, the second antigen is CD28. In some embodiments, the second antigen is CD80. In some embodiments, the second antigen is CD86. In some embodiments, the second antigen is CTLA4. In some embodiments, the second antigen is LAG3. In some embodiments, the second antigen is LFA-1. In some embodiments, the second antigen is LTβR. In some embodiments, the second antigen is HVEM.

Other exemplary receptors and ligands applicable in the present disclosure, based on which the interation between the additional domain and the second antigen can be established, are listed in Table 2 below.

TABLE 2
Exemplary Receptors and Ligands

| Receptors | Ligands |
|---|---|
| CD40 | CD40L(CD154) |
| FLT3 | FLT3L |
| 4-1BB | 4-1BBL |
| CD27 | CD70 |
| GITR | GITRL |
| OX40 | OX40L |
| LTbR | Lymphotoxin alpha beta (abb or |
| | aaa)/LIGHT (TNFSF14) |
| HVEM | LIGHT(TNFSF14)/CD160/BTLA |
| LFA-1 | ICAM-1 |
| ICAM-1(CD54) | MAC-1(ITGB2/ITGAM)/LFA-1/Fibrinogen |
| ICOS | B7-H2(ICOSLG) |
| CD30 | CD30L (CD153) |
| SEMA4A | TIM-2/PLXNB1/PLXNB2/PLXNB3/PLXND1 |
| DNAM-1 | CD155 |
| CD96(TACTILE) | CD155 |
| TIGIT | CD155 |
| CD160 | MHC ligands(HLA-C) |
| CD28 | B7-1(CD80)/B7-2(CD86) |
| CTLA4 | B7-1(CD80)/B7-2(CD86) |
| LAG3 | MHC-II |
| PD-1 | B7-H1(PD-L1)/B7-DC(PD-L2) |
| TIM3 | Galectin-9/HMGB1 |
| TIM4 | TIM1 |
| P-selectin | TIM1/PSGL1 |
| E-selectin | CLA/CD43/CD44/DR3 |
| L-selectin | GlyCAM-1/CD34/PODXL/PSGL-1 |

In some specific embodiments, the second antigen is LTβR. In some embodiments, the domain capable of binding to the second antigen comprises a LTα or variant thereof. In other embodiments, the domain capable of binding to the second antigen comprises a LTβ or variant thereof. In yet other embodiments, the domain capable of binding to the second antigen comprises a LTα or variant thereof and a LTβ or variant thereof (also described in present patent LTα/β or mutant LTα/β).

In some embodiments, the LTα or variant thereof comprises an amino acid sequence of SEQ ID NO: 7. In some embodiments, the LTα or variant thereof comprises or consists of an amino acid sequence having at least 80%, 85%, 90%, 91%, 92%, 93%, 94%, 95%, 96%, 97%, 98%, or 99% identify to SEQ ID NO: 7. In some embodiments, the LTα or variant thereof comprises an amino acid sequence of SEQ ID NO: 8. In some embodiments, the LTα or variant thereof comprises or consists of an amino acid sequence having at least 80%, 85%, 90%, 91%, 92%, 93%, 94%, 95%, 96%, 97%, 98%, or 99% identify to SEQ ID NO: 8. In some embodiments, the LTα or variant thereof comprises an amino acid sequence of SEQ ID NO. 9. In some embodiments, the LTα or variant thereof comprises or consists of an amino acid sequence having at least 80%, 85%, 90%, 91%, 92%, 93%, 94%, 95%, 96%, 97%, 98%, or 99% identify to SEQ ID NO: 9.

In some embodiments, the LTβ or variant thereof comprises an amino acid sequence of SEQ ID NO: 10. In some embodiments, the LTβ or variant thereof comprises or consists of an amino acid sequence having at least 80%, 85%, 90%, 91%, 92%, 93%, 94%, 95%, 96%, 97%, 98%, or 99% identify to SEQ ID NO: 10. In some embodiments, the LTβ or variant thereof comprises an amino acid sequence of SEQ ID NO: 11. In some embodiments, the LTβ or variant thereof comprises or consists of an amino acid sequence having at least 80%, 85%, 90%, 91%, 92%, 93%, 94%, 95%, 96%, 97%, 98%, or 99% identify to SEQ ID NO: 11. In some embodiments, the LTβ or variant thereof comprises an amino acid sequence of SEQ ID NO: 12. In some embodiments, the LTβ or variant thereof comprises or consists of an amino acid sequence having at least 80%, 85%, 90%, 91%, 92%, 93%, 94%, 95%, 96%, 97%, 98%, or 99% identify to SEQ ID NO: 12.

In some more specific embodiments, the domain capable of binding to the second antigen comprises an amino acid sequence of SEQ ID NO: 7 and an amino acid sequence of SEQ ID NO: 10. In some more specific embodiments, the domain capable of binding to the second antigen comprises an amino acid sequence of SEQ ID NO: 7 and an amino acid sequence of SEQ ID NO: 11. In some more specific embodiments, the domain capable of binding to the second antigen comprises an amino acid sequence of SEQ ID NO: 7 and an amino acid sequence of SEQ ID NO: 12. In some more specific embodiments, the domain capable of binding to the second antigen comprises an amino acid sequence of SEQ ID NO: 8 and an amino acid sequence of SEQ ID NO: 10. In some more specific embodiments, the domain capable of binding to the second antigen comprises an amino acid sequence of SEQ ID NO: 8 and an amino acid sequence of SEQ ID NO: 11. In some more specific embodiments, the domain capable of binding to the second antigen comprises an amino acid sequence of SEQ ID NO: 8 and an amino acid sequence of SEQ ID NO: 12. In some more specific embodiments, the domain capable of binding to the second antigen comprises an amino acid sequence of SEQ ID NO: 9 and an amino acid sequence of SEQ ID NO: 10. In some more specific embodiments, the domain capable of binding to the second antigen comprises an amino acid sequence of SEQ ID NO: 9 and an amino acid sequence of SEQ ID NO: 11. In some more specific embodiments, the domain capable of binding to the second antigen comprises an amino acid sequence of SEQ ID NO: 9 and an amino acid sequence of SEQ ID NO: 12.

In some embodiments, the domain capable of binding to the second antigen comprises an amino acid sequence of SEQ ID NO: 13. In some embodiments, the domain capable of binding to the second antigen comprises or consists of an amino acid sequence having at least 80%, 85%, 90%, 91%, 92%, 93%, 94%, 95%, 96%, 97%, 98%, or 99% identify to SEQ ID NO: 13. In some embodiments, the domain capable of binding to the second antigen comprises an amino acid sequence of SEQ ID NO: 14. In some embodiments, the domain capable of binding to the second antigen comprises or consists of an amino acid sequence having at least 80%, 85%, 90%, 91%, 92%, 93%, 94%, 95%, 96%, 97%, 98%, or 99% identify to SEQ ID NO: 14. In some embodiments, the domain capable of binding to the second antigen comprises an amino acid sequence of SEQ ID NO: 15. In some embodiments, the domain capable of binding to the second antigen comprises or consists of an amino acid sequence having at least 80%, 85%, 90%, 91%, 92%, 93%, 94%, 95%, 96%, 97%, 98%, or 99% identify to SEQ ID NO: 15. In some embodiments, the domain capable of binding to the second antigen comprises an amino acid sequence of SEQ ID NO: 16. In some embodiments, the domain capable of binding to the second antigen comprises or consists of an amino acid sequence having at least 80%, 85%, 90%, 91%, 92%, 93%, 94%, 95%, 96%, 97%, 98%, or 99% identify to SEQ ID NO: 16. In some embodiments, the domain capable of binding to the second antigen comprises an amino acid sequence of SEQ ID NO: 20. In some embodiments, the domain capable of binding to the second antigen comprises or consists of an amino acid sequence having at least 80%, 85%, 90%, 91%, 92%, 93%, 94%, 95%, 96%, 97%, 98%, or 99% identify to SEQ ID NO: 20.

In case the domain capable of binding to the second antigen comprises both a LTα or variant thereof and a LTβ or variant thereof, a peptide linker can be present between a LTα or variant thereof and a LTβ or variant thereof, such as a 2A self-cleaving peptide linker. More detailed description for peptide linkers is provided below.

In other embodiments, the second antigen is HVEM. In some embodiments, the domain capable of binding to the second antigen comprises a LIGHT (TNFSF14) or variant thereof. In some embodiments, the LIGHT or variant thereof comprises an amino acid sequence of SEQ ID NO: 17. In some embodiments, the LIGHT or variant thereof comprises or consists of an amino acid sequence having at least 80%, 85%, 90%, 91%, 92%, 93%, 94%, 95%, 96%, 97%, 98%, or 99% identify to SEQ ID NO. 17.

In other embodiments, the second antigen is CD40. In some embodiments, the domain capable of binding to the second antigen comprises an antibody or fragment thereof that binds CD40. In some embodiments, the antibody or fragment thereof that binds CD40 comprises an amino acid sequence of SEQ ID NO: 21. In some embodiments, the antibody or fragment thereof that binds CD40 comprises or consists of an amino acid sequence having at least 80%, 85%, 90%, 91%, 92%, 93%, 94%, 95%, 96%, 97%, 98%, or 99% identify to SEQ ID NO: 21.

In some embodiments, the domain capable of binding to the second antigen is derived from a CD40 ligand (CD40L). In other embodiments, the domain capable of binding to the second antigen comprises an amino acid sequence of SEQ ID NO: 24. In other embodiments, the domain capable of binding to the second antigen comprises or consists of an amino acid sequence having at least 80%, 85%, 90%, 91%, 92%, 93%, 94%, 95%, 96%, 97%, 98%, or 99% identify to SEQ ID NO: 24.

In some embodiments, the second antigen is CLL1. In some embodiments, the domain capable of binding to the second antigen comprises an antibody or fragment thereof that binds CLL1. In some embodiments, the antibody or fragment thereof that binds CLL1 comprises an amino acid sequence of SEQ ID NO: 22. In some embodiments, the antibody or fragment thereof that binds CLL1 comprises or consists of an amino acid sequence having at least 80%, 85%, 90%, 91%, 92%, 93%, 94%, 95%, 96%, 97%, 98%, or 99% identify to SEQ ID NO: 22. In some embodiments, the antibody or fragment thereof that binds CLL1 comprises an amino acid sequence of SEQ ID NO: 23. In some embodiments, the antibody or fragment thereof that binds CLL1 comprises or consists of an amino acid sequence having at least 80%, 85%, 90%, 91%, 92%, 93%, 94%, 95%, 96%, 97%, 98%, or 99% identify to SEQ ID NO: 23.

In certain embodiments, the domain capable of binding a second antigen on an antigen presenting cell provided herein comprises additional amino acid sequences, such as a signal peptide, transmembrance domain, and/or a hinge region, to facilitate, for example, to locate the domain at cell membrane.

In some embodiments, the domain capable of binding a second antigen on an antigen presenting cell provided herein comprises a signal peptide (also known as a signal sequence) at the N-terminus of the domain. In some embodiments, signal peptides target the domain to the desired site in a cell. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the domains described herein will be evident to one of skill in the art.

In some embodiments, the domain capable of binding a second antigen on an antigen presenting cell provided herein comprises a transmembrane domain. The transmembrane domain may be derived either from a natural or from a synthetic source. Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain described herein is derived from a Type I single-pass membrane protein. In some embodiments, transmembrane domains from multi-pass membrane proteins may also be compatible for use in the domains described herein. Multi-pass membrane proteins may comprise a complex (at least 2, 3, 4, 5, 6, 7 or more) alpha helices or a beta sheet structure. In some embodiments, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

Transmembrane domains for use in the domains described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776, the relevant disclosures of which are incorporated by reference herein.

The transmembrane domain provided herein may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane domain comprises an artificial hydrophobic sequence. For example, a triplet of phenylalanine, tryptophan and valine may be present at the C terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte and Doolittle hydropathy analysis.

In some embodiments, the transmembrane domain comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDl 1a, CD18), ICOS (CD278), 4-1BB (CD137), GITR, CD40, BAFFR, HVEM (LIGHTR), SLAMF7, NKp80 (KLRFl), CD160, CD19, IL-2R beta, IL-2R gamma, IL-7R a, ITGA1, VLA1, CD49a, ITGA4, IA4, CD49D, ITGA6, VLA-6, CD49f, ITGAD, CDl 1d, ITGAE, CD103, ITGAL, CDl 1a, LFA-1, ITGAM, CDl 1b, ITGAX, CDl 1c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

In some specific embodiments, the transmembrane domain is derived from CD8α or mutated CD8α. In some embodiments, the transmembrane domain is a transmembrane domain of CD8α comprising the amino acid sequence of SEQ ID NO: 5. In other specific embodiments, the transmembrane domain is derived from CD28α or mutated CD28α. In some embodiments, the transmembrane domain is a transmembrane domain of CD28α comprising the amino acid sequence of SEQ ID NO: 19.

In some embodiments, the domains capable of binding a second antigen on an antigen presenting cell provided herein comprise a hinge domain. A hinge domain is an amino acid segment that is generally found between two domains of a protein and may allow for flexibility of the protein and movement of one or both of the domains relative to one another. Any amino acid sequence that provides such flexibility and movement can be used.

Hinge domains of antibodies, such as an IgG, IgA, IgM, IgE, or IgD antibodies, are also compatible for use herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

The hinge domain may contain about 10-100 amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge domain is a hinge domain of a naturally occurring protein. Hinge domains of any protein known in the art to comprise a hinge domain are compatible for use herein. Non-naturally occurring peptides may also be used as hinge domains.

In some specific embodiments, the hinge domain is derived from CD8α. In some embodiments, the hinge domain is a portion of the hinge domain of CD8α, e.g., a fragment containing at least 15 (e.g., 20, 25, 30, 35, or 40) consecutive amino acids of the hinge domain of CD8α. In some embodiments, the hinge domain of CD8α comprises the amino acid sequence of SEQ ID NO: 4. In other embodiments, the hinge domain is derived from CD28α. In some embodiments, the hinge domain is a portion of the hinge domain of CD28α, e.g., a fragment containing at least 15 (e.g., 20, 25, 30, 35, or 40) consecutive amino acids of the hinge domain of CD28α. In some embodiments, the hinge domain of CD28α comprises the amino acid sequence of SEQ ID NO: 18.

In case two or more antigen binding domains are present in the domain capable of binding a second antigen on an antigen presenting cell, a peptide linker may be used to link these antigen binding domains. The peptide linkers may be the same or different. Each peptide linker may have the same or different length and/or sequence depending on the structural and/or functional features of the various domains. Each peptide linker may be selected and optimized independently. The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a flexible linker. Exemplary flexible linkers include but not limited to glycine polymers (G)ₙ, glycine-serine polymers (including, for example, (GS)ₙ, (GSGGS)ₙ (SEQ ID NO: 28), (GGGS)ₙ (SEQ ID NO: 29), and (GGGGS)ₙ (SEQ ID NO: 30), where n is an integer of at least one), glycine-alanine polymers, alanine-serine polymers, and other flexible linkers known in the art. Exemplary peptide linkers are listed in the table below. Other linkers known in the art, for example, as described in WO2016014789, WO2015158671, WO2016102965, US20150299317, WO2018067992, U.S. Pat. No. 7,741,465, Colcher et al., J. Nat. Cancer Inst. 82:1191-1197 (1990), and Bird et al., Science 242:423-426 (1988) may also be included in the CARs provided herein, the disclosure of each of which is incorporated herein by reference. In addition, linkers cleavable in cells such as 2A self-cleaving peptides may be used. In some embodiments, the 2A self-cleaving peptide is selected from a group consisting of F2A, E2A, P2A, T2A, or variants thereof. More detailed description of such self-cleaving peptide linkers is provided in Section 5.2.3 below.

5.2.3. Peptide Linkers

Any linkers that are cleavable in cells may be used in the present disclosure to link the CARs and the domains capable of binding the second antigen expressed on an antigen presenting cell.

In some embodiments, the peptide linker is a 2A self-cleaving peptide. The members of 2A peptides are named after the virus in which they have been first described. For example, F2A, the first described 2A peptide, is derived from foot-and-mouth disease virus. The self-cleaving 18-22 amino acids long 2A peptides mediate ‘ribosomal skipping’ between the proline and glycine residues and inhibit peptide bond formation without affecting downstream translation. These peptides allow multiple proteins to be encoded as polyproteins, which dissociate into component proteins upon translation. Self-cleaving peptides are found in members of the picornaviridae virus family, including aphthoviruses such as foot-and-mouth disease virus (FMDV), equine rhinitis A virus (ERAV), Thosea asigna virus (TaV) and porcine teschovirus-1 (PTV-1) (see Donnelly et al., J. Gen. Virol., 82: 1027-101 (2001); Ryan et al., J. Gen. Virol., 72: 2727-2732 (2001)) and cardioviruses such as theilovirus (e.g., theiler's murine encephalomyelitis) and encephalomyocarditis viruses. The 2A peptides derived from FMDV, ERAV, PTV-1, and TaV are sometimes referred to as “F2A,” “E2A,” “P2A,” and “T2A,” respectively, and are included in the present disclosure, e.g., as described in Donnelly et al., J. Gen. Virol., 78: 13-21 (1997); Ryan and Drew, EMBO J., 13: 928-933 (1994); Szymczak et al., Nature Biotech., 5: 589-594 (2004); Hasegawa et al., Stem Cells, 25(7): 1707-12 (2007). In yet other embodiments, intein mediated protein splicing system is used herein, e.g., as described in Shah and Muir, Chem Sci., 5(1): 446-461 (2014) and Topilina and Mills, Mobile DNA, 5 (5) (2014). Other methods known in the art can also be used in the present constructs.

In some embodiments, the 2A self-cleaving peptide is selected from a group consisting of F2A, E2A, P2A, T2A, or variants thereof. In some embodiments, the 2A self-cleaving peptide is a P2A peptide comprising an amino acid sequence of SEQ ID NO: 2.

### 5.3. Polynucleotides

In another aspect, the disclosure provides polynucleotides that encode the polypeptide provided herein, including those described in Section 5.2 above. More specifically, provided herein is a polynucleotide encoding a polypeptide comprising (a) a chimeric antigen receptor (CAR) comprising (i) an extracellular domain capable of binding to a first antigen, (ii) a transmembrane domain, and (iii) an intracellular domain; and (b) a domain capable of binding to a second antigen expressed on the surface of a cell that can interact with a T cell, wherein the CAR and the domain are fused by a peptide linker.

In yet another aspect, provided herein is a polynucleotide comprising a first region encoding a CAR comprising (i) an extracellular domain capable of binding to a first antigen, (ii) a transmembrane domain, and (iii) an intracellular domain; and a second region encoding a domain capable of binding to a second antigen expressed on the surface of a cell that can interact with a T cell, wherein the cell that can interact with the T cell is capable of presenting the first antigen to the T cell and/or inducing a response from the T cell upon interaction. The CAR and the domain capable of binding to the second antigen are as described in Section 5.2 above. In some embodiments, the first region and the second region are controlled by the same promoter. For example, in some embodiments, internal ribosomal entry sites (IRES) are used herein to express multiple genes from one promoter. In other embodiments, the first region and the second region are controlled by separate promoters.

In yet another aspect, provided herein is a first polynucleotide encoding a CAR comprising (i) an extracellular domain capable of binding to a first antigen, (ii) a transmembrane domain, and (iii) an intracellular domain; and a second polynucleotide encoding a domain capable of binding to a second antigen expressed on the surface of a cell that can interact with a T cell, wherein the cell that can interact with the T cell is capable of presenting the first antigen to the T cell and/or inducing a response from the T cell upon interaction. The CAR and the domain capable of binding to the second antigen are as described in Section 5.2 above.

In other embodiments, provided herein is a polynucleotide encoding a polypeptide comprising (a) a TCR domain capable of binding to a first antigen, and (b) a domain capable of binding to a second antigen expressed on the surface of a cell that can interact with a T cell, wherein the TCR and the domain are fused by a peptide linker.

In yet another aspect, provided herein is a polynucleotide comprising a first region encoding a TCR domain capable of binding to a first antigen, and a second region encoding a domain capable of binding to a second antigen expressed on the surface of a cell that can interact with a T cell, wherein the cell that can interact with the T cell is capable of presenting the first antigen to the T cell and/or inducing a response from the T cell upon interaction. In some embodiments, the first region and the second region are controlled by the same promoter. For example, in some embodiments, internal ribosomal entry sites (IRES) are used herein to express multiple genes from one promoter. In other embodiments, the first region and the second region are controlled by separate promoters.

In yet another aspect, provided herein is a first polynucleotide encoding a TCR domain capable of binding to a first antigen, and a second polynucleotide encoding a domain capable of binding to a second antigen expressed on the surface of a cell that can interact with a T cell, wherein the cell that can interact with the T cell is capable of presenting the first antigen to the T cell and/or inducing a response from the T cell upon interaction.

The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide is in the form of cDNA. In some embodiments, the polynucleotide is a synthetic polynucleotide.

The present disclosure further relates to variants of the polynucleotides described herein, wherein the variant encodes, for example, fragments, analogs, and/or derivatives of the polypeptide of the disclosure. In certain embodiments, the present disclosure provides a polynucleotide comprising a polynucleotide having a nucleotide sequence at least about 75% identical, at least about 80% identical, at least about 85% identical, at least about 90% identical, at least about 95% identical, and in some embodiments, at least about 96%, 97%, 98% or 99% identical to a polynucleotide encoding the polypeptide of the disclosure. As used herein, the phrase “a polynucleotide having a nucleotide sequence at least, for example, 95% “identical” to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

### 5.4. Vectors

Also provided are vectors comprising the polynucleotides or nucleic acid molecules described herein. In one embodiment, the nucleic acid molecules can be incorporated into a recombinant expression vector.

The present disclosure provides vectors for cloning and expressing any one of the polypeptides described herein. In some embodiments, the vector is suitable for replication and integration in eukaryotic cells, such as mammalian cells. In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook el al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the engineered mammalian cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the immunomodulator (such as immune checkpoint inhibitor) coding sequence and/or self-inactivating lentiviral vectors carrying chimeric antigen receptors can be packaged with protocols known in the art. The resulting lentiviral vectors can be used to transduce a mammalian cell (such as primary human T cells) using methods known in the art. Vectors derived from retroviruses such as lentivirus are suitable tools to achieve long-term gene transfer, because they allow long-term, stable integration of a transgene and its propagation in progeny cells. Lentiviral vectors also have low immunogenicity, and can transduce non-proliferating cells.

In some embodiments, the vector comprises any one of the nucleic acids encoding a polypeptide described herein. The nucleic acid can be cloned into the vector using any known molecular cloning methods in the art, including, for example, using restriction endonuclease sites and one or more selectable markers. In some embodiments, the nucleic acid is operably linked to a promoter. Varieties of promoters have been explored for gene expression in mammalian cells, and any of the promoters known in the art may be used in the present disclosure. Promoters may be roughly categorized as constitutive promoters or regulated promoters, such as inducible promoters.

In some embodiments, the nucleic acid encoding the polypeptide is operably linked to a constitutive promoter. Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1 alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies. For example, Michael C. Milone et al compared the efficiencies of CMV, hEF1α, UbiC and PGK to drive chimeric antigen receptor expression in primary human T cells, and concluded that hEF1α promoter not only induced the highest level of transgene expression, but was also optimally maintained in the CD4 and CD8 human T cells (Molecular Therapy, 17(8): 1453-1464 (2009)). In some embodiments, the nucleic acid encoding the CAR is operably linked to a hEF1α promoter.

In some embodiments, the nucleic acid encoding the polypeptide is operably linked to an inducible promoter. Inducible promoters belong to the category of regulated promoters. The inducible promoter can be induced by one or more conditions, such as a physical condition, microenvironment of the engineered immune effector cell, or the physiological state of the engineered immune effector cell, an inducer (i.e., an inducing agent), or a combination thereof.

In some embodiments, the inducing condition does not induce the expression of endogenous genes in the engineered mammalian cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the engineered mammalian cell.

In some embodiments, the vector also contains a selectable marker gene or a reporter gene to select cells expressing the polypeptide from the population of host cells transfected through lentiviral vectors. Both selectable markers and reporter genes may be flanked by appropriate regulatory sequences to enable expression in the host cells. For example, the vector may contain transcription and translation terminators, initiation sequences, and promoters useful for regulation of the expression of the nucleic acid sequences.

### 5.5. Engineered Immune Effector Cells

In another aspect, provided herein are host cells (such as immune effector cells) comprising any one of the polypeptides, polynucleotides, or vectors described herein.

In yet another aspect, provided herein is a method for making an engineered immune effector cell (e.g., a CAR-T cell or a TCR-T cell), comprising introducing the polynucleotide or the vector provided herein (e.g., as described in Section 5.3 and Section 5.4 above) into an immune effector cell (e.g., a T cell). For example, in some embodiments, provided herein is a method for making a CAR-T cell, comprising introducing the polynucleotide encoding a polypeptide comprising (a) a chimeric antigen receptor (CAR) comprising (i) an extracellular domain capable of binding to a first antigen, (ii) a transmembrane domain, and (iii) an intracellular domain; and (b) a domain capable of binding to a second antigen expressed on the surface of a cell that can interact with a T cell, wherein the CAR and the domain are fused by a peptide linker. In other embodiments, provided herein is a method of making a CAR-T cell comprising introducing into a T cell a polynucleotide comprising a first region encoding a CAR comprising (i) an extracellular domain capable of binding to a first antigen, (ii) a transmembrane domain, and (iii) an intracellular domain; and a second region encoding a domain capable of binding to a second antigen expressed on the surface of a cell that can interact with a T cell, wherein the cell that can interact with the T cell is capable of presenting the first antigen to the T cell and/or inducing a response from the T cell upon interaction. In yet other embodiments, provided herein is a method of making a CAR-T cell comprising introducing into a T cell a first polynucleotide encoding a CAR comprising (i) an extracellular domain capable of binding to a first antigen, (ii) a transmembrane domain, and (iii) an intracellular domain; and a second polypeptide encoding a domain capable of binding to a second antigen expressed on the surface of a cell that can interact with a T cell, wherein the cell that can interact with the T cell is capable of presenting the first antigen to the T cell and/or inducing a response from the T cell upon interaction.

In yet another aspect, provided herein is an engineered immune effector cell (e.g., a CAR-T cell) produced according to the method provided herein.

5.5.1. Immune Effector Cells

“Immune effector cells” are immune cells that can perform immune effector functions. In some embodiments, the immune effector cells express at least FcγRIII and perform ADCC effector function. Examples of immune effector cells which mediate ADCC include peripheral blood mononuclear cells (PBMC), natural killer (NK) cells, monocytes, cytotoxic T cells, neutrophils, and eosinophils.

In some embodiments, the immune effector cells are T cells. In some embodiments, the T cell is an α/β T cell or γ/δ T cell. In some embodiments, the α/β T cells are CD4+/CD8−, CD4−/CD8+, CD4+/CD8+, CD4−/CD8−, or combinations thereof. In some embodiments, the T cells produce IL-2, TFN, and/or TNF upon expressing the CAR and binding to the target cells. In some embodiments, the CD8+ T cells lyse antigen-specific target cells upon expressing the CAR or TCR and binding to the target cells.

In some embodiments, the immune effector cells are NK cells. In other embodiments, the immune effector cells can be established cell lines, for example, NK-92 cells.

In some embodiments, the immune effector cells are differentiated from a stem cell, such as a hematopoietic stem cell, a pluripotent stem cell, an iPS, or an embryonic stem cell.

The engineered immune effector cells are prepared by introducing the polypeptide provided herein into the immune effector cells, such as T cells. In some embodiments, the polypeptide is introduced to the immune effector cells by transfecting any one of the isolated nucleic acids or any one of the vectors described above.

Methods of introducing vectors or isolated nucleic acids into a mammalian cell are known in the art. The vectors described can be transferred into an immune effector cell by physical, chemical, or biological methods.

Physical methods for introducing the vector into an immune effector cell include calcium phosphate precipitation, lipofection, particle bombardment, microinjection, electroporation, and the like. Methods for producing cells comprising vectors and/or exogenous nucleic acids are well-known in the art. See, e.g., Sambrook et al. (2001) Molecular Cloning: A Laboratory Manual, Cold Spring Harbor Laboratory, New York. In some embodiments, the vector is introduced into the cell by electroporation.

Biological methods for introducing the vector into an immune effector cell include the use of DNA and RNA vectors. Viral vectors have become the most widely used method for inserting genes into mammalian, e.g., human cells.

Chemical means for introducing the vector into an immune effector cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

In some embodiments, RNA molecules encoding any of the polypeptides described herein may be prepared by a conventional method (e.g., in vitro transcription) and then introduced into the immune effector cells via known methods such as mRNA electroporation. See, e.g., Rabinovich et al., Human Gene Therapy 17:1027-1035 (2006).

In some embodiments, the transduced or transfected immune effector cell is propagated ex vivo after introduction of the vector or isolated nucleic acid. In some embodiments, the transduced or transfected immune effector cell is cultured to propagate for at least about any of 1 day, 2 days, 3 days, 4 days, 5 days, 6 days, 7 days, 10 days, 12 days, or 14 days. In some embodiments, the transduced or transfected immune effector cell is further evaluated or screened to select the engineered mammalian cell.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al. FEBS Letters 479: 79-82 (2000)). Suitable expression systems are well known and may be prepared using known techniques or obtained commercially.

Other methods to confirm the presence of the nucleic acid encoding the polypeptide in the engineered immune effector cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

5.5.2. Sources of T Cells

In some embodiments, prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available in the art, may be used. In some embodiments, T cells can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. Initial activation steps in the absence of calcium may lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In a further embodiment, the time period ranges from 30 minutes to 36 hours or longer and all integer values there between. In a further embodiment, the time period is at least 1, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, in some embodiments, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4+, CD25+, CD62Lhi, GITR+, and FoxP3+. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations may result in increased cell yield, cell activation, and cell expansion. Further, use of high cell concentrations may allow more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. In some embodiments, using high concentration of cells allows more efficient selection of CD8+ T cells that normally have weaker CD28 expression.

In some embodiments, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In some embodiments, the concentration of cells used is 5×10⁶/ml. In some embodiments, the concentration used can be from about 1×10⁵/ml to 1×10⁶/ml, and any integer value in between.

In some embodiments, the cells may be incubated on a rotator for varying lengths of time at varying speeds at either 2-10° C., or at room temperature.

T cells for stimulation can also be frozen after a washing step. Without being bound by theory, the freeze and subsequent thaw step may provide a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% dextran 40 and 5% dextrose, 20% human serum albumin and 7.5% DMSO, or 31.25% plasmalyte-A, 31.25% dextrose 5%, 0.45% NaCl, 10% dextran 40 and 5% dextrose, 20% human serum albumin, and 7.5% DMSO or other suitable cell freezing media containing for example, Hespan and PlasmaLyte A. The cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some embodiments, cryopreserved cells are thawed and washed as described herein and allowed to rest for one hour at room temperature prior to activation.

Also contemplated in the present disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment, a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815 (1991); Henderson et al., Immun 73:316-321 (1991); Bierer et al., Curr. Opin. Immun. 5:763-773 (1993)). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In some embodiments, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

5.5.3. Activation and Expansion of T Cells

In some embodiments, prior to or after genetic modification of the T cells with the polypeptides described herein, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, T cells can be expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD3 antibody include UCHT1, OKT3, HIT3a (BioLegend, San Diego, US) can be used as can other methods commonly known in the art (Graves J, et al., J. Immunol. 146:2102 (1991); Li B, et al., Immunology 116:487 (2005); Rivollier A, et al., Blood 104:4029 (2004)). Examples of an anti-CD28 antibody include 9.3, B-T3, XR-CD28 (Diaclone, Besancon, France) can be used as can other methods commonly known in the art (Berg et al., Transplant Proc. 30(8):3975-3977 (1998); Haanen et al., J. Exp. Med. 190(9):13191328 (1999); Garland et al., J. Immunol Meth. 227(1-2):53-63 (1999)).

In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in certain embodiments in the present disclosure.

In some embodiments, the T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

Byway of example, cell surface proteins may be ligated by allowing paramagnetic beads to which anti-CD3 and anti-CD28 are attached (3×28 beads) to contact the T cells. In one embodiment, the cells (for example, 10⁴ to 4×10⁸ T cells) and beads (for example, anti-CD3/CD28 MACSiBead particlesa at a recommended titer of 1:100) are combined in a buffer, preferably PBS (without divalent cations such as, calcium and magnesium). Those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present disclosure. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/mL is used. In another embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations may result in increased cell yield, cell activation, and cell expansion. Further, use of high cell concentrations may allow more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In some embodiments, the mixture may be cultured for several hours (about 3 hours) to about 14 days or any hourly integer value in between. In another embodiment, the mixture may be cultured for 21 days. In one embodiment, the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

5.5.4. CAR-T Cells

In yet another aspect, provided herein is an engineered immune effector cell (e.g., a CAR-T cell) expressing (a) a CAR comprising (i) an extracellular domain capable of binding to a first antigen, (ii) a transmembrane domain, and (iii) an intracellular domain; and (b) a domain capable of binding to a second antigen expressed on the surface of a cell that can interact with a T cell (e.g., APC), wherein the cell that can interact with the T cell is capable of presenting the first antigen to the T cell and/or inducing a response from the T cell upon interaction. The CAR and the domain capable of binding to a second antigen expressed on the surface of a cell that can interact with a T cell (e.g., APC) are as described in Section 5.2 above and briefly in the following paragraphs.

In some embodiments, the CAR in the present CAR-T cells comprises an extracellular antigen binding domain; a transmembrane domain; and an intracellular signaling domain. In some embodiments, the CAR further comprises one or more additional regions/domains such as a signal peptide, hinge region, co-stimulatory signaling domain, linkers, etc., each of which can be as described in Section 5.2.1 above.

Specifically, in certain embodiments, the CARs provided herein may comprise a signal peptide at the N-terminus of the polypeptide. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the CARs described herein will be evident to one of skill in the art. In some embodiments, the signal peptide is derived from a molecule selected from the group consisting of CD8α, GM-CSF receptor α, and IgG1 heavy chain. In a specific embodiment, the signal peptide comprises an amino acid sequence of SEQ ID NO: 3.

The extracellular antigen binding domain of the CARs described herein comprises one or more antigen binding domains. In some embodiments, the extracellular antigen binding domain comprises an antibody or a fragment thereof. In a specific embodiment, the extracellular antigen binding domain of the present CARs comprise a single-chain Fv (sFv or scFv). In a specific embodiment, the extracellular antigen binding domain of the present CARs comprise a single domain antibody (e.g., a V_{H}H domain). In some embodiments, the extracellular antigen binding domain comprises humanized antibodies or fragment thereof.

In certain embodiments, the extracellular antigen binding domain comprises multiple binding domains (e.g., multiple binding domains in tandem). In some embodiments, the extracellular antigen binding domain comprises multispecific antibodies or fragments thereof. In other embodiments, the extracellular antigen binding domain comprises multivalent antibodies or fragments thereof. In case there are multiple binding domains in the extracellular antigen binding domain of the present CARs. The various domains may be fused to each other via peptide linkers. In some embodiments, the domains are directly fused to each other without any peptide linkers. The peptide linkers may be the same or different. Each peptide linker may have the same or different length and/or sequence depending on the structural and/or functional features of the various domains. Each peptide linker may be selected and optimized independently. In some embodiments, the CAR-T cell provided herein comprises multiple CARs, e.g., 2 or more different CARs.

In some embodiments, the extracellular antigen binding domain provided in the present CARs recognizes an antigen that acts as a cell surface marker on target cells associated with a special disease state. In some embodiments, the antigen is a tumor antigen. Tumors express a number of proteins that can serve as a target antigen for an immune response, particularly T cell mediated immune responses. The antigens targeted by the CAR may be antigens on a single diseased cell or antigens that are expressed on different cells that each contribute to the disease. The antigens targeted by the CAR may be directly or indirectly involved in the diseases.

Tumor antigens are proteins that are produced by tumor cells that can elicit an immune response, particularly T-cell mediated immune responses. Exemplary tumor antigens include, but not limited to, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, insulin growth factor (IGF)-I, IGF-II, IGF-I receptor, and mesothelin.

In some embodiments, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA).

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include: differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7.

Other large, protein-based antigens include TSP-180, MAGE-4, MAGE-5, MAGE-6, RAGE, NY-ESO, pl85erbB2, pl80erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

Additional non-limiting exemplary targets of the CARs provided herein include GPC2, CD276, Delta-like protein ligand 3 (DLL3), NY-ESO-1, melanoma associated antigen 4; survivin protein, synovial sarcoma X breakpoint protein 2, CD3, epidermal growth factor receptor (EGFR), erbb2 tyrosine kinase receptor, HER2, CEA, CD66, CD66e, ROR1, ntrkr1 tyrosine kinase receptor, GPC3, mesothelin, glutamate carboxypeptidase II, PMSA, PD-L1, folate receptor alpha, PSCA, Mucin 1, HLA antigen (such as HLA class I antigen A-2 alpha, HLA class I antigen A-11 alpha, and HLA class II antigen), c-Met, hepatocyte growth factor receptor, K-Ras GTPase (KRAS), IL-15 receptor, Kit tyrosine kinase, PDGF receptor beta, RET tyrosine kinase receptor; Raf 1 protein kinase, Raf B protein kinase, thymidylate synthase, topoisomerase II, Brachyury protein, Flt3 tyrosine kinase, VEGF, VEGF receptor (VEGF-1 receptor, VEGF-2 receptor, and VEGF-3 receptor), estrogen receptor, neoantigen, human papillomavirus E6, and heat shock protein.

In some specific embodiments, at least one target antigen of the present CARs is CD19. In other specific embodiments, at least one target antigen of the present CARs is CD20. In yet other specific embodiments, at least one target antigen of the present CARs is CD22. In yet other specific embodiments, at least one target antigen of the present CARs is BCMA. In yet other specific embodiments, at least one target antigen of the present CARs is VEGFR2. In yet other specific embodiments, at least one target antigen of the present CARs is DLL3. In yet other specific embodiments, at least one target antigen of the present CARs is GUCY2C. In yet other specific embodiments, at least one target antigen of the present CARs is FAP. In yet other specific embodiments, at least one target antigen of the present CARs is EpCam. In yet other specific embodiments, at least one target antigen of the present CARs is GPC3. In yet other specific embodiments, at least one target antigen of the present CARs is CD133. In yet other specific embodiments, at least one target antigen of the present CARs is IL13Ra. In yet other specific embodiments, at least one target antigen of the present CARs is GPC2. In yet other specific embodiments, at least one target antigen of the present CARs is EphA2. In yet other specific embodiments, at least one target antigen of the present CARs is Muc1. In yet other specific embodiments, at least one target antigen of the present CARs is CD70. In yet other specific embodiments, at least one target antigen of the present CARs is CD123. In yet other specific embodiments, at least one target antigen of the present CARs is ROR1. In yet other specific embodiments, at least one target antigen of the present CARs is PSMA. In yet other specific embodiments, at least one target antigen of the present CARs is CD5. In yet other specific embodiments, at least one target antigen of the present CARs is GD2. In yet other specific embodiments, at least one target antigen of the present CARs is GAP. In yet other specific embodiments, at least one target antigen of the present CARs is CD33. In yet other specific embodiments, at least one target antigen of the present CARs is CEA. In yet other specific embodiments, at least one target antigen of the present CARs is PSCA. In yet other specific embodiments, at least one target antigen of the present CARs is Her2. In yet other specific embodiments, at least one target antigen of the present CARs is Mesothelin.

In some embodiments, the CARs provided herein comprise a hinge domain that is located between the extracellular antigen binding domain and the transmembrane domain. In some embodiments, the hinge domain is a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor. In some embodiments, the hinge domain is derived from CD8α. In some embodiments, the hinge domain is a portion of the hinge domain of CD8α, e.g., a fragment containing at least 15 (e.g., 20, 25, 30, 35, or 40) consecutive amino acids of the hinge domain of CD8α. In some embodiments, the hinge domain of CD8α comprises the amino acid sequence of SEQ ID NO: 4.

The CARs of the present disclosure comprise a transmembrane domain that can be directly or indirectly fused to the extracellular antigen binding domain. The transmembrane domain may be derived either from a natural or from a synthetic source. Transmembrane domains compatible for use in the CARs described herein may be obtained from a naturally occurring protein. Alternatively, it can be a synthetic, non-naturally occurring protein segment, e.g., a hydrophobic protein segment that is thermodynamically stable in a cell membrane. In some embodiments, the transmembrane domains are derived from membrane proteins of Type I, Type II or Type III. In some embodiments, the transmembrane domain of the CAR described herein is derived from a Type I single-pass membrane protein. In some embodiments, transmembrane domains from multi-pass membrane proteins may also be compatible for use in the CARs described herein. Transmembrane domains for use in the CARs described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids.

The transmembrane domain provided herein may comprise a transmembrane region and a cytoplasmic region located at the C-terminal side of the transmembrane domain. In some embodiments, the transmembrane region of the transmembrane domain comprises hydrophobic amino acid residues.

In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDl 1a, CD18), ICOS (CD278), 4-1BB (CD137), GITR, CD40, BAFFR, HVEM (LIGHTR), SLAMF7, NKp80 (KLRFl), CD160, CD19, IL-2R beta, IL-2R gamma, IL-7R a, ITGA1, VLA1, CD49a, ITGA4, IA4, CD49D, ITGA6, VLA-6, CD49f, ITGAD, CDl 1d, ITGAE, CD103, ITGAL, CDl 1a, LFA-1, ITGAM, CDl 1b, ITGAX, CDl 1c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

In some specific embodiments, the transmembrane domain is derived from CD8α. In some embodiments, the transmembrane domain is a transmembrane domain of CD80C comprising the amino acid sequence of SEQ ID NO: 5.

The intracellular signaling domain in the CARs provided herein is responsible for activation of at least one of the normal effector functions of the immune effector cell expressing the CARs. In some embodiments, the intracellular signaling domain comprises a primary intracellular signaling domain of an immune effector cell. In some embodiments, the CAR comprises an intracellular signaling domain consisting essentially of a primary intracellular signaling domain of an immune effector cell. In some embodiments, the primary intracellular signaling domain contains a signaling motif known as immunoreceptor tyrosine-based activation motif, or ITAM. Exemplary ITAM-containing primary cytoplasmic signaling sequences include those derived from CD3z, FcR gamma (FCER1G), FcR beta (Fc Epsilon Rib), CD3 gamma, CD3 delta, CD3 epsilon, CD5, CD22, CD79a, CD79b, and CD66d.

In some embodiments, the CAR comprises at least one co-stimulatory signaling domain. The co-stimulatory signaling domain of the chimeric receptor described herein can be a cytoplasmic signaling domain from a co-stimulatory protein, which transduces a signal and modulates responses mediated by immune cells. In some embodiments, the intracellular signaling domain comprises a single co-stimulatory signaling domain. In some embodiments, the intracellular signaling domain comprises two or more (such as about any of 2, 3, 4, or more) co-stimulatory signaling domains. In some embodiments, the intracellular signaling domain comprises two or more of the same co-stimulatory signaling domains. In some embodiments, the intracellular signaling domain comprises two or more co-stimulatory signaling domains from different co-stimulatory proteins, such as any two or more co-stimulatory proteins described herein. In some embodiments, the intracellular signaling domain comprises a primary intracellular signaling domain (such as cytoplasmic signaling domain of CD3z) and one or more co-stimulatory signaling domains. In some embodiments, the one or more co-stimulatory signaling domains and the primary intracellular signaling domain (such as cytoplasmic signaling domain of CD3z) are fused to each other via optional peptide linkers. The primary intracellular signaling domain, and the one or more co-stimulatory signaling domains may be arranged in any suitable order. In some embodiments, the one or more co-stimulatory signaling domains are located between the transmembrane domain and the primary intracellular signaling domain (such as cytoplasmic signaling domain of CD3z). Multiple co-stimulatory signaling domains may provide additive or synergistic stimulatory effects.

The co-stimulatory signaling domain of any co-stimulatory molecule may be compatible for use in the CARs described herein. Examples of co-stimulatory signaling domains for use in the CARs can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the one or more co-stimulatory signaling domains are selected from the group consisting of CD27, CD28, CD137, OX40, CD30, CD40, CD3, lymphocyte function-associated antigen-1 (LFA-1), CD2, CD7, LIGHT, NKG2C, B7-H3 and ligands that specially bind to CD83.

In certain embodiments, the CAR provided herein comprises amino acid sequences with certain percent identity relative to any one of the CARs exemplified in the Section 6 below. In some embodiments, provided herein is a CAR comprising or consisting of an extracellular domain having at least 75%, 80%, 85%, 86%, 87%, 88%, 89%, 90%, 91%, 92%, 93%, 94%, 95%, 96%, 97%, 98%, 99%, or 100% sequence identity to the amino acid sequence of the CARs exemplified in the Section 6 below.

In some embodiments, the domains capable of binding to a second antigen expressed on the surface of a cell that can interact with a T cell (e.g., APC) are as described above in Section 5.2.2.

More specifically, the cell that can interact with the T cell includes any cell capable of presenting the first antigen (e.g., a cancer antigen) to the T cell. In some embodiments, the cell that can interact with the T cell induces a response from the T cell upon interaction. In some embodiments, the cell that can interact with the T cell is an antigen-presenting cell (APC). In some embodiments, the APCs provided herein express MHC class 11 molecules along with co-stimulatory molecules and pattern recognition receptors. In other embodiments, the APCs provided herein express MHC class I molecules. In some embodiments, the cell that can interact with the T cell is selected from a group consisting of macrophage, dendritic cell, B lymphocyte (B cell), mast cell, basophil, eosinophil, group 3 innate lymphoid cell (ILC3), monocyte, neutrophil, natural killer cell, fibroblastic reticular cell, endothelial cell, pericyte, epithelial cell, fibroblast and artificial APC cell (aAPC). In some embodiments, the cell that can interact with the T cell is an APC cell selected from a group consisting of macrophage, dendritic cell, and B lymphocyte (B cell).

In some embodiments, the second antigen is a receptor or ligand expressed on the cell that can interact with the T cell. In some embodiments, the receptor or ligand is an activating receptor or ligand. In other embodiments, the receptor or ligand is an inhibitory receptor or ligand. In some embodiments, the second antigen is CD40. In some embodiments, the second antigen is CLL1. In some embodiments, the second antigen is FLT3. In some embodiments, the second antigen is FLT3L. In some embodiments, the second antigen is 4-1BB. In some embodiments, the second antigen is 4-1BBL. In some embodiments, the second antigen is GITR. In some embodiments, the second antigen is GITRL. In some embodiments, the second antigen is CD27. In some embodiments, the second antigen is CD70. In some embodiments, the second antigen is OX40. In some embodiments, the second antigen is OX40L. In some embodiments, the second antigen is PD-1. In some embodiments, the second antigen is PD-L1. In some embodiments, the second antigen is PD-L2. In some embodiments, the second antigen is Galectin-9. In some embodiments, the second antigen is B7-H3. In some embodiments, the second antigen is B7-H4. In some embodiments, the second antigen is ICAM1. In some embodiments, the second antigen is ICOS. In some embodiments, the second antigen is ICOSL. In some embodiments, the second antigen is CD30. In some embodiments, the second antigen is CD30L. In some embodiments, the second antigen is TIM1. In some embodiments, the second antigen is TIM3. In some embodiments, the second antigen is TIM4. In some embodiments, the second antigen is SEMA4A. In some embodiments, the second antigen is CD155. In some embodiments, the second antigen is TIGIT. In some embodiments, the second antigen is CD160. In some embodiments, the second antigen is CD28. In some embodiments, the second antigen is CD80. In some embodiments, the second antigen is CD86. In some embodiments, the second antigen is CTLA4. In some embodiments, the second antigen is LAG3. In some embodiments, the second antigen is LFA-1. In some embodiments, the second antigen is LTβR. In some embodiments, the second antigen is HVEM. Other exemplary receptors and ligands applicable in the present disclosure are listed in Table 2 above.

In some specific embodiments, the second antigen is LTβR. In some embodiments, the domain capable of binding to the second antigen comprises a LTα or variant thereof. In other embodiments, the domain capable of binding to the second antigen comprises a LTβ or variant thereof. In yet other embodiments, the domain capable of binding to the second antigen comprises a LTα or variant thereof and a LTβ or variant thereof (also described in present patent as LTα/β or mutant LTα/β).

In some embodiments, the LTα or variant thereof comprises an amino acid sequence of SEQ ID NO: 7. In some embodiments, the LTα or variant thereof comprises or consists of an amino acid sequence having at least 80%, 85%, 90%, 91%, 92%, 93%, 94%, 95%, 96%, 97%, 98%, or 99% identify to SEQ ID NO: 7. In some embodiments, the LTα or variant thereof comprises an amino acid sequence of SEQ ID NO: 8. In some embodiments, the LTα or variant thereof comprises or consists of an amino acid sequence having at least 80%, 85%, 90%, 91%, 92%, 93%, 94%, 95%, 96%, 97%, 98%, or 99% identify to SEQ ID NO: 8. In some embodiments, the LTα or variant thereof comprises an amino acid sequence of SEQ ID NO: 9. In some embodiments, the LTα or variant thereof comprises or consists of an amino acid sequence having at least 80%, 85%, 90%, 91%, 92%, 93%, 94%, 95%, 96%, 97%, 98%, or 99% identify to SEQ ID NO: 9.

In some embodiments, the LTβ or variant thereof comprises an amino acid sequence of SEQ ID NO: 10. In some embodiments, the LTβ or variant thereof comprises or consists of an amino acid sequence having at least 80%, 85%, 90%, 91%, 92%, 93%, 94%, 95%, 96%, 97%, 98%, or 99% identify to SEQ ID NO: 10. In some embodiments, the LTβ or variant thereof comprises an amino acid sequence of SEQ ID NO: 11. In some embodiments, the LTβ or variant thereof comprises or consists of an amino acid sequence having at least 80%, 85%, 90%, 91%, 92%, 93%, 94%, 95%, 96%, 97%, 98%, or 99% identify to SEQ ID NO: 11. In some embodiments, the LTβ or variant thereof comprises an amino acid sequence of SEQ ID NO: 12. In some embodiments, the LTβ or variant thereof comprises or consists of an amino acid sequence having at least 80%, 85%, 90%, 91%, 92%, 93%, 94%, 95%, 96%, 97%, 98%, or 99% identify to SEQ ID NO: 12.

In some more specific embodiments, the domain capable of binding to the second antigen comprises an amino acid sequence of SEQ ID NO: 7 and an amino acid sequence of SEQ ID NO: 10. In some more specific embodiments, the domain capable of binding to the second antigen comprises an amino acid sequence of SEQ ID NO: 7 and an amino acid sequence of SEQ ID NO: 11. In some more specific embodiments, the domain capable of binding to the second antigen comprises an amino acid sequence of SEQ ID NO: 7 and an amino acid sequence of SEQ ID NO: 12. In some more specific embodiments, the domain capable of binding to the second antigen comprises an amino acid sequence of SEQ ID NO: 8 and an amino acid sequence of SEQ ID NO: 10. In some more specific embodiments, the domain capable of binding to the second antigen comprises an amino acid sequence of SEQ ID NO: 8 and an amino acid sequence of SEQ ID NO: 11. In some more specific embodiments, the domain capable of binding to the second antigen comprises an amino acid sequence of SEQ ID NO: 8 and an amino acid sequence of SEQ ID NO: 12. In some more specific embodiments, the domain capable of binding to the second antigen comprises an amino acid sequence of SEQ ID NO: 9 and an amino acid sequence of SEQ ID NO: 10. In some more specific embodiments, the domain capable of binding to the second antigen comprises an amino acid sequence of SEQ ID NO: 9 and an amino acid sequence of SEQ ID NO: 11. In some more specific embodiments, the domain capable of binding to the second antigen comprises an amino acid sequence of SEQ ID NO: 9 and an amino acid sequence of SEQ ID NO: 12.

In some embodiments, the domain capable of binding to the second antigen comprises an amino acid sequence of SEQ ID NO: 13. In some embodiments, the domain capable of binding to the second antigen comprises or consists of an amino acid sequence having at least 80%, 85%, 90%, 91%, 92%, 93%, 94%, 95%, 96%, 97%, 98%, or 99% identify to SEQ ID NO: 13. In some embodiments, the domain capable of binding to the second antigen comprises an amino acid sequence of SEQ ID NO: 14. In some embodiments, the domain capable of binding to the second antigen comprises or consists of an amino acid sequence having at least 80%, 85%, 90%, 91%, 92%, 93%, 94%, 95%, 96%, 97%, 98%, or 99% identify to SEQ ID NO: 14. In some embodiments, the domain capable of binding to the second antigen comprises an amino acid sequence of SEQ ID NO: 15. In some embodiments, the domain capable of binding to the second antigen comprises or consists of an amino acid sequence having at least 80%, 85%, 90%, 91%, 92%, 93%, 94%, 95%, 96%, 97%, 98%, or 99% identify to SEQ ID NO: 15. In some embodiments, the domain capable of binding to the second antigen comprises an amino acid sequence of SEQ ID NO: 16. In some embodiments, the domain capable of binding to the second antigen comprises or consists of an amino acid sequence having at least 80%, 85%, 90%, 91%, 92%, 93%, 94%, 95%, 96%, 97%, 98%, or 99% identify to SEQ ID NO: 16. In some embodiments, the domain capable of binding to the second antigen comprises an amino acid sequence of SEQ ID NO: 20. In some embodiments, the domain capable of binding to the second antigen comprises or consists of an amino acid sequence having at least 80%, 85%, 90%, 91%, 92%, 93%, 94%, 95%, 96%, 97%, 98%, or 99% identify to SEQ ID NO: 20.

In other embodiments, the second antigen is HVEM. In some embodiments, the domain capable of binding to the second antigen comprises a LIGHT (TNFSF14) or variant thereof. In some embodiments, the LIGHT or variant thereof comprises an amino acid sequence of SEQ ID NO: 17. In some embodiments, the LIGHT or variant thereof comprises or consists of an amino acid sequence having at least 80%, 85%, 90%, 91%, 92%, 93%, 94%, 95%, 96%, 97%, 98%, or 99% identify to SEQ ID NO: 17.

In other embodiments, the second antigen is CD40. In some embodiments, the domain capable of binding to the second antigen comprises an antibody or fragment thereof that binds CD40. In some embodiments, the antibody or fragment thereof that binds CD40 comprises an amino acid sequence of SEQ ID NO: 21. In some embodiments, the antibody or fragment thereof that binds CD40 comprises or consists of an amino acid sequence having at least 80%, 85%, 90%, 91%, 92%, 93%, 94%, 95%, 96%, 97%, 98%, or 99% identify to SEQ ID NO: 21.

In other embodiments, the domain capable of binding to the second antigen is derived from a CD40 ligand (CD40L). In other embodiments, the domain capable of binding to the second antigen comprises an amino acid sequence of SEQ ID NO: 24. In other embodiments, the domain capable of binding to the second antigen comprises or consists of an amino acid sequence having at least 80%, 85%, 90%, 91%, 92%, 93%, 94%, 95%, 96%, 97%, 98%, or 99% identify to SEQ ID NO: 24.

In some embodiments, the second antigen is CLL1. In some embodiments, the domain capable of binding to the second antigen comprises an antibody or fragment thereof that binds CLL1. In some embodiments, the antibody or fragment thereof that binds CLL1 comprises an amino acid sequence of SEQ ID NO: 22. In some embodiments, the antibody or fragment thereof that binds CLL1 comprises or consists of an amino acid sequence having at least 80%, 85%, 90%, 91%, 92%, 93%, 94%, 95%, 96%, 97%, 98%, or 99% identify to SEQ ID NO: 22. In some embodiments, the antibody or fragment thereof that binds CLL1 comprises an amino acid sequence of SEQ ID NO: 23. In some embodiments, the antibody or fragment thereof that binds CLL1 comprises or consists of an amino acid sequence having at least 80%, 85%, 90%, 91%, 92%, 93%, 94%, 95%, 96%, 97%, 98%, or 99% identify to SEQ ID NO: 23.

In certain embodiments, the domain capable of binding a second antigen on an antigen presenting cell provided herein comprises additional amino acid sequences, such as a signal peptide, transmembrance domain, and/or a hinge region.

In some embodiments, the domain capable of binding a second antigen on an antigen presenting cell provided herein comprises a signal peptide (also known as a signal sequence) at the N-terminus of the domain. In some embodiments, signal peptides target the domain to the desired site in a cell. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the domains described herein will be evident to one of skill in the art.

In some embodiments, the domain capable of binding a second antigen on an antigen presenting cell provided herein comprises a transmembrane domain. The transmembrane domain may be derived either from a natural or from a synthetic source. Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type 1, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain described herein is derived from a Type I single-pass membrane protein. In some embodiments, transmembrane domains from multi-pass membrane proteins may also be compatible for use in the domains described herein. Multi-pass membrane proteins may comprise a complex (at least 2, 3, 4, 5, 6, 7 or more) alpha helices or a beta sheet structure. In some embodiments, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

Transmembrane domains for use in the domains described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776, the relevant disclosures of which are incorporated by reference herein.

The transmembrane domain provided herein may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane domain comprises an artificial hydrophobic sequence. For example, a triplet of phenylalanine, tryptophan and valine may be present at the C terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte and Doolittle hydropathy analysis.

In some embodiments, the transmembrane domain comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDl 1a, CD18), ICOS (CD278), 4-1BB (CD137), GITR, CD40, BAFFR, HVEM (LIGHTR), SLAMF7, NKp80 (KLRFl), CD160, CD19, IL-2R beta, IL-2R gamma, IL-7R a, ITGA1, VLA1, CD49a, ITGA4, IA4, CD49D, ITGA6, VLA-6, CD49f, ITGAD, CDl 1d, ITGAE, CD103, ITGAL, CDl 1a, LFA-1, ITGAM, CDl 1b, ITGAX, CDl 1c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

In some specific embodiments, the transmembrane domain is derived from CD8α. In some embodiments, the transmembrane domain is a transmembrane domain of CD8α comprising the amino acid sequence of SEQ ID NO: 5. In other specific embodiments, the transmembrane domain is derived from CD28α. In some embodiments, the transmembrane domain is a transmembrane domain of CD28α comprising the amino acid sequence of SEQ ID NO: 19.

In some embodiments, the domains capable of binding a second antigen on an antigen presenting cell provided herein comprise a hinge domain. A hinge domain is an amino acid segment that is generally found between two domains of a protein and may allow for flexibility of the protein and movement of one or both of the domains relative to one another. Any amino acid sequence that provides such flexibility and movement can be used.

Hinge domains of antibodies, such as an IgG, IgA, IgM, IgE, or IgD antibodies, are also compatible for use herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

The hinge domain may contain about 10-100 amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge domain is a hinge domain of a naturally occurring protein. Hinge domains of any protein known in the art to comprise a hinge domain are compatible for use herein. Non-naturally occurring peptides may also be used as hinge domains.

In some specific embodiments, the hinge domain is derived from CD8α. In some embodiments, the hinge domain is a portion of the hinge domain of CD8α, e.g., a fragment containing at least 15 (e.g., 20, 25, 30, 35, or 40) consecutive amino acids of the hinge domain of CD8α. In some embodiments, the hinge domain of CD8α comprises the amino acid sequence of SEQ ID NO: 4. In other embodiments, the hinge domain is derived from CD28α. In some embodiments, the hinge domain is a portion of the hinge domain of CD28α, e.g., a fragment containing at least 15 (e.g., 20, 25, 30, 35, or 40) consecutive amino acids of the hinge domain of CD28α. In some embodiments, the hinge domain of CD28α comprises the amino acid sequence of SEQ ID NO: 18.

In case two or more antigen binding domains are present in the domain capable of binding a second antigen on an antigen presenting cell, a peptide linker may be used to link these antigen binding domains. The peptide linkers may be the same or different. Each peptide linker may have the same or different length and/or sequence depending on the structural and/or functional features of the various domains. Each peptide linker may be selected and optimized independently. The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a flexible linker. Exemplary flexible linkers include but not limited to glycine polymers (G)ₙ, glycine-serine polymers (including, for example, (GS)ₙ, (GSGGS)ₙ, (GGGS)ₙ, and (GGGGS)ₙ, where n is an integer of at least one), glycine-alanine polymers, alanine-serine polymers, and other flexible linkers known in the art. Exemplary peptide linkers are listed in the table below. Other linkers known in the art, for example, as described in WO2016014789, WO2015158671, WO2016102965, US20150299317, WO2018067992, U.S. Pat. No. 7,741,465, Colcher et al., J. Nat. Cancer Inst. 82:1191-1197 (1990), and Bird et al., Science 242:423-426 (1988) may also be included in the CARs provided herein, the disclosure of each of which is incorporated herein by reference. In addition, linkers cleavable in cells such as 2A self-cleaving peptides may be used. In some embodiments, the 2A self-cleaving peptide is selected from a group consisting of F2A, E2A, P2A, T2A, or variants thereof. More detailed description of such self-cleaving peptide linkers is provided in Section 5.2.3 above.

In yet another aspect, provided herein is an engineered immune effector cell (e.g., a TCR-T cell) expressing (a) a TCR capable of binding to a first antigen, and (b) a domain capable of binding to a second antigen expressed on the surface of a cell that can interact with a T cell (e.g., APC), wherein the cell that can interact with the T cell is capable of presenting the first antigen to the T cell and/or inducing a response from the T cell upon interaction. The domain capable of binding to a second antigen expressed on the surface of a cell that can interact with a T cell (e.g., APC) are as described in Section 5.2 above.

### 5.6. Pharmaceutical Compositions

In one aspect, the present disclosure further provides pharmaceutical compositions comprising an engineered T cell of the present disclosure. In some embodiments, a pharmaceutical composition comprises a therapeutically effective amount of the engineered T cell of the present disclosure and a pharmaceutically acceptable excipient.

In a specific embodiment, the term “excipient” can also refer to a diluent, adjuvant (e.g., Freunds' adjuvant (complete or incomplete), carrier or vehicle. Pharmaceutical excipients can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid excipients. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical excipients are described in Remington's Pharmaceutical Sciences (1990) Mack Publishing Co., Easton, PA. Such compositions will contain a prophylactically or therapeutically effective amount of the active ingredient provided herein, such as in purified form, together with a suitable amount of excipient so as to provide the form for proper administration to the patient. The formulation should suit the mode of administration.

In some embodiments, the choice of excipient is determined in part by the particular cell, and/or by the method of administration. Accordingly, there are a variety of suitable formulations.

Typically, acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers may be used to control the pH in a range which optimizes the therapeutic effectiveness, especially if stability is pH dependent. Suitable buffering agents for use with the present disclosure include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives may be added to retard microbial growth. Suitable preservatives for use with the present disclosure include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Exemplary tonicity agents include polyhydric sugar alcohols, trihydric or higher sugar alcohols, such as glycerin, erythritol, arabitol, xylitol, sorbitol and mannitol.

Additional exemplary excipients include: (1) bulking agents, (2) solubility enhancers, (3) stabilizers and (4) agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) may be present to help solubilize the therapeutic agent as well as to protect the therapeutic protein against agitation-induced aggregation, which also permits the formulation to be exposed to shear surface stress without causing denaturation of the active therapeutic protein or antibody. Suitable non-ionic surfactants include, e.g., polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they are preferably sterile. The pharmaceutical composition may be rendered sterile by filtration through sterile filtration membranes. The pharmaceutical compositions herein generally can be placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

In another embodiment, a pharmaceutical composition can be provided as a controlled release or sustained release system. In one embodiment, a pump may be used to achieve controlled or sustained release (see, e.g., Sefton, Crit. Ref. Biomed. Eng. 14:201-40 (1987); Buchwald et al., Surgery 88:507-16 (1980); and Saudek et al., N. Engl. J. Med. 321:569-74 (1989)). In another embodiment, polymeric materials can be used to achieve controlled or sustained release of a prophylactic or therapeutic agent (e.g., a fusion protein as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61-126 (1983); Levy et al., Science 228:190-92 (1985); During et al., Ann. Neurol. 25:351-56 (1989); Howard et al., J. Neurosurg. 71.105-12 (1989); U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, Science 249:1527-33 (1990). Any technique known to one of skill in the art can be used to produce sustained release formulations comprising one or more agents as described herein (see, e.g., U.S. Pat. No. 4,526,938, PCT publication Nos. WO 91/05548 and WO 96/20698, Ning et al., Radiotherapy & Oncology 39:179-89 (1996); Song et al., PDA J. of Pharma. Sci. & Tech. 50:372-97 (1995); Cleek et al., Pro. Int'l. Symp. Control. Rel. Bioact. Mater. 24:853-54 (1997); and Lam et al., Proc. Int'l. Symp. Control Rel. Bioact. Mater. 24:759-60 (1997)).

The pharmaceutical compositions described herein may also contain more than one active compound or agent as necessary for the particular indication being treated. Alternatively, or in addition, the composition may comprise a cytotoxic agent, chemotherapeutic agent, cytokine, immunosuppressive agent, or growth inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

Various compositions and delivery systems are known and can be used with the therapeutic agents provided herein, including, but not limited to, encapsulation in liposomes, microparticles, microcapsules, recombinant cells capable of expressing the single domain antibody or therapeutic molecule provided herein, construction of a nucleic acid as part of a retroviral or other vector, etc.

In some embodiments, the pharmaceutical composition provided herein contains the binding molecules and/or cells in amounts effective to treat or prevent the disease or disorder, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined.

### 5.7. Methods and Uses

In another aspect, provided herein are methods for using and uses of the engineered T cells provided herein. Such methods and uses include therapeutic methods and uses, for example, involving administration of the cells, or compositions containing the same, to a subject having a disease or disorder. In some embodiments, the cell is administered in an effective amount to effect treatment of the disease or disorder. Uses include uses of the cells in such methods and treatments, and in the preparation of a medicament in order to carry out such therapeutic methods. In some embodiments, the methods are carried out by administering the cells, or compositions comprising the same, to the subject having or suspected of having the disease or condition. In some embodiments, the methods thereby treat the disease or disorder in the subject.

In some embodiments, the treatment provided herein cause complete or partial amelioration or reduction of a disease or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms include, but do not imply, complete curing of a disease or complete elimination of any symptom or effect(s) on all symptoms or outcomes.

As used herein, in some embodiments, the treatment provided herein delay development of a disease or disorder, e.g., defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease or disorder. For example, a late stage cancer, such as development of metastasis, may be delayed. In other embodiments, the method or the use provided herein prevents a disease or disorder.

In some embodiments, the present CAR-T cell therapies are used for treating solid tumor cancer. In other embodiments, the present CAR-T cell therapies are used for treating blood cancer. In other embodiments, the disease or disorder is an autoimmune and inflammatory disease.

In some embodiments, the disease or disorder is a disease of abnormal cell growth and/or dysregulated apoptosis. Examples of such diseases include, but are not limited to, cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In some embodiments, the disease or disorder is selected from the group consisting of bladder cancer, brain cancer, breast cancer, bone marrow cancer, cervical cancer, chronic lymphocytic leukemia, acute lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small-cell lung cancer, prostate cancer, small-cell lung cancer and spleen cancer.

In some embodiments, the disease or disorder is a hematological cancer, such as leukemia, lymphoma, or myeloma. In some embodiments, the cancer is selected from a group consisting of Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma, chronic myelogenous leukemia (CML), and chronic monocytic leukemia. In a specific embodiment, the disease or disorder is myelodysplastic syndromes (MDS). In another specific embodiment, the disease or disorder is acute myeloid leukemia (AML). In another specific embodiment, the disease or disorder is chronic lymphocytic leukemia (CLL). In yet another specific embodiment, the disease or disorder is multiple myeloma (MM).

In other embodiments, the disease or disorder is a solid tumor cancer. In some embodiments, the solid tumor cancer is selected from a group consisting of a carcinoma, an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma, a non-melanoma skin carcinoma, and a lung cancer.

In other embodiments, the disease or disorder is an immune or autoimmune disorder. Such disorders include autoimmune bullous disease, abetalipoprotemia, acquired immunodeficiency-related diseases, acute immune disease associated with organ transplantation, acquired acrocyanosis, acute and chronic parasitic or infectious processes, acute pancreatitis, acute renal failure, acute rheumatic fever, acute transverse myelitis, adenocarcinomas, aerial ectopic beats, adult (acute) respiratory distress syndrome, AIDS dementia complex, alcoholic cirrhosis, alcohol-induced liver injury, alcohol-induced hepatitis, allergic conjunctivitis, allergic contact dermatitis, allergic rhinitis, allergy and asthma, allograft rejection, alpha-1-antitrypsin deficiency, Alzheimer's disease, amyotrophic lateral sclerosis, anemia, angina pectoris, ankylosing spondylitis-associated lung disease, anterior horn cell degeneration, antibody mediated cytotoxicity, antiphospholipid syndrome, anti-receptor hypersensitivity reactions, aortic and peripheral aneurysms, aortic dissection, arterial hypertension, arteriosclerosis, arteriovenous fistula, arthropathy, asthenia, asthma, ataxia, atopic allergy, atrial fibrillation (sustained or paroxysmal), atrial flutter, atrioventricular block, atrophic autoimmune hypothyroidism, autoimmune haemo lytic anaemia, autoimmune hepatitis, type-1 autoimmune hepatitis (classical autoimmune or lupoid hepatitis), autoimmune mediated hypoglycemia, autoimmune neutropenia, autoimmune thrombocytopenia, autoimmune thyroid disease, B-cell lymphoma, bone graft rejection, bone marrow transplant (BMT) rejection, bronchiolitis obliterans, bundle branch block, burns, cachexia, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy-associated disorders, chlamydia, choleosatatis, chronic alcoholism, chronic active hepatitis, chronic fatigue syndrome, chronic immune disease associated with organ transplantation, chronic eosinophilic pneumonia, chronic inflammatory pathologies, chronic mucocutaneous candidiasis, chronic obstructive pulmonary disease (COPD), chronic salicylate intoxication, colorectal common varied immunodeficiency (common variable hypogammaglobulinemia), conjunctivitis, connective tissue disease-associated interstitial lung disease, contact dermatitis, Coombs-positive hemolytic anemia, cor pulmonale, Creutzfeldt-Jakob disease, cryptogenic autoimmune hepatitis, cryptogenic fibrosing alveolitis, culture-negative sepsis, cystic fibrosis, cytokine therapy-associated disorders, Crohn's disease, dementia pugilistica, demyelinating diseases, dengue hemorrhagic fever, dermatitis, dermatitis scleroderma, dermatologic conditions, dermatomyositis/polymyositis-associated lung disease, diabetes, diabetic arteriosclerotic disease, diabetes mellitus, diffuse Lewy body disease, dilated cardiomyopathy, dilated congestive cardiomyopathy, discoid lupus erythematosus, disorders of the basal ganglia, disseminated intravascular coagulation, Down's Syndrome in middle age, drug-induced interstitial lung disease, drug-induced hepatitis, drug-induced movement disorders induced by drugs which block CNS dopamine receptors, drug sensitivity, eczema, encephalomyelitis, endocarditis, endocrinopathy, enteropathic synovitis, epiglottitis, Epstein-Barr virus infection, erythromelalgia, extrapyramidal and cerebellar disorders, familial hematophagocytic lymphohistiocytosis, fetal thymus implant rejection, Friedreich's ataxia, functional peripheral arterial disorders, female infertility, fibrosis, fibrotic lung disease, fungal sepsis, gas gangrene, gastric ulcer, giant cell arteritis, glomerular nephritis, glomerulonephritides, Goodpasture's syndrome, goitrous autoimmune hypothyroidism (Hashimoto's disease), gouty arthritis, graft rejection of any organ or tissue, graft versus host disease, gram-negative sepsis, gram-positive sepsis, granulomas due to intracellular organisms, group B streptococci (GBS) infection, Graves' disease, hemosiderosis-associated lung disease, hairy cell leukemia, Hallerrorden-Spatz disease, Hashimoto's thyroiditis, hay fever, heart transplant rejection, hemachromatosis, hematopoietic malignancies (leukemia and lymphoma), hemolytic anemia, hemolytic uremic syndrome/thrombolytic thrombocytopenic purpura, hemorrhage, Henoch-Schoenlein purpura, hepatitis A, hepatitis B, hepatitis C, HIV infection/HIV neuropathy, Hodgkin's disease, hypoparathyroidism, Huntington's chorea, hyperkinetic movement disorders, hypersensitivity reactions, hypersensitivity pneumonitis, hyperthyroidism, hypokinetic movement disorders, hypothalamic-pituitary-adrenal axis evaluation, idiopathic Addison's disease, idiopathic leucopenia, idiopathic pulmonary fibrosis, idiopathic thrombocytopenia, idiosyncratic liver disease, infantile spinal muscular atrophy, infectious diseases, inflammation of the aorta, inflammatory bowel disease, insulin dependent diabetes mellitus, interstitial pneumonitis, iridocyclitis/uveitis/optic neuritis, ischemia-reperfusion injury, ischemic stroke, juvenile pernicious anemia, juvenile rheumatoid arthritis, juvenile spinal muscular atrophy, Kaposi's sarcoma, Kawasaki's disease, kidney transplant rejection, legionella, leishmaniasis, leprosy, lesions of the corticospinal system, linear IgA disease, lipidema, liver transplant rejection, Lyme disease, lymphederma, lymphocytic infiltrative lung disease, malaria, male infertility idiopathic or NOS, malignant histiocytosis, malignant melanoma, meningitis, meningococcemia, microscopic vasculitis of the kidneys, migraine headache, mitochondrial multisystem disorder, mixed connective tissue disease, mixed connective tissue disease-associated lung disease, monoclonal gammopathy, multiple myeloma, multiple systems degenerations (Mencel, Dejerine-Thomas, Shy-Drager and Machado-Joseph), myalgic encephalitis/Royal Free Disease, myasthenia gravis, microscopic vasculitis of the kidneys, Mycobacterium avium intracellulare, Mycobacterium tuberculosis, myelodyplastic syndrome, myocardial infarction, myocardial ischemic disorders, nasopharyngeal carcinoma, neonatal chronic lung disease, nephritis, nephrosis, nephrotic syndrome, neurodegenerative diseases, neurogenic I muscular atrophies, neutropenic fever, non-alcoholic steatohepatitis, occlusion of the abdominal aorta and its branches, occlusive arterial disorders, organ transplant rejection, orchitis/epidydimitis, orchitis/vasectomy reversal procedures, organomegaly, osteoarthrosis, osteoporosis, ovarian failure, pancreas transplant rejection, parasitic diseases, parathyroid transplant rejection, Parkinson's disease, pelvic inflammatory disease, pemphigus vulgaris, Pemphigus foliaceus, pemphigoid, perennial rhinitis, pericardial disease, peripheral atherlosclerotic disease, peripheral vascular disorders, peritonitis, pernicious anemia, phacogenic uveitis, Pneumocystis carinii pneumonia, pneumonia, POEMS syndrome (polyneuropathy, organomegaly, endocrinopathy, monoclonal gammopathy, and skin changes syndrome), post-perfusion syndrome, post-pump syndrome, post-MI cardiotomy syndrome, postinfectious interstitial lung disease, premature ovarian failure, primary biliary cirrhosis, primary sclerosing hepatitis, primary myxoedema, primary pulmonary hypertension, primary sclerosing cholangitis, primary vasculitis, progressive supranuclear palsy, psoriasis, psoriasis type 1, psoriasis type 2, psoriatic arthropathy, pulmonary hypertension secondary to connective tissue disease, pulmonary manifestation of polyarteritis nodosa, post-inflammatory interstitial lung disease, radiation fibrosis, radiation therapy, Raynaud's phenomenon and disease, Raynoud's disease, Refsum's disease, regular narrow QRS tachycardia, Reiter's disease, renal disease NOS, renovascular hypertension, reperfusion injury, restrictive cardiomyopathy, rheumatoid arthritis-associated interstitial lung disease, rheumatoid spondylitis, sarcoidosis, Schmidt's syndrome, scleroderma, senile chorea, senile dementia of Lewy body type, sepsis syndrome, septic shock, seronegative arthropathies, shock, sickle cell anemia, T-cell or FAB ALL, Takayasu's disease/arteritis, telangiectasia, Th2-type and Th1-type mediated diseases, thromboangitis obliterans, thrombocytopenia, thyroiditis, toxicity, toxic shock syndrome, transplants, trauma/hemorrhage, type-2 autoimmune hepatitis (anti-LKM antibody hepatitis), type B insulin resistance with acanthosis nigricans, type III hypersensitivity reactions, type IV hypersensitivity, ulcerative colitic arthropathy, ulcerative colitis, unstable angina, uremia, urosepsis, urticaria, uveitis, valvular heart diseases, varicose veins, vasculitis, vasculitic diffuse lung disease, venous diseases, venous thrombosis, ventricular fibrillation, vitiligo acute liver disease, viral and fungal infections, vital encephalitis/aseptic meningitis, vital-associated hemaphagocytic syndrome, Wegener's granulomatosis, Wernicke-Korsakoff syndrome, Wilson's disease, xenograft rejection of any organ or tissue, yersinia and salmonella-associated arthropathy, acquired immunodeficiency disease syndrome (AIDS), autoimmune lymphoproliferative syndrome, hemolytic anemia, inflammatory diseases, thrombocytopenia, acute and chronic immune diseases associated with organ transplantation, Addison's disease, allergic diseases, alopecia, alopecia areata, atheromatous disease/arteriosclerosis, atherosclerosis, arthritis (including osteoarthritis, juvenile chronic arthritis, septic arthritis, Lyme arthritis, psoriatic arthritis and reactive arthritis), Sjogren's disease-associated lung disease, Sjogren's syndrome, skin allograft rejection, skin changes syndrome, small bowel transplant rejection, sperm autoimmunity, multiple sclerosis (all subtypes), spinal ataxia, spinocerebellar degenerations, spondyloarthropathy, sporadic polyglandular deficiency type I, sporadic polyglandular deficiency type II, Still's disease, streptococcal myositis, stroke, structural lesions of the cerebellum, subacute sclerosing panencephalitis, sympathetic ophthalmia, syncope, syphilis of the cardiovascular system, systemic anaphylaxis, systemic inflammatory response syndrome, systemic onset juvenile rheumatoid arthritis, systemic lupus erythematosus, systemic lupus erythematosus-associated lung disease, lupus nephritis, systemic sclerosis, and systemic sclerosis-associated interstitial lung disease.

In some embodiments, the disease or disorder is an inflammatory disease. Inflammation plays a fundamental role in host defenses and the progression of immune-mediated diseases. The inflammatory response is initiated in response to injury (e.g., trauma, ischemia, and foreign particles) and infection (e.g., bacterial or viral infection) by a complex cascade of events, including chemical mediators (e.g., cytokines and prostaglandins) and inflammatory cells (e.g., leukocytes). The inflammatory response is characterized by increased blood flow, increased capillary permeability, and the influx of phagocytic cells. These events result in swelling, redness, warmth (altered heat patterns), and pus formation at the site of injury or infection.

Cytokines and prostaglandins control the inflammatory response, and are released in an ordered and self-limiting cascade into the blood or affected tissues. This release of cytokines and prostaglandins increases the blood flow to the area of injury or infection, and may result in redness and warmth. Some of these chemicals cause a leak of fluid into the tissues, resulting in swelling. This protective process may stimulate nerves and cause pain. These changes, when occurring for a limited period in the relevant area, work to the benefit of the body.

A delicate well-balanced interplay between the humoral and cellular immune elements in the inflammatory response enables the elimination of harmful agents and the initiation of the repair of damaged tissue. When this delicately balanced interplay is disrupted, the inflammatory response may result in considerable damage to normal tissue and may be more harmful than the original insult that initiated the reaction. In these cases of uncontrolled inflammatory responses, clinical intervention is needed to prevent tissue damage and organ dysfunction. Diseases such as psoriasis, rheumatoid arthritis, osteoarthritis, psoriatic arthritis, Crohn's disease, asthma, allergies or inflammatory bowel disease, are characterized by chronic inflammation. Inflammatory diseases such as arthritis, related arthritic conditions (e.g., osteoarthritis, rheumatoid arthritis, and psoriatic arthritis), inflammatory bowel disease (e.g., Crohn's disease and ulcerative colitis), sepsis, psoriasis, atopic dermatitis, contact dermatitis, and chronic obstructive pulmonary disease, chronic inflammatory pulmonary diseases are also prevalent and problematic ailments.

In some embodiments, the methods include adoptive cell therapy, whereby genetically engineered cells are administered to a subject. Such administration can promote activation of the cells (e.g., T cell activation), such that the cells of the disease or disorder are targeted for destruction.

In some embodiments, the methods include administration of the cells or a composition containing the cells to a subject, tissue, or cell, such as one having, at risk for, or suspected of having the disease or disorder. In some embodiments, the cells, populations, and compositions are administered to a subject having the particular disease or disorder to be treated, e.g., via adoptive cell therapy, such as adoptive T cell therapy. In some embodiments, the cells or compositions are administered to the subject, such as a subject having or at risk for the disease or disorder. In some embodiments, the methods thereby treat, e.g., ameliorate one or more symptom of the disease or disorder.

Methods for administration of cells for adoptive cell therapy are known, as described, e.g., in US Patent Application Publication No. 2003/0170238; U.S. Pat. No. 4,690,915; Rosenberg, Nat Rev Clin Oncol. 8 (10):577-85 (2011); Themeli et al., Nat Biotechnol. 31(10): 928-933 (2013); Tsukahara et al., Biochem Biophys Res Commun 438(1): 84-9 (2013); and Davila et al., PLoS ONE 8(4): e61338 (2013). These methods may be used in connection with the methods and compositions provided herein.

In some embodiments, the cell therapy (e.g., adoptive T cell therapy) is carried out by autologous transfer, in which the cells are isolated and/or otherwise prepared from the subject who is to receive the cell therapy, or from a sample derived from such a subject. Thus, in some aspects, the cells are derived from a subject in need of a treatment and the cells, following isolation and processing are administered to the same subject. In other embodiments, the cell therapy (e.g., adoptive T cell therapy) is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. The subject can be male or female and can be any suitable age, including infant, juvenile, adolescent, adult, and geriatric subjects. In some examples, the subject is a validated animal model for disease, adoptive cell therapy, and/or for assessing toxic outcomes.

The composition provided herein can be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration.

The amount of a prophylactic or therapeutic agent provided herein that will be effective in the prevention and/or treatment of a disease or condition can be determined by standard clinical techniques. Effective doses may be extrapolated from dose-response curves derived from in vitro or animal model test systems. For the prevention or treatment of disease, the appropriate dosage of the binding molecule or cell may depend on the type of disease or disorder to be treated, the type of binding molecule, the severity and course of the disease or disorder, whether the therapeutic agent is administered for preventive or therapeutic purposes, previous therapy, the patient's clinical history and response to the agent, and the discretion of the attending physician. The compositions, molecules and cells are in some embodiments suitably administered to the patient at one time or over a series of treatments. Multiple doses may be administered intermittently. An initial higher loading dose, followed by one or more lower doses may be administered.

In the context of genetically engineered cells, in some embodiments, a subject may be administered the range of about one million to about 100 billion cells and/or that amount of cells per kilogram of body weight. In some embodiments, wherein the pharmaceutical composition comprises any one of the engineered immune cells described herein, the pharmaceutical composition is administered at a dosage of at least about any of 10⁴, 10⁵, 10⁶, 10⁷, 10⁸, or 10⁹ cells/kg of body weight of the individual. Dosages may vary depending on attributes particular to the disease or disorder and/or patient and/or other treatments.

In some embodiments, the pharmaceutical composition is administered for a single time. In some embodiments, the pharmaceutical composition is administered for multiple times (such as any of 2, 3, 4, 5, 6, or more times). In some embodiments, the pharmaceutical composition is administered once or multiple times during a dosing cycle. A dosing cycle can be, e.g., 1, 2, 3, 4, 5 or more week(s), or 1, 2, 3, 4, 5, or more month(s). The optimal dosage and treatment regime for a particular patient can be determined by one skilled in the art of medicine by monitoring the patient for signs of disease and adjusting the treatment accordingly.

In some embodiments, the compositions provided herein are administered as part of a combination treatment, such as simultaneously with or sequentially with, in any order, another therapeutic intervention, such as another antibody or engineered cell or receptor or agent, such as a cytotoxic or therapeutic agent.

In some embodiments, the compositions provided herein are co-administered with one or more additional therapeutic agents or in connection with another therapeutic intervention, either simultaneously or sequentially in any order. In some embodiments, the cells are co-administered with another therapy sufficiently close in time such that the cell populations enhance the effect of one or more additional therapeutic agents, or vice versa. In some embodiments, the compositions provided herein are administered prior to the one or more additional therapeutic agents. In some embodiments, the compositions provided herein are administered after to the one or more additional therapeutic agents.

In certain embodiments, once the cells are administered to a mammal (e.g., a human), the biological activity of the engineered cell populations is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD107a, IFNγ, IL-2, and TNF. In some aspects the biological activity is measured by assessing clinical outcome, such as reduction in tumor burden or load.

### 5.8. Kits and Articles of Manufacture

Further provided are kits, unit dosages, and articles of manufacture comprising any of the engineered immune effector cells described herein. In some embodiments, a kit is provided which contains any one of the pharmaceutical compositions described herein and preferably provides instructions for its use.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer) described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

The disclosure is generally disclosed herein using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

A number of embodiments of the disclosure have been described. Nevertheless, it will be understood that various modifications may be made without departing from the spirit and scope of the disclosure. Accordingly, the following examples are intended to illustrate but not limit the scope of disclosure described in the claims.

### 6. EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### 6.1. Example 1—Exemplary T Cell and APC Cell Engagers (TALEs) and Exemplary Methods

T cell and APC cells engagers (TALEs) were designed and their functions were tested herein. Schematic illustration of physical proximity-induced mutual beneficial interaction between T (or TCR-T or CAR-T) cells and APC cells is shown in FIGS. 1A-1B. Specifically, T (or TCR-T or CAR-T) cells benefit from APC cells with co-stimulatory signals derived from co-stimulatory receptor as well as cytokines (FIG. 1A). In the present disclosure, a specific mobilization signal is introduced from T (or TCR-T or CAR-T) cell to APC cells (FIG. 1B). This mobilization ligand/binder (TALE) can facilitate the interaction between T (or TCR-T or CAR-T) cells and APC cells. The engagement between T (or TCR-T or CAR-T) and APC cells stimulates APC-induced T (or TCR-T or CAR-T) cell expansion, and reciprocally, T (or TCR-T or CAR-T) cell derived armors can further mobilize and maturate APC cells, which results in positive feedback between T (or TCR-T/CAR-T) and APC cells.

The TALE design may be applied through following non-limiting exemplary receptors or ligands: CD40, CLL1, FLT3, FLT3L, 4-1BB, 4-1BBL, GITR, GITRL, CD27, CD70, OX40, OX40L, PD-1, PD-L1, PD-L2, Galectin-9, B7-H3, B7-H4, ICAM1, ICOS, ICOSL, CD30, CD30L, TIM1, TIM3, TIM4, SEMA4A, CD155, TIGIT, CD160, CD28, CD80, CD86, CTLA4, LAG3, LFA-1, LTβR, and HVEM, several of which were specifically examined in the following examples.

The exemplary methods for conducting the studies in the following examples are illustrated in this example.

In Vitro Study Protocol:

To demonstrate the role of TALEs, a CAR targeting GPC2 was selected as an example in the following studies.

To analyze the mutual beneficial interaction between T (or TCR-T or CAR-T) cells and APCs, undifferentiated monocytes were used as APCs. Undifferentiated monocytes maintain minimal activation signals, therefore, the base proliferation benefit to T cells is minimized. Low activated monocyte can be readily stimulated by T (or TCR-T or CAR-T) cell derived mobilization signal, if presented, therefore activation of monocytes can be maximally recorded.

In the following proof-of-concept studies, CAR-T cells and monocytes were co-cultured to dissect the bi-directional functional interaction between adoptive transferred T cells and APC cells.

Generation of CAR-T Cells:

T cells were isolated from healthy donor PBMCs (HemaCare) using pan T cell isolation kit (Miltenyi Biotec, 130096535). Isolated T cells were cultured under AIMV (Gibco, 31035025) medium with 5% FBS (Gibco, 10099141) and further activated by CD3/CD28 activation beads (Miltenyi Biotec, 130091442) at a 2:1 ratio in the 37° C., 5% CO₂ incubator. 24 or 72 hours after initial activation, T cells were transduced with lentivirus expressing a GPC2 targeting CAR at multiplicity of infection (MOI) of 5 in the presence of 8 pg/ml polybrene (SIGMA-ALDRICH, H9268-10G). Additional IL-2 was supplemented to a final concentration of 300 IU/ml. Fresh medium was replaced 24 hours post lentiviral infection. Infected T cells were maintained under AIMV medium with 5% FBS and 300 IU/ml IL-2 at a cell density between 5E+05 to 1E+06 cells/mi.

CAR expression was determined at 4 days post infection by a rabbit Anti-VHH antibody (Genscript) via flow cytometry (BD FACsCelesta). CAR positive rate and geometric mean expression (mean fluorescent intensity, MFI) was further analyzed by Flowjo 7.6.

Monocyte Isolation:

Briefly, monocytes were isolated from the PBMCs from the same healthy donor as CAR-T preparation by using human CD14 microbeads ultrapure kit (Miltenyi Biotec, 130-118-906).

CAR-T Cell Labeling and Co-Culturing with Monocyte:

Day 5 post viral infection, CAR-T cells were labelled with 2 μM CFSE (DOJINDO, C375) and co-culture with freshly isolated monocyte at a ratio of monocyte: T=1:1 in 24-well plates (Corning). To minimize the effect of cytokine (s) in T cell proliferation and monocyte activation, basic AIMV medium supplemented with 5% FBS was used during co-culture to study the homeostatic proliferation of CAR-T cells and activation of monocyte.

48 hours after incubation, monocyte activation was monitored by surface expression of CD14, CD80, CD83 and CD86 via flow cytometry. 72 hours post co-culture, homeostatic proliferation of CAR-T cells was visualized by a decrease of CFSE signal intensity (mean fluorescent intensity, MFI). Proliferation of total T cells (mixture of CAR⁺ and CAR⁻ T cells) as well as CAR⁺ T cells was analyzed separately.

A proliferation index was employed to calculate the monocyte induced proliferation with following formula.

Proliferation index of total T cells=MFI _{(total T alone)}−MFI _{(monocyte and total T co-culture) )}/MFI _{(total T alone)}×100

Proliferation index of CAR⁺T cells=MFI _{(CAR}⁺T alone)−MFI_{(monocyte and CAR}⁺T co-culture)/MFI (CAR⁺T alone)×100

### 6.2. Example 2—T Cells Armored with LTα/β and/or LIGHT Show Enhanced Proliferation when Co-Cultured with Monocyte

Exemplary TALEs (i.e., based on LTα/β or LIGHT) were constructed in this example. Primary structure and molecular design of CAR armored with LTα/β and CAR armored with LIGHT is illustrated in FIG. 2A. TALE-induced physical proximity between T cells and APC is shown in FIG. 2B.

The sequences of various regions of the exemplary constructs are shown in the table below.

TABLE 3
Sequences of the Components of the Examplary Polypeptides
Comprising LTα/β or LIGHT

| SEQ | | |
|---|---|---|
| ID | Component of | |
| NO: | Armor | Sequence |
| 2 | P2A element | GSGATNFSLLKQAGDVEENPGP |
| 3 | CD8α signal | MALPVTALLLPLALLLHAARP |
| | peptide | |
| 4 | CD8α hinge | TTTPAPRPPTPAPTIASQPLSLRPEACRPAAGGAVHTRGLDFA |
| | | CD |
| 5 | CD8α trans- | IYIWAPLAGTCGVLLLSLVITLYC |
| | membrane | |
| 6 | LTα signal | MTPPERLFLPRVCGTTLHLLLLGLLLVLLPGAQG |
| | peptide | |
| 7 | LTα | MTPPERLFLPRVCGTTLHLLLLGLLLVLLPGAQGLPGVGLTP |
| | | SAAQTARQHPKMHLAHSTLKPAAHLIGDPSKQNSLLWRANT |
| | | DRAFLQDGFSLSNNSLLVPTSGIYFVYSQVVFSGKAYSPKAT |
| | | SSPLYLAHEVQLFSSQYPFHVPLLSSQKMVYPGLQEPWLHS |
| | | MYHGAAFQLTQGDQLSTHTDGIPHLVLSPSTVFFGAFAL |
| 8 | LTα mutant 1 | KPAAHLIGDPSKQNSLLWRANTDRAFLQDGFSLSNNSLLVPT |
| | | SGIYFVYSQVVFSGKAYSPKATSSPLYLAHEVQLFSSQYPFH |
| | | VPLLSSQKMVYPGLQEPWLHSMYHGAAFQLTQGDQLSTHT |
| | | DGIPHLVLSPSTVFFGAFAL |
| 9 | LTα mutant 2 | LPGVGLTPSAAQTARQHPKMHLAHSTLKPAAHLIGDPSKQN |
| | | SLLWRANTDRAFLQDGFSLSNNSLLVPTSGIYFVYSQVVFSG |
| | | KAYSPKATSSPLYLAHEVQLFSSQYPFHVPLLSSQKMVYPGL |
| | | QEPWLHSMYHGAAFQLTQGDQLSTHTDGIPHLVLSPSTVFF |
| | | GAFAL |
| 10 | LTβ | MGALGLEGRGGRLQGRGSLLLAVAGATSLVTLLLAVPITVL |
| | | AVLALVPQDQGGLVTETADPGAQAQQGLGFQKLPEEEPETD |
| | | LSPGLPAAHLIGAPLKGQGLGWETTKEQAFLTSGTQFSDAEG |
| | | LALPQDGLYYLYCLVGYRGRAPPGGGDPQGRSVTLRSSLYR |
| | | AGGAYGPGTPELLLEGAETVTPVLDPARRQGYGPLWYTSVG |
| | | FGGLVQLRRGERVYVNISHPDMVDFARGKTFFGAVMVG |
| 11 | LTβ mutant 1 | QDQGGLVTETADPGAQAQQGLGFQKLPEEEPETDLSPGLPA |
| | | AHLIGAPLKGQGLGWETTKEQAFLTSGTQFSDAEGLALPQD |
| | | GLYYLYCLVGYRGRAPPGGGDPQGRSVTLRSSLYRAGGAY |
| | | GPGTPELLLEGAETVTPVLDPARRQGYGPLWYTSVGFGGLV |
| | | QLRRGERVYVNISHPDMVDFARGKTFFGAVMVG |
| 12 | LTβ mutant 2 | LPAAHLIGAPLKGQGLGWETTKEQAFLTSGTQFSDAEGLALP |
| | | QDGLYYLYCLVGYRGRAPPGGGDPQGRSVTLRSSLYRAGG |
| | | AYGPGTPELLLEGAETVTPVLDPARRQGYGPLWYTSVGFGG |
| | | LVQLRRGERVYVNISHPDMVDFARGKTFFGAVMVG |
| 13 | Engineered | KPAAHLIGDPSKQNSLLWRANTDRAFLQDGFSLSNNSLLVPT |
| | mutant LTα/β 1 | SGIYFVYSQVVFSGKAYSPKATSSPLYLAHEVQLFSSQYPFH |
| | | VPLLSSQKMVYPGLQEPWLHSMYHGAAFQLTQGDQLSTHT |
| | | DGIPHLVLSPSTVFFGAFALGGSGLPAAHLIGAPLKGQGLGW |
| | | ETTKEQAFLTSGTQFSDAEGLALPQDGLYYLYCLVGYRGRA |
| | | PPGGGDPQGRSVTLRSSLYRAGGAYGPGTPELLLEGAETVTP |
| | | VLDPARRQGYGPLWYTSVGFGGLVQLRRGERVYVNISHPD |
| | | MVDFARGKTFFGAVMVGGGSGLPAAHLIGAPLKGQGLGWE |
| | | TTKEQAFLTSGTQFSDAEGLALPQDGLYYLYCLVGYRGRAP |
| | | PGGGDPQGRSVTLRSSLYRAGGAYGPGTPELLLEGAETVTP |
| | | VLDPARRQGYGPLWYTSVGFGGLVQLRRGERVYVNISHPD |
| | | MVDFARGKTFFGAVMVG |
| 14 | Engineered | MTPPERLFLPRVCGTTLHLLLLGLLLVLLPGAQGLPGVGLTP |
| | mutant LTα/β 2 | SAAQTARQHPKMHLAHSTLKPAAHLIGDPSKQNSLLWRANT |
| | | DRAFLQDGFSLSNNSLLVPTSGIYFVYSQVVFSGKAYSPKAT |
| | | SSPLYLAHEVQLFSSQYPFHVPLLSSQKMVYPGLQEPWLHS |
| | | MYHGAAFQLTQGDQLSTHTDGIPHLVLSPSTVFFGAFALGG |
| | | GGSQDQGGLVTETADPGAQAQQGLGFQKLPEEEPETDLSPG |
| | | LPAAHLIGAPLKGQGLGWETTKEQAFLTSGTQFSDAEGLALP |
| | | QDGLYYLYCLVGYRGRAPPGGGDPQGRSVTLRSSLYRAGG |
| | | AYGPGTPELLLEGAETVTPVLDPARRQGYGPLWYTSVGFGG |
| | | LVQLRRGERVYVNISHPDMVDFARGKTFFGAVMVGGGGGS |
| | | QDQGGLVTETADPGAQAQQGLGFQKLPEEEPETDLSPGLPA |
| | | AHLIGAPLKGQGLGWETTKEQAFLTSGTQFSDAEGLALPQD |
| | | GLYYLYCLVGYRGRAPPGGGDPQGRSVTLRSSLYRAGGAY |
| | | GPGTPELLLEGAETVTPVLDPARRQGYGPLWYTSVGFGGLV |
| | | QLRRGERVYVNISHPDMVDFARGKTFFGAVMVG |
| 15 | Engineered | MGALGLEGRGGRLQGRGSLLLAVAGATSLVTLLLAVPITVL |
| | mutant LTα/ß 3 | AVLALVPQDQGGLVTETADPGAQAQQGLGFQKLPEEEPETD |
| | | LSPGLPAAHLIGAPLKGQGLGWETTKEQAFLTSGTQFSDAEG |
| | | LALPQDGLYYLYCLVGYRGRAPPGGGDPQGRSVTLRSSLYR |
| | | AGGAYGPGTPELLLEGAETVTPVLDPARRQGYGPLWYTSVG |
| | | FGGLVQLRRGERVYVNISHPDMVDFARGKTFFGAVMVGGG |
| | | GGSQDQGGLVTETADPGAQAQQGLGFQKLPEEEPETDLSPG |
| | | LPAAHLIGAPLKGQGLGWETTKEQAFLTSGTQFSDAEGLALP |
| | | QDGLYYLYCLVGYRGRAPPGGGDPQGRSVTLRSSLYRAGG |
| | | AYGPGTPELLLEGAETVTPVLDPARRQGYGPLWYTSVGFGG |
| | | LVQLRRGERVYVNISHPDMVDFARGKTFFGAVMVGGGGGS |
| | | LPGVGLTPSAAQTARQHPKMHLAHSTLKPAAHLIGDPSKQN |
| | | SLLWRANTDRAFLQDGFSLSNNSLLVPTSGIYFVYSQVVFSG |
| | | KAYSPKATSSPLYLAHEVQLFSSQYPFHVPLLSSQKMVYPGL |
| | | QEPWLHSMYHGAAFQLTQGDQLSTHTDGIPHLVLSPSTVFF |
| | | GAFAL |
| 16 | Engineered | MGALGLEGRGGRLQGRGSLLLAVAGATSLVTLLLAVPITVL |
| | mutant LTα/β 4 | AVLALVPQDQGGLVTETADPGAQAQQGLGFQKLPEEEPETD |
| | | LSPGLPAAHLIGAPLKGQGLGWETTKEQAFLTSGTQFSDAEG |
| | | LALPQDGLYYLYCLVGYRGRAPPGGGDPQGRSVTLRSSLYR |
| | | AGGAYGPGTPELLLEGAETVTPVLDPARRQGYGPLWYTSVG |
| | | FGGLVQLRRGERVYVNISHPDMVDFARGKTFFGAVMVGGG |
| | | GGSLPGVGLTPSAAQTARQHPKMHLAHSTLKPAAHLIGDPS |
| | | KQNSLLWRANTDRAFLQDGFSLSNNSLLVPTSGIYFVYSQV |
| | | VFSGKAYSPKATSSPLYLAHEVQLFSSQYPFHVPLLSSQKMV |
| | | YPGLQEPWLHSMYHGAAFQLTQGDQLSTHTDGIPHLVLSPS |
| | | TVFFGAFALGGGGSQDQGGLVTETADPGAQAQQGLGFQKL |
| | | PEEEPETDLSPGLPAAHLIGAPLKGQGLGWETTKEQAFLTSG |
| | | TQFSDAEGLALPQDGLYYLYCLVGYRGRAPPGGGDPQGRSV |
| | | TLRSSLYRAGGAYGPGTPELLLEGAETVTPVLDPARRQGYG |
| | | PLWYTSVGFGGLVQLRRGERVYVNISHPDMVDFARGKTFFG |
| | | AVMVG |
| 17 | LIGHT | MEESVVRPSVFVVDGQTDIPFTRLGRSHRRQSCSVARVGLGL |
| | | LLLLMGAGLAVQGWELLQLHWRLGEMVTRLPDGPAGSWE |
| | | QLIQERRSHEVNPAAHLTGANSSLTGSGGPLLWETQLGLAFL |
| | | RGLSYHDGALVVTKAGYYYIYSKVQLGGVGCPLGLASTITH |
| | | GLYKRTPRYPEELELLVSQQSPCGRATSSSRVWWDSSFLGG |
| | | VVHLEAGEKVVVRVLDERLVRLRDGTRSYFGAFMV |
| 18 | CD28 hinge | KIEVMYPPPYLDNEKSNGTIIHVKGKHLCPSPLFPGPSKP |
| 19 | CD28 trans- | FWVLVVVGGVLACYSLLVTVAFIIFWV |
| | membrane | |
| 20 | Membrane | MTPPERLFLPRVCGTTLHLLLLGLLLVLLPGAQGLPGVGLTP |
| | anchored | SAAQTARQHPKMHLAHSTLKPAAHLIGDPSKQNSLLWRANT |
| | engineered | DRAFLQDGFSLSNNSLLVPTSGIYFVYSQVVFSGKAYSPKAT |
| | mutant LTα/β | SSPLYLAHEVQLFSSQYPFHVPLLSSQKMVYPGLQEPWLHS |
| | | MYHGAAFQLTQGDQLSTHTDGIPHLVLSPSTVFFGAFALGG |
| | | GGSQDQGGLVTETADPGAQAQQGLGFQKLPEEEPETDLSPG |
| | | LPAAHLIGAPLKGQGLGWETTKEQAFLTSGTQFSDAEGLALP |
| | | QDGLYYLYCLVGYRGRAPPGGGDPQGRSVTLRSSLYRAGG |
| | | AYGPGTPELLLEGAETVTPVLDPARRQGYGPLWYTSVGFGG |
| | | LVQLRRGERVYVNISHPDMVDFARGKTFFGAVMVGGGGGS |
| | | QDQGGLVTETADPGAQAQQGLGFQKLPEEEPETDLSPGLPA |
| | | AHLIGAPLKGQGLGWETTKEQAFLTSGTQFSDAEGLALPQD |
| | | GLYYLYCLVGYRGRAPPGGGDPQGRSVTLRSSLYRAGGAY |
| | | GPGTPELLLEGAETVTPVLDPARRQGYGPLWYTSVGFGGLV |
| | | QLRRGERVYVNISHPDMVDFARGKTFFGAVMVGGGGGSKI |
| | | EVMYPPPYLDNEKSNGTIIHVKGKHLCPSPLFPGPSKPFWVL |
| | | VVVGGVLACYSLLVTVAFIIFWV |
| 25 | anti-GPC2 | EVQLVESGGGLVQPGGSLRLSCAISEFTYKNTCVGWFRQAP |
| | sdAb | GKGREGVAAIDSDGNTNYVDSVKGRFTISQDNSKNTVYLQ |
| | | MNSLRAEDTAMYYCAAGAYCGRLLLWIGNYAYWGQGTLV |
| | | TVSS |
| 26 | 4-1BB co- | KRGRKKLLYIFKQPFMRPVQTTQEEDGCSCRFPEEEEGGCEL |
| | stimulatroy | |
| | domain | |
| 27 | CD3z | RVKFSRSADAPAYKQGQNQLYNELNLGRREEYDVLDKRRG |
| | | RDPEMGGKPRRKNPQEGLYNELQKDKMAEAYSEIGMKGER |
| | | RRGKGHDGLYQGLSTATKDTYDALHMQALPPR |

Briefly, 5 days post viral infection, conventional (without TALE) or armored CAR-T cells were adjusted with an untransduced T (unT) to CAR⁺ ratio of 20% and co-cultured with monocyte for 72 hours to analyze of homeostatic proliferation of T cells. Interestingly, monocyte can induce potent homeostatic proliferation of both CAR-T cells and unT cells as reflected by positive value of the proliferation index. More importantly, an increase of proliferation by about 10% was founded in CAR-T cells armored with wild-type LTα/β (tandem wild-type LTα and wild-type LTβ via P2A) or LIGHT in comparison to conventional CAR-T cells, indicating that LTα/β and/or LIGHT based armor strategy can potentially increase CAR-T cell proliferation (FIG. 3A). Moreover, further analysis of proliferation in CAR⁺ T cells confirmed an increase of proliferation of CAR⁺ T (FIG. 3B). More specifically, CAR⁺ T cells armored with LIGHT derived TALE proliferate even more comparing to LTα/β. It has been known that, while LTα/β can specifically interact with LTβR, LIGHT can potently bind to both LTβR and HVEM. Thus, the increased proliferation of LIGHT in comparison to LTα/β might be a synergistic outcome from both LTβR and HVEM. We chose several of the above CAR T cells for further evaluation below, among them, the structures of CARs containing CD8α signaling-anti-GPC2 binder-CD8α hinge-CD8α transmembrane-4-1BB co-stimulatory domain-CD3z.

### 6.3. Example 3—CAR-T Cells Armored with LTα/β and/or LIGHT Promote Monocyte Activation

Our strategic approach aimed in part at bi-directional mutual beneficial interaction. Thus, monocyte activation, such as down-regulation of CD14, up-regulation of CD40, CD80, CD83 and CD86, was monitored. In line with increased proliferation, a general higher activation pattern was observed in monocytes when co-cultured with CAR-T cells armored with wild-type LTα/β or LIGHT TALEs in comparison to conventional CAR-T cells (FIG. 4). Interestingly, consistent with a more potent increase of proliferation in LIGHT armor, a relative higher activation status was observed in monocytes co-cultured with LIGHT armored CAR-T in comparison to wild-type LTα/β armored CAR-T cells (FIG. 4).

Taken together, our results of CAR-T cell proliferation and monocyte activation confirmed that LTα/β and/or LIGHT derived TALEs can potently induce bi-directional mutual beneficial interaction between T and APC cells.

### 6.4. Example 4—Increased Proliferation of CAR-T Cells Armored with LTα/β and/or LIGHT In Vivo

To further demonstrate the proliferation enhancing effect of wild-type LTα/β and/or LIGHT derived armors in CAR-T cells, a CAR targeting GPC2 protein was selected in the following proof of concept in vivo study. NCG mice (NOD-Prkdc Cd5 Il2rg Cd/NjuCrl) were subcutaneously injected with neuroblastoma SH-SY5Y cells. A single dose of untransduced T cells (2.5×10⁶) or CAR T cells (5×10⁵) was administered intravenously to tumor engrafted mice 14 days after tumor inoculation. CAR-T proliferation in peripheral blood were monitored once a week for 4 weeks. In line with increased proliferation in vitro, a notable increase of CD3 T cells (FIG. 5A) as well as CAR⁺ T cells (FIG. 5B) were detected at day 14 post infusion, confirming a beneficial effect of LTα/β and/or LIGHT based TALEs in promoting CAR-T cell proliferation.

Additionally, an end-point analysis of T cells in freshly harvested tumors as well as physiological tissues further demonstrated higher percentage of CD3-T and CAR-T cells (FIG. 6). Interestingly, although increased proliferation in peripheral blood (PB) was comparable between wild-type LTα/β and LIGHT based TALEs, a notable difference of CD3 and CAR-T cell in organs, more specifically in tumors, was found between wild-type LTα/β and LIGHT based TALEs (FIG. 6).

Taken together, these observations suggested that CAR-T cells armored LTα/β or LIGHT TALEs can strikingly boost their in vivo proliferation both in peripheral blood, organs as well as tumors.

### 6.5. Example 5—CAR-T Cells Armored with LTα/β and/or LIGHT Show Superior Anti-Tumor Efficacy in Comparison to Conventional CAR-T Cells

To further correlate the CAR-T proliferation and in vivo anti-tumor efficacy, tumor growth was also monitored. Tumor length (L) and width (W) was measured by caliper every 3-4 days after CAR T cells treatment. Tumor volume was estimated using formula: V=(W²×L)/2. Fourteen days after treatment, the NCG mice treated with CAR-T cells all showed reduced tumor burden comparing with the untransduced T cell-treated group (FIG. 7A). In line with enhanced proliferation in vivo, comparison to conventional naked CAR-T cells, the anti-tumor efficacy was more pronounced in CAR-T cell armored wild-type LTα/β TALE (100%, 4/4 mice) (FIGS. 7B and 7C). Interestingly, in full agreement with difference of T cell proliferation in tumors, CAR-T cells armored with LIGHT showed shown partially efficacy (50%, 2/4 mice)(FIGS. 7B and 7D). Collectively, these results strikingly suggested the superiority of armored CAR T cells in tumor regression and protection of mice from disease progression.

### 6.6. Example 6—CAR-T Cells Armored with LTα/β and/or LIGHT are Well Tolerated In Vivo

Increased in vivo proliferation as well as tumor repressive efficacy may lead to undesired overexpansion, which may then lead to lethal cytokine release syndrome. Thus, the potential side-effect derived from wild-type LTα/β and/or LIGHT based TALEs was examined. Firstly, the animal status and body weight were closely monitored twice a week. Notably, no striking changes were found in mice infused with wild-type LTα/β or LIGHT armored CAR-T cells (FIG. 8A). Similarly, observations of animal behaviors revealed no difference between groups of mice treated with conventional CAR-T cells or wild-type LTα/β or LIGHT armored CAR-T cells. Furthermore, analysis of tissues in the end-point necropsy confirmed no difference between mice treated with conventional CAR-T cells or wild-type LTα/β or LIGHT armored CAR-T cells (FIG. 8B). Together, these findings demonstrated a well-tolerated profile of wild-type LTα/β and/or LIGHT based TALEs in vivo.

### 6.7. Example 7—CAR-T Cells Armored with Engineered Mutant LTα/β Show Superior Anti-Tumor Efficacy in Comparison to Wild-Type LTα/β Armored CAR-T Cells

We further explored the anti-tumor activity of CAR-T cells armored with engineered mutant LTα/0 comprising amino acid sequence of SEQ ID NO. 14 in comparison to CAR-T cells armored with wild-type LT/s. Tumor length (L) and width (W) was measured by caliper every 3-4 days after CAR T cells treatment. Tumor volume was estimated using formula: V=(W²×L)/2. Interestingly, comparison to CAR-T cells armored with wild-type LTα/β, the anti-tumor efficacy was more pronounced in CAR-T cell armored engineered mutant LTα/β TALE (FIG. 9). Collectively, these results strikingly suggested the superiority of engineered mutant LTα/β TALE in promoting CAR-T function during tumor regression and protection of mice from disease progression.

### 6.8. Example 8—A Mutual Beneficial Interaction Between Monocyte and CAR-T Cell Armored with TALEs to APC Activation Receptors

We next explored the beneficial interaction between adoptive transferred T cell and APC via activating receptors expressed on APC. CD40 was selected as an exemplary activating receptor on APC.

Primary structure and molecular design of CAR armored with CD40 TALEs is illustrated in FIG. 10A. Armored-mediated physical proximity between T cells and APC is shown in FIG. 10B.

The sequences of P2A (SEQ ID NO: 2), CD8α signal peptide (SEQ ID NO: 3), CD8α hinge region (SEQ ID NO: 4), CD8α transmembrane region (SEQ ID NO: 5), anti-GPC2 sdAb (SEQ ID NO: 25), 4-1BB co-stimulatory domain (SEQ ID NO: 26), and CD3z intracellular region (SEQ ID NO. 27) in the CAR in the present example are shown in Table 3 above. The sequences of other various regions of the exemplary constructs in the present example are shown in the table below.

TABLE 4
Sequences of the Components of the Examplary Polypeptides
Comprising CD40 Binding Domains

| SEQ ID | Component of | |
|---|---|---|
| NO: | Armor | Sequence |
| 18 | CD28 hinge | KIEVMYPPPYLDNEKSNGTIIHVKGKHLCPSPLFPGPS |
| | | KP |
| 19 | CD28 trans- | FWVLVVVGGVLACYSLLVTVAFIIFWV |
| | membrane | |
| 21 | CD40 antibody | DIQLQQSGPGLVKPSQSLSLTCSVTGYSITTNYNWNW |
| | (G28.5) | IRQFPGNKLEWMGYIRYDGTSEYTPSLKNRVSITRDT |
| | | SMNQFFLRLTSVTPEDTATYYCARLDYWGQGTLVT |
| | | VSSGGGGSGGGGSGGGGSDIVMTQNPLSLPVSLGDE |
| | | ASISCRSSQSLENSNGNTFLNWFFQKPGQSPQLLIYRV |
| | | SNRFSGVPDRFSGSGSGTDFTLKISRVEAEDLGVYFC |
| | | LQVTHVPYTFGGGTTLEIK |
| 24 | CD40 ligand | MIETYNQTSPRSAATGLPISMKIFMYLLTVFLITQMIG |
| | | SALFAVYLHRRLDKIEDERNLHEDFVFMKTIQRCNT |
| | | GERSLSLLNCEEIKSQFEGFVKDIMLNKEETKKENSFE |
| | | MQKGDQNPQIAAHVISEASSKTTSVLQWAEKGYYT |
| | | MSNNLVTLENGKQLTVKRQGLYYIYAQVTFCSNREA |
| | | SSQAPFIASLCLKSPGRFERILLRAANTHSSAKPCGQQ |
| | | SIHLGGVFELQPGASVFVNVTDPSQVSHGTGFTSFGL |
| | | LKL |

CAR-T cells armored with CD40 TALEs (i.e., either a reported antibody (G28.5)-derived single chain Fv or a natural wild-type CD40 ligand) were generated. The interplay between monocytes and CAR-T cells were explored. Notably, an increased proliferation of total T cells (FIG. 11A) as well as CAR⁺ T cells (FIG. 11B) was visualized in CAR-T cells armored with either CD40 antibody or CD40L TALE. Reciprocally, CAR-T cell-mediated activation of monocyte was more visible in CAR-T cells armored with CD40 TALEs in comparison to conventional CAR-T cells (FIG. 12). It is notable that CD40 antibody based TALE could potently induce down-regulation of CD40 expression in monocyte, most probably via promoting its endocytosis upon binding.

### 6.9. Example 9—A Mutual Beneficial Interaction Between Monocyte and CAR-T Cell Armored with TALEs to APC Inhibitory Receptors

We further explored the possibility of beneficial interaction between adoptive transferred T cell and APC via inhibitory receptors expressed on APC. CLL1 (CLEC12A) was selected as an exemplary inhibitory receptor on APC. CLL1 comprises an intracellular inhibitory signal derived from its ITIM motif.

Primary structure and molecular design of CAR armored with CLL1 antibody TALEs is illustrated in FIG. 13A. Armored-mediated physical proximity between T cells and APC is shown in FIG. 13B.

The sequences of P2A (SEQ ID NO: 2), CD8α signal peptide (SEQ ID NO: 3), CD8α hinge region (SEQ ID NO: 4), CD8α transmembrane region (SEQ ID NO: 5), anti-GPC2 sdAb (SEQ ID NO: 25), 4-1BB co-stimulatory domain (SEQ ID NO: 26), and CD3z intracellular region (SEQ ID NO: 27) in the CAR in the present example are shown in Table 3 above. The sequences of other various regions (particularly in the additional domain) of the exemplary constructs in the present example are shown in the table below.

TABLE 5
Sequences of the Components of the Examplary Polypeptides
Comprising CLL1 Binding Domains

| SEQ ID | Component of | |
|---|---|---|
| NO: | Armor | Sequence |
| 4 | CD8α hinge | TTTPAPRPPTPAPTIASQPLSLRPEACRPAAGGAVHTR |
| | | GLDFACD |
| 5 | CD8α trans- | IYIWAPLAGTCGVLLLSLVITLYC |
| | membrane | |
| 22 | CLL1 binder | DIQLTQSPSSLSASVGDRVSFTCQASQDINNFLNWYQ |
| | (Kite) | QKPGKAPKLLIYDASNLETGVPSRFSGSGSGTDFTFTI |
| | | SSLQPEDIATYYCQQYGNLPFTFGGGTKVEIKRGGGG |
| | | SGGGGSGGGGSQVQLQESGPGLVKPSETLSLTCTVS |
| | | GGSISSYYWSWIRQPPGKGLEWIGYIYYSGSTNYNPS |
| | | LKSRVTISVDTSKNQFSLKLSSVTAADTAVYYCVSLV |
| | | YCGGDCYSGFDYWGQGTLVTVSS |
| 23 | CLL1 binder | DIQMTQSPSSLSASVGDRVTITCRASQSVSTSSYNYM |
| | (iCell) | HWYQQKPGKPPKLLIKYASNLESGVPSRFSGSGSGTD |
| | | FTLTISSLQPEDFATYYCQHSWEIPLTFGQGTKVEIKG |
| | | GGGSGGGGSGGGGSEVQLVQSGAEVKKPGASVKVS |
| | | CKASGYSFTDYYMHWVRQAPGQGLEWIGRINPYNG |
| | | AAFYSQNFKDRVTLTVDTSTSTAYLELSSLRSEDTAV |
| | | YYCAIERGADLEGYAMDYWGQGTLVTVSS |

CAR-T cells armored with antibody based CLL1 TALEs were generated. Since no well-known ligand has been discovered for CLL1, an antibody based armor design was explored in this study. Two clinical binder from Kite (Ab-Kite) and iWell gene (Ab-iCell) were tested. The interplay between monocytes and CAR-T cells were investigated. Interestingly, an increased proliferation of total T cells (FIG. 14A) as well CAR⁺ T cells (FIG. 14B) was visualized in CAR-T cells armored with either antibody based TALEs. Reciprocally, CAR-T cell-mediated activation of monocyte was more visible in CAR-T cells with CLL1-TALEs in comparison to conventional CAR-T cells (FIG. 15).

These observations strongly suggest that engagers even targeting to inflammatory inhibitory receptor or non-stimulatory structural receptors may induce bi-directional mutual beneficial interaction by inducing a physical proximity between APC cells and T cells.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

1. A polypeptide comprising:
(a) a chimeric antigen receptor (CAR) comprising (i) an extracellular domain capable of binding to a first antigen, (ii) a transmembrane domain, and (iii) an intracellular domain; and
(b) a domain capable of binding to a second antigen expressed on the surface of a cell that can interact with a T cell,
wherein the CAR and the domain are fused by a peptide linker.

2. The polypeptide of claim 1, wherein the cell that can interact with the T cell is capable of presenting the first antigen to the T cell and/or inducing a response from the T cell upon interaction.

3. The polypeptide of claim 1, wherein the cell that can interact with the T cell is selected from a group consisting of macrophage, dendritic cell, B lymphocyte (B cell), mast cell, basophil, eosinophil, group 3 innate lymphoid cell (ILC3), monocyte, neutrophil, natural killer cell, fibroblastic reticular cell, endothelial cell, pericyte, epithelial cell, fibroblast and artificial APC cell (aAPC).

4. The polypeptide of claim 1, where the cell that can interact with the T cell expresses a MHC molecule.

5. The polypeptide of claim 4, wherein the MHC molecule is a MHC class I molecule or MHC class II molecule.

6.-8. (canceled)

9. The polypeptide of claim 1, wherein the second antigen is a receptor or ligand expressed on the cell that can interact with the T cell.

10. The polypeptide of claim 1, wherein the first antigen is tumor associated antigen, and/or the second antigen is selected from a group consisting of CD40, CLL1, FLT3, FLT3L, 4-1BB, 4-1BBL, GITR, GITRL, CD27, CD70, OX40, OX40L, PD-1, PD-L1, PD-L2, Galectin-9, B7-H3, B7-H4, ICAM1, ICOS, ICOSL, CD30, CD30L, TIM1, TIM3, TIM4, SEMA4A, CD155, TIGIT, CD160, CD28, CD80, CD86, CTLA4, LAG3, LFA-1, LTβR, and HVEM.

11. The polypeptide of claim 10, wherein the second antigen is LTβR;
wherein (a) the domain capable of binding to the second antigen comprises a LTα or variant thereof;
(b) the domain capable of binding to the second antigen comprises a LTβ or variant thereof; or
(c) the domain capable of binding to the second antigen comprises a LTα or variant thereof and a LTβ or variant thereof.

12.-16. (canceled)

17. The polypeptide of claim 11, wherein
(a) the LTα or variant thereof comprises an amino acid sequence of SEO ID NO: 7, SEO ID NO: 8, or SEO ID NO: 9;
(b) the LTβ or variant thereof comprises an amino acid sequence of SEO ID NO: 10, SEO ID NO: 11, or SEO ID NO: 12; or
(c) the LTα or variant thereof comprises an amino acid sequence of SEQ ID NO: 7, SEQ ID NO: 8, or SEQ ID NO: 9; wherein the LTβ or variant thereof comprises an amino acid sequence of SEQ ID NO: 10, SEQ ID NO: 11, or SEQ ID NO: 12; or wherein the domain comprises an amino acid sequence of SEQ ID NO: 13, SEQ ID NO: 14, SEQ ID NO: 15, SEQ ID NO: 16, or SEQ ID NO: 20.

18. The polypeptide of claim 10, wherein the second antigen is HVEM; wherein the domain capable of binding to the second antigen comprises a LIGHT (TNFSF14) or variant thereof.

19. (canceled)

20. The polypeptide of claim 18, wherein the LIGHT or variant thereof comprises an amino acid sequence of SEQ ID NO: 17.

21. The polypeptide of claim 10, wherein the second antigen is CD40; wherein the domain capable of binding to the second antigen comprises an antibody or fragment thereof that binds CD40.

22. (canceled)

23. The polypeptide of claim 21, wherein the antibody or fragment thereof that binds CD40 comprises an amino acid sequence of SEQ ID NO: 21; or wherein the domain capable of binding to the second antigen comprises an amino acid sequence of SEO ID NO: 24.

24. (canceled)

25. The polypeptide of claim 10, wherein the second antigen is CLL1; wherein the domain capable of binding to the second antigen comprises an antibody or fragment thereof that binds CLL1.

26. (canceled)

27. The polypeptide of claim 25, wherein the antibody or fragment thereof that binds CLL1 comprises an amino acid sequence of SEQ ID NO: 22 or SEQ ID NO: 23.

28.-33. (canceled)

34. A polynucleotide comprising:
(a) a nucleic acid sequence encoding the polypeptide of claim 1; or
(b) a first region encoding a CAR comprising (i) an extracellular domain capable of binding to a first antigen, (ii) a transmembrane domain, and (iii) an intracellular domain; and a second region encoding a domain capable of binding to a second antigen expressed on the surface of a cell that can interact with a T cell, wherein the cell that can interact with the T cell is capable of presenting the first antigen to the T cell and/or inducing a response from the T cell upon interaction.

35. A vector comprising the polynucleotide of claim 34.

36. (canceled)

37. (canceled)

38. A CAR-T cell (1) comprising the polypeptide of claim 1; or (2) expressing:
(a) a CAR comprising (i) an extracellular domain capable of binding to a first antigen, (ii) a transmembrane domain, and (iii) an intracellular domain; and
(b) a domain capable of binding to a second antigen expressed on the surface of a cell that can interact with a T cell,
wherein the cell that can interact with the T cell is capable of presenting the first antigen to the T cell and/or inducing a response from the T cell upon interaction.

39.-64. (canceled)

65. A pharmaceutical composition, comprising the CAR-T cell of claim 38, and a pharmaceutically acceptable excipient.

66. A method for treating a disease or disorder in a subject comprising administering to the subject a therapeutically effective amount of the pharmaceutical composition of claim 65.

67.-70. (canceled)
